(19) United States
(12) Patent Application Publication
(10) Pub. No.: US 2024/0245711 A1
(43) Pub. Date: Jul. 25, 2024

(54) A 19-NOR C3,3-DISUBSTITUTED C21-N-PYRAZOLYL STEROID FOR THE TREATMENT OF MAJOR DEPRESSIVE DISORDER

(71) Applicant: Sage Therapeutics, Inc., Cambridge, MA (US)

(72) Inventors: Robert Alfonso Lasser, Brookline, MA (US); James Doherty, Bedford, MA (US); Jeffrey Martin Jonas, Vero Beach, FL (US); Stephen Jay Kanes, Swathmore, PA (US); Handan Gunduz-Bruce, Lexington, MA (US); Joi Lisa Dunbar, Needham, MA (US); Bambang Senoaji Adiwijaya, Lexington, MA (US)

(21) Appl. No.: 18/550,724

(22) PCT Filed: Mar. 17, 2022
(86) PCT No.: PCT/US2022/020716

§ 371 Date: Sep. 15, 2023

Related U.S. Application Data
(60) Provisional application No. 63/162,501, filed on Mar. 17, 2021.
(60) Provisional application No. 63/284,592, filed on Nov. 30, 2021.

(51) Int. Cl. A61K 31/58; A61P 25/24

(52) U.S. Cl. CPC A61K 31/58; A61P 25/24

(57) ABSTRACT

The present disclosure relates to methods of treating major depressive disorder (MDD) in a subject in need thereof by (i) performing an initial treatment course on the subject comprising administering a therapeutically effective amount of Compound (1), or a pharmaceutically acceptable salt thereof, and (ii) performing 0, 1, or 2 subsequent treatment courses on the subject, wherein each subsequent treatment course comprises administering a therapeutically effective amount of Compound (1), or a pharmaceutically acceptable salt thereof, in response to a recurrence of depression symptoms. The 0, 1, or 2 subsequent treatment courses can be performed over a period of 12 months from the beginning of the initial treatment course.

### CROSS-REFERENCE TO RELATED APPLICATIONS

This application claims the benefit of U.S. Provisional Application No. 63/162,501, filed on Mar. 17, 2021, and U.S. Provisional Application No. 63/284,592, filed on Nov. 30, 2021. The entire contents of the aforementioned applications are incorporated herein by reference in their entireties.

### FIELD OF THE INVENTION

The present disclosure is directed to a method of treating major depressive disorder (MDD) in a subject in need thereof, comprising: (i) performing an initial treatment course on the subject comprising administering a therapeutically effective amount of Compound (1), or a pharmaceutically acceptable salt thereof, and (ii) performing 0, 1, or 2 subsequent treatment courses on the subject, wherein each subsequent treatment course comprises administering a therapeutically effective amount of Compound (1), or a pharmaceutically acceptable salt thereof, in response to a recurrence of depression symptoms, wherein the 0, 1, or 2 subsequent treatment courses are performed over a period of 12 months from the beginning of the initial treatment course.

### BACKGROUND

GABA, γ-aminobutyric acid, has a profound influence on overall brain excitability because up to 40% of the neurons in the brain utilize GABA as a neurotransmitter. GABA interacts with its recognition site on the GRC (GABA receptor complex) to facilitate the flow of chloride ions down an electrochemical gradient of the GRC into the cell. An intracellular increase in the levels of this anion causes hyperpolarization of the transmembrane potential, rendering the neuron less susceptible to excitatory inputs (i.e., reduced neuron excitability). In other words, the higher the chloride ion concentration in the neuron, the lower the brain excitability (the level of arousal). It is well-documented that the GRC is responsible for the mediation of anxiety, seizure activity, and sedation. Thus, GABA and drugs that act like GABA (e.g., the therapeutically useful barbiturates and benzodiazepines (BZs), such as Valium®) produce their therapeutically useful effects by interacting with specific regulatory sites on the GRC.

Accumulated evidence has indicated that the GRC contains a distinct site for neuroactive steroids (Lan, N. C. et al., Neuwchem. Res. 16:347-356 (1991)). Neuroactive steroids can occur endogenously. The most potent endogenous neuroactive steroids are 3α-hydroxy-5-reduced pregnan-20-one and 3α-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232: 1004-1007 (1986); Harrison, N. L. et al., J. Pharmacol. Exp. Ther. 241:346-353 (1987)).

### SUMMARY OF THE INVENTION

In one aspect, the disclosure includes a method of treating major depressive disorder (MDD) in a subject in need thereof, comprising:
(i) performing an initial treatment course on the subject comprising administering a therapeutically effective amount of Compound (1):

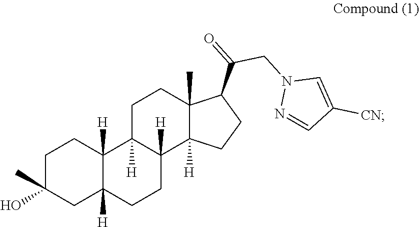

and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course.

In one aspect, the disclosure includes a method of treating major depressive disorder (MDD) in a subject in need thereof, comprising:
(i) performing an initial treatment course on the subject comprising administering a therapeutically effective amount of a pharmaceutically acceptable salt of Compound (1):

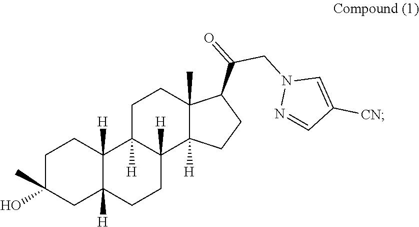

and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course.

In some embodiments of these aspects, 0 or 1 subsequent treatment courses are performed. In some embodiments, 1 subsequent treatment course is performed.

In some embodiments, there is at least about a 4 week, at least about a 6 week, or at least about an 8 week interval between the end of the initial treatment course and the beginning of the subsequent treatment course. In some embodiments, there is about a 4 week, about a 6 week, or about an 8 week interval between the end of the initial treatment course and the beginning of the subsequent treatment course.

In some embodiments, the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10 or a HAM-D score greater than or equal to 20.

In some embodiments, the initial treatment course has a duration of about 2 weeks or about 14 days. In some embodiments, each subsequent treatment course has a duration of about 2 weeks or about 14 days. In some embodiments, Compound (1), or the pharmaceutically acceptable salt of Compound (1), is administered once a day for about 14 days in the initial treatment course. In some embodiments, Compound (1), or the pharmaceutically acceptable salt of Compound (1), is administered once a day for about 14 days in each subsequent treatment course.

In some embodiments, Compound (1) is administered at a dose of about 20 mg to about 55 mg. In some embodiments, Compound (1) is administered at a dose of about 30 mg to about 50 mg. In some embodiments. Compound (1) is administered at a dose of about 50 mg. In some embodiments, wherein Compound (1) is administered at a dose of about 40 mg. In some embodiments, the pharmaceutically acceptable salt of Compound (1) is administered at a dose equivalent to about 20 mg to about 55 mg of the free base compound. In some embodiments, the pharmaceutically acceptable salt of Compound (1) is administered at a dose equivalent to about 30 mg to about 50 mg of the free base compound. In some embodiments, the pharmaceutically acceptable salt of Compound (1) is administered at a dose equivalent to about 50 mg of the free base compound. In some embodiments, the pharmaceutically acceptable salt of Compound (1) is administered at a dose equivalent to about 40 mg of the free base compound.

In some embodiments. Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments. Compound (1), or the pharmaceutically acceptable salt of Compound (1), is administered orally. In some embodiments, Compound (1), or the pharmaceutically acceptable salt of Compound (1), is administered with food. In some embodiments, the pharmaceutically acceptable salt of Compound (1), is administered once a day at night.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ. In some embodiments. Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the subject is treatment naïve.

In some embodiments, the subject has been on a stable dose of an additional antidepressant for at least 60 days prior to the beginning of the initial treatment course.

In another aspect, the disclosure includes a method of treating major depressive disorder (MDD) in a subject in need thereof, comprising:
(i) performing an initial treatment course on the subject comprising administering a therapeutically effective amount of Compound (1):

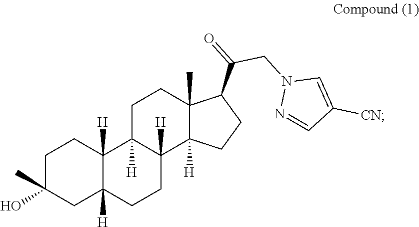

and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course, and
wherein the subject is treatment naïve.

In another aspect, the disclosure includes a method of treating major depressive disorder (MDD) in a subject in need thereof, comprising:
(i) performing an initial treatment course on the subject comprising administering a therapeutically effective amount of a pharmaceutically acceptable salt of Compound (1):

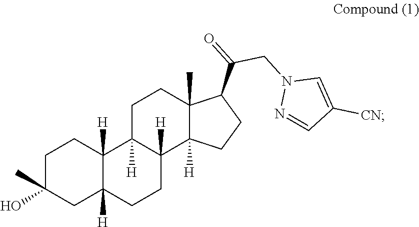

and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course, and
wherein the subject is treatment naïve.

In some embodiments of these aspects, 0 or 1 subsequent treatment courses are performed. In some embodiments, 1 subsequent treatment course is performed.

In some embodiments, there is at least about a 4 week, at least about a 6 week, or at least about an 8 week interval between the end of the initial treatment course and the beginning of the subsequent treatment course. In some embodiments, there is about a 4 week, about a 6 week, or about an 8 week interval between the end of the initial treatment course and the beginning of the subsequent treatment course.

In some embodiments, the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10 or a HAM-D score greater than or equal to 20.

In some embodiments, the initial treatment course has a duration of about 2 weeks or about 14 days. In some embodiments, each subsequent treatment course has a duration of about 2 weeks or about 14 days. In some embodiments, Compound (1), or the pharmaceutically acceptable salt of Compound (1), is administered once a day for about 14 days in the initial treatment course. In some embodiments, Compound (1), or the pharmaceutically acceptable salt of Compound (1), is administered once a day for about 14 days in each subsequent treatment course. In some embodiments, Compound (1) is administered at a dose of about 20 mg to about 55 mg. In some embodiments, Compound (1) is administered at a dose of about 30 mg to about 50 mg. In some embodiments. Compound (1) is administered at a dose of about 50 mg. In some embodiments, Compound (1) is administered at a dose of about 40 mg. In some embodiments, the pharmaceutically acceptable salt of Compound (1) is administered at a dose equivalent to about 20 mg to about 55 mg of the free base compound. In some embodiments, the pharmaceutically acceptable salt of Compound (1) is administered at a dose equivalent to about 30 mg to about 50 mg of the free base compound. In some embodiments, the pharmaceutically acceptable salt of Compound (1) is administered at a dose equivalent to about 50 mg of the free base compound. In some embodiments, the pharmaceutically acceptable salt of Compound (1) is administered at a dose equivalent to about 40 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally. In some embodiments, Compound (1), or the pharmaceutically acceptable salt of Compound (1), is administered with food. In some embodiments, Compound (1), or the pharmaceutically acceptable salt of Compound (1), is administered once a day at night.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In another aspect, the disclosure includes a method of treating major depressive disorder (MDD) in a subject in need thereof, comprising:
(i) performing an initial treatment course on the subject comprising administering about 30 mg to about 50 mg of Compound (1):

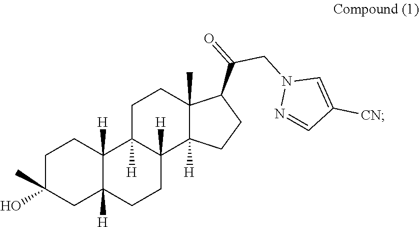

and
(ii) performing 0, 1, or 2 subsequent treatment courses on the subject, wherein each subsequent treatment course comprises administering about 30 mg to about 50 mg of Compound (1) to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound:

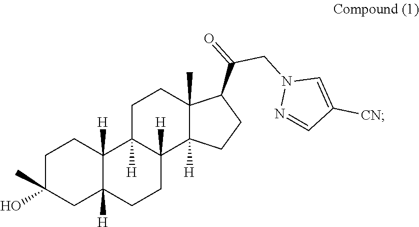

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

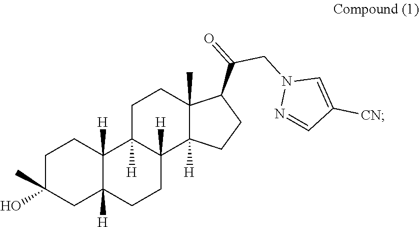

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

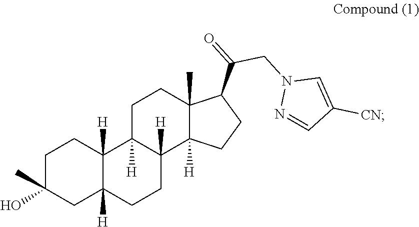

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

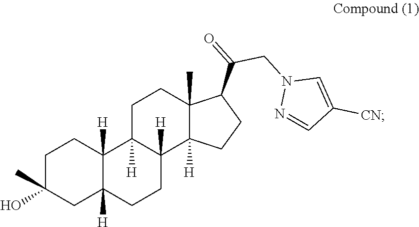

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

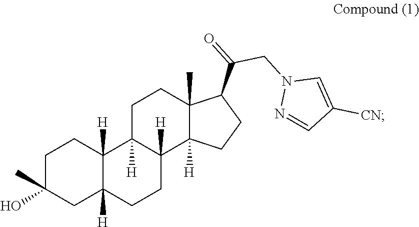

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

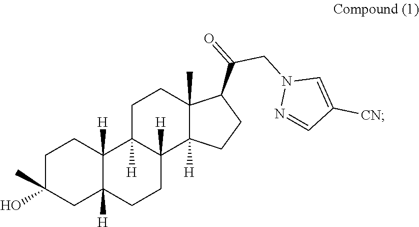

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

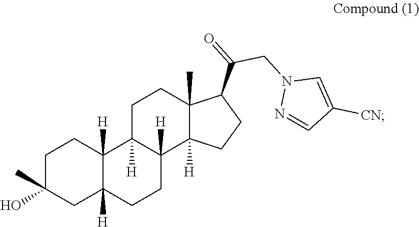

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

In another aspect, the disclosure includes a method of treating major depressive disorder (MDD) in a subject in need thereof, comprising:
(i) performing an initial treatment course on the subject comprising administering about 45 mg to about 55 mg of Compound (1):

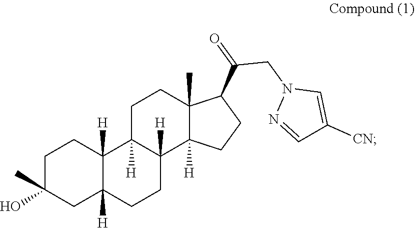

and
(ii) performing 0, 1, or 2 subsequent treatment courses on the subject, wherein each subsequent treatment course comprises administering about 45 mg to about 55 mg of Compound (1) to the subject in response to a recurrence of depression symptoms, wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound:

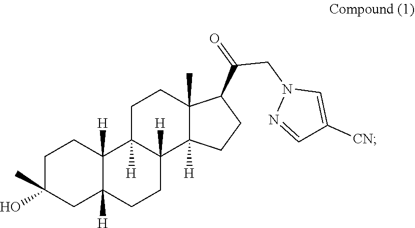

and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering about 45 mg to about 55 mg of Compound (1) once a day for about 14 days:

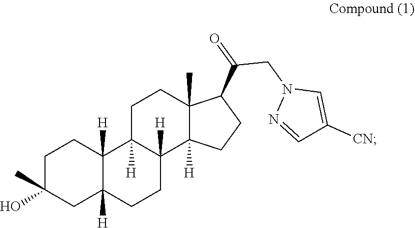

and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days:

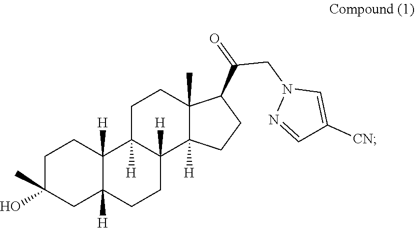

and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering about 45 mg to about 55 mg of Compound (1) once a day for about 14 days:

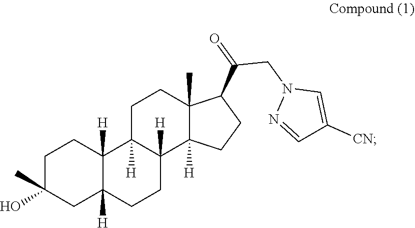

and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

In another aspect, the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days:

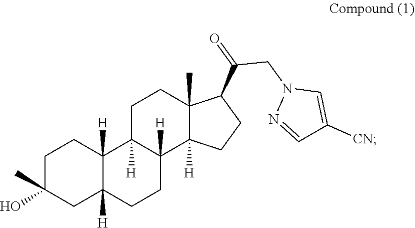

and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

In some embodiments of these aspects, 0 or 1 subsequent treatment courses are performed.

In some embodiments, the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10 or a HAM-D score greater than or equal to 20.

In some embodiments, Compound (1) is administered at a dose of about 50 mg or the pharmaceutically acceptable salt of Compound (1) is administered at a dose equivalent to about 50 mg of the free base compound. In some embodiments, Compound (1) is administered at a dose of about 40 mg or the pharmaceutically acceptable salt of Compound (1) is administered at a dose equivalent to about 40 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally. In some embodiments, Compound (1), or the pharmaceutically acceptable salt of Compound (1), is administered with food. In some embodiments, Compound (1), or the pharmaceutically acceptable salt of Compound (1), is administered once a day at night.

In some embodiments, the subject is treatment naïve.

### BRIEF DESCRIPTION OF THE DRAWINGS

FIG. 1 is an overview of the clinical study of Example 1.

FIG. 2 is a depiction of the eligibility criteria for clinical study of Example 1.

FIG. 3 is a flow chart of the dosing cohorts.

FIG. 4A is an overview of the flow of subjects (30 mg Cohort).

FIG. 4B is an overview of the flow of subjects (50 mg Cohort).

FIG. 5 shows HAM-D total score LS mean change from baseline over time for 30 mg and 50 mg Cohorts.

FIG. 6A is a bar graph of the HAMD-17 response at specific time points for 30 mg and 50 mg Cohorts.

FIG. 6B is a bar graph of the HAMD-17 remission at specific time points for 30 mg and 50 mg Cohorts.

FIG. 7 is a bar graph of the change in HAM-D total score over time—Study Period 1 (safety set).

FIG. 8 is a box plot of change from period-specific baseline in HAM-D total score at Day 15 for in each treatment cycle, by antidepressant use (yes/no) at period-specific baseline Low Dose Cohort (safety set for cycle 1, full analysis set for C2-5).

FIG. 9 shows box plots of change from baseline HAM-D total score at Day 15 in each treatment cycle (safety set for Treatment Cycle 1, FAS for C2-C5).

FIG. 10 is a box plot of change from period-specific baseline in HAM-D total score at Day 15 for in each treatment cycle, by antidepressant use (yes/no) at period-specific baseline Low Dose Cohort (safety set for Treatment Cycle 1, FAS for C2-C5).

FIG. 11 is a bar chart of HAM-D response over time—Study Period 1 (safety set).

FIG. 12 shows a bar chart of HAM-D response over time in Study Period 1, by antidepressant use at baseline (safety set).

FIG. 13 is a bar chart of HAM-D response at Day 15 in each treatment cycle, by antidepressant use at period-specific baseline—Low Dose Cohort (safety set).

FIG. 14 is a bar chart of HAM-D remission over time—Study Period 1 (safety set).

FIG. 15 shows a bar chart of HAM-D remission over time in Study Period 1, by antidepressant use at baseline (safety set).

FIG. 16 shows a bar chart of HAM-D remission at Day 15 in each treatment cycle, by antidepressant use at period-specific baseline—Low Dose Cohort.

FIG. 17 shows line plots of LS mean (±SE) change from baseline for subjects who had baseline HAM-D score ≥24—Study Period 1 (safety set).

FIG. 18 is a bar chart of HAM-D response over time Study Period 1 for subjects who had baseline HAM-D score ≥24 (safety set).

FIG. 19 is a bar chart of HAM-D remission for subjects who had baseline HAM-D score ≥24 over time—Study Period 1 (safety set).

FIG. 20 shows line plots of LS mean (±SE) change from baseline for subjects who had baseline HAM-D ≥26—Study Period 1 (safety set).

FIG. 21 is a bar chart of HAM-D response over time Study Period 1 for subjects who had baseline HAM-D score ≥26—Study Period 1 (safety set).

FIG. 22 is a bar chart of HAM-D remission over time for subjects who had baseline HAM-D score ≥26—Study Period 1 (safety set).

FIG. 23 provides box plots of time to each re-treatment by treatment cycles.

FIG. 24 is a bar chart of HAM-D response/non-responders with percent of responders in previous cycle—Low Dose Cohort (FAS).

FIG. 25 is a bar chart of HAM-D response/non-responders with percent of non-responders in previous cycle—Low Dose Cohort (FAS).

### DETAILED DESCRIPTION

### I. Definitions

As used herein, “Compound (1)” refers to the compound having the formula (or structure):

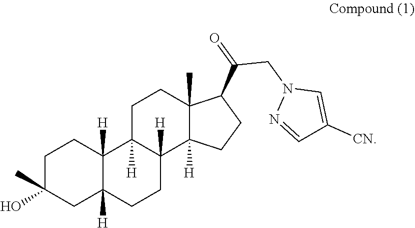

Compound (1) is also known as zuranolone, 3α-hydroxy-3β-methyl-21-(4-cyanopyrazol-1-yl)-5β-19-norpregnan-20-one, and by its IUPAC name: 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile (CAS Registry Number 1632051-40-1). A method of chemically synthesizing Compound (1), was described in U.S. Pat. No. 9,512,165 and PCT Application Publication No. WO 2014/169833; the entire contents of the aforementioned applications are incorporated herein by reference in their entireties. Several crystalline forms of Compound (1) and methods of preparing said forms were described in U.S. Pat. No. 11,236,121: U.S. Patent Application Publication No. US 2019/0177359; and PCT Application Publication No. WO 2018/039378; the entire contents of the aforementioned applications are incorporated herein by reference in their entireties. Pharmaceutical compositions of Compound (1) and methods of preparing said compositions were described in PCT Application Publication No. WO 2022/020363 and in U.S. application Ser. No. 17/579,541: the entire contents of the aforementioned applications are each incorporated herein by reference in its entirety.

Compound (1) is a neuroactive steroid that has been shown to be a positive allosteric modulator of GABA_{A} receptors that target synaptic and extrasynaptic GABA_{A} receptors. As a positive allosteric modulator of GABA_{A} receptors, Compound (1) serves as a therapeutic agent to treat CNS related disorders, e.g., depression, postpartum depression and major depressive disorder and to treat neurological conditions, e.g., essential tremor, epilepsy, and Parkinson's disease.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms refer to crystalline modifications comprising a given substance (e.g., Compound (1)), including single-component crystal forms and multiple-component crystal forms, and including, but not limited to, polymorphs, solvates, hydrates, and salts.

The term “substantially crystalline” refers to forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In some embodiments, the particular weight percent of crystallinity is at least 90%. In some embodiments, the particular weight percent of crystallinity is at least 95%. In some embodiments, Compound (1) can be a substantially crystalline sample of any of the crystalline forms described herein (e.g., crystalline Forms A and C) and/or PCT Application Publication No. WO 2018/039378; the entire contents of the aforementioned application are incorporated herein by reference in its entirety.

The term “substantially pure” relates to the composition of a specific crystalline form (e.g., a crystalline form of Compound (1)) that may be at least a particular weight percent free of impurities and/or other solid forms. Particular weight percentages may include 70%, 75%, 80%, 85%, 90%, 95%, 99%, or any percentage between 70% and 100%. In some embodiments, Compound (1) can be a substantially pure sample of any of the crystalline forms described herein, (e.g., crystalline Forms A and C). In some embodiments, Compound (1) can be substantially pure Form A. In some embodiments, Compound (1) can be substantially pure Form C.

As used herein, “XRPD” refers to X-ray powder diffraction. An XRPD pattern is an x-y graph with 2Q (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

The term “characteristic peaks” when referring to the peaks in an XRPD pattern of a crystalline form of a given chemical entity (e.g., a crystalline form of Compound (1)) refers to a collection of specific diffraction peaks whose values span a range of 2θ values (e.g., 0° to 40°) that are, as a whole, unique to that specific crystalline form.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid. 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid. 4-toluenesulfonic acid, camphorsulfonic acid, 4-methylbicyclo[2.2.2]-oct-2-ene-1-carboxylic acid, glucoheptonic acid. 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

The chemical elements are identified in accordance with the Periodic Table of the Elements, CAS version, Handbook of Chemistry and Physics, 75ili Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5ili Edition, John Wiley & Sons, Inc., New York, 2001: Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The terms “disease”, “disorder”, and “condition” are used interchangeably herein.

As used herein, the term “dose equivalent” means a bioequivalent dose. For example, the dose equivalent of a pharmaceutically acceptable salt of Compound (1) for a 50 mg dose of Compound (1) is the amount of the pharmaceutically acceptable salt (by weight) needed to provide a bioequivalent dose to the 50 mg dose of the free base of Compound (1).

As used herein, an “effective amount” of a compound (or pharmaceutically acceptable salt thereof) refers to an amount sufficient to elicit the desired biological response, e.g., to treat depression, e.g., major depressive disorder (MDD). As will be appreciated by those of ordinary skill in this art, the effective amount of a compound (or pharmaceutically acceptable salt thereof) of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, an “episodic dosing regimen” is a dosing regimen wherein a compound or a composition comprising a compound is administered to a subject for a finite period of time in response to the diagnosis of a disorder or symptom thereof, e.g., a diagnosis or symptom of depression or an episode of major depressive disorder. In some embodiments, the major depressive disorder is moderate major depressive disorder. In some embodiments, the major depressive disorder is severe major depressive disorder. In some embodiments, the compound is formulated as individual dosage units, each unit comprising Compound (1) and one or more suitable pharmaceutical excipient. In some embodiments, the episodic dosing regimen has a duration of a plurality of weeks, e.g. about 8 weeks. In contrast with chronic administration as defined herein, episodic dosing of a compound occurs over a finite period of time, e.g., from about 2 weeks to about 8 weeks, in response to a diagnosis or recurrence of a disorder, e.g., depression, or a symptom thereof. In some embodiments, episodic dosing occurs once per day across a plurality of weeks, e.g., from about 2 weeks to about 6 weeks. In one embodiment, the episodic dosing has a duration of two weeks. In some embodiments, more than one episodic dosing regimen, but no more than 3 episodic dosing regimens, is administered to the subject, e.g., two or more episodic regimens over a period of 12 months.

As used herein, the term “modulation” refers to the inhibition or potentiation of GABA_{A} receptor function. A “modulator” (e.g., a compound or pharmaceutically acceptable salt thereof that modulates GABA_{A} receptor function) may be, for example, an agonist, partial agonist, antagonist, or partial antagonist of the GABA_{A} receptor.

As used herein, “performing” an initial and/or subsequent treatment course is the act of carrying out the treatment course. In some embodiments, performing an initial and/or subsequent treatment course refers to beginning the administration of Compound (1), or a pharmaceutically acceptable salt thereof, to the subject. In some embodiments, performing an initial and/or subsequent treatment course, refers to completing the treatment course, e.g., administering Compound (1), or a pharmaceutically acceptable salt thereof, to the subject for a specific period of time (e.g., about 2 weeks or about 14 days).

As used herein, “safety set” refers to all subjects who were administered Compound (1) in the clinical study of Example 1. As used herein, “full analysis set” refers to all subjects in the safety set who have HAM-D response at Day 15 in treatment cycle 1 (e.g., initial treatment course) and discontinuation from study date, if it exists, is after the end of treatment cycle 1 (e.g., initial treatment course). A responder is a subject whose Day 15 HAM-D total score in the treatment cycle shows at least 50% reduction from baseline. If Day 15 HAM-D total score in the treatment cycle is missing, the subject is considered a non-responder.

As used herein, and unless otherwise specified, a “therapeutically effective amount” of a compound (or pharmaceutically acceptable salt thereof) is an amount sufficient to provide a therapeutic benefit in the treatment of a disease, disorder or condition, or to delay or minimize one or more symptoms associated with the disease, disorder or condition. A therapeutically effective amount of a compound (or pharmaceutically acceptable salt thereof) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, “solid dosage form” means a pharmaceutical dose(s) in solid form, e.g., tablets, capsules, granules, powders, sachets, reconstitutable powders, dry powder inhalers and chewables.

A “subject” or “patient” is a human (e.g., a male or female of any age group, e.g., a pediatric subject (e.g., infant, child, adolescent) or adult subject (e.g., young adult, middle-aged adult or senior adult)).

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

As used herein a “treatment course” refers to administration of Compound (1), or a pharmaceutically acceptable salt thereof, to treat major depressive disorder (MDD) in a subject in need thereof for a specific period of time. For example, a treatment course may administer Compound (1), or a pharmaceutically acceptable salt thereof, for about 2 weeks or about 14 days. The “treatment course” starts on the date the first dose of Compound (1), or a pharmaceutically acceptable salt thereof, is administered and goes up to the day upon which the last dose is administered. A subject receiving a treatment course for the first time within 12 months is receiving an “initial treatment course.”

A “treatment course” can be repeated, provided there is a time interval between each treatment course when Compound (1), or a pharmaceutically acceptable salt thereof, is not administered (e.g., at least 4 weeks, at least 6 weeks, or at least 8 weeks from the end of the initial treatment course). A “subsequent treatment course” refers to a treatment course performed on the subject after the subject has received an initial treatment course (e.g., repeating the treatment course). In some embodiments, 0), 1, or 2 subsequent treatment courses are performed over a period of 12 months from the beginning of the initial treatment course. Accordingly, in some embodiments, the methods provided herein may perform one, two, or three treatment courses on the subject over a period of 12 months from the beginning of the initial treatment course. In particular embodiments, a treatment course (e.g., a subsequent treatment course) may be repeated in response to a recurrence of depression symptoms. A recurrence of depression symptoms may be indicated by an evaluation of a subject using the Hamilton Rating Scale for Depression (HAM-D), Montgomery-Asberg Depression Rating Scale (MADRS), the Patient Health Questionnaire (PHQ-9), or a combination thereof. For example, a recurrence of depression symptoms in the subject may be indicated by a PHQ-9 score greater than or equal to 10 and/or by a HAM-D score greater than or equal to 20. In some embodiments, evaluations of the depression symptoms may be conducted every 14 days over a period of 12 months from the end of the initial treatment course.

As used herein, “treatment naïve” refers to a subject that has not been previously treated with the additional antidepressant within the current depressive episode. “Treatment naïve” also refers to a subject that has not taken any antidepressant within at least 60 days prior to the initial treatment course. In some embodiments, the subject is a “naïve patient.” The term “naïve patient” refers to two specific categories: i) patients with no previous therapeutic exposure to a type or category of medicament for treating depression (“primary naïve”), and ii) patients with previous exposure to a medicament for treating depression, but with a wash-out period of time adequately long based on the judgment of the practitioner (“secondary naïve”).

As used herein, the term “unit dosage form” is defined to refer to the form in which Compound (1) is administered to the subject. In some embodiments, the unit dosage form can be, for example, a pill, capsule, or tablet. In some embodiments, the unit dosage form is a capsule. In some embodiments, the typical amount of Compound (1) in a unit dosage form useful in the disclosure is about 10 mg to about 100 mg, about 20 mg to about 55 mg, or about 30 mg to about 50 mg (e.g., about, 10 mg, about 15 mg, about 20 mg, about 25 mg, about 30) mg, about 35 mg, about 40 mg, about 45 mg, about 50 mg, or about 55 mg).

In some embodiments, administering Compound (1) improves cognitive function. In some embodiments, the cognitive function refers to a collection of mental tasks and functions, including but not limited to: memory (e.g., semantic, episodic, procedural, priming, or working); orientation; language; problem solving; visual perception, construction, and integration: planning; organizational skills: selective attention: inhibitory control; and ability to mentally manipulate information. In one embodiment, the cognitive function is one or more selected from the group consisting of memory (e.g., semantic, episodic, procedural, priming, or working); orientation; language; problem solving; visual perception, construction, and integration: planning; organizational skills: selective attention: inhibitory control; and ability to mentally manipulate information. Measures of cognitive functioning include assessment tools designed to measure, for example: (a) general intelligence, (b) nonverbal intelligence, (c) achievement, (d) attention/executive functioning. (e) memory and learning. (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered).

### II. Methods of Treatment

The present disclosure is directed to methods of treating major depressive disorder (MDD). The diagnosis and severity of the major depressive disorder treated by the methods described herein can be characterized as defined by the Diagnostic and Statistical Manual of Mental Disorders, 5^{th} Edition (DSM-5).

### Depressive Disorders

Depressive disorders include disruptive mood dysregulation disorder, major depressive disorder (including major depressive episode), persistent depressive disorder (dysthymia), premenstrual dysphoric disorder, substance/medication-induced depressive disorder, depressive disorder due to another medical condition, other specified depressive disorder, and unspecified depressive disorder. The common feature of all of these disorders is the presence of sad, empty, or irritable mood, accompanied by somatic and cognitive changes that significantly affect the individual's capacity to function. What differs among them are issues of duration, timing, or presumed etiology.

Major depressive disorder represents the classic condition in this group of disorders. It is characterized by discrete episodes of at least 2 weeks' duration (although most episodes last considerably longer) involving clear-cut changes in affect, cognition, and neurovegetative functions and inter-episode remissions. A discrete episode of major depressive disorder may be referred to as a “major depressive episode” or “depressive episode”.

### Major Depressive Disorder (MDD)

Major depressive disorder is generally known in the art.

In some embodiments, MDD is also known as depression or clinical depression and it is a mood disorder that causes a persistent feeling of sadness and loss of interest. MDD affects how a subject may feel, think, and behave, and can lead to a variety of emotional and physical problems.

In some embodiments, MDD is defined and diagnosed according to the DSM-5, for example, MDD is diagnosed according to Criterion A, as described below.
Criterion A. Five (or more) of the following symptoms have been present during the same 2-week period and represent a change from previous functioning: at least one of the symptoms is either (1) depressed mood or (2) loss of interest or pleasure.
1. Depressed mood most of the day, nearly every day, as indicated by either subjective report (e.g., feels sad, empty, hopeless) or observation made by others (e.g., appears tearful). (Note: In children and adolescents, can be irritable mood.)
2. Markedly diminished interest or pleasure in all, or almost all, activities most of the day, nearly every day (as indicated by either subjective account or observation).
3. Significant weight loss when not dieting or weight gain (e.g., a change of more than 5% of body weight in a month), or decrease or increase in appetite nearly every day (Note: In children, consider failure to make expected weight gain.)
4. Insomnia or hypersomnia nearly every day.
5. Psychomotor agitation or retardation nearly every day (observable by others, not merely subjective feelings of restlessness or being slowed down).
6. Fatigue or loss of energy nearly every day.
7. Feelings of worthlessness or excessive or inappropriate guilt (which may be delusional) nearly every day (not merely self-reproach or guilt about being sick).
8. Diminished ability to think or concentrate, or indecisiveness, nearly every day (either by subjective account or as observed by others).
9. Recurrent thoughts of death (not just fear of dying), recurrent suicidal ideation without a specific plan, or a suicide attempt or a specific plan for committing suicide.
Criteria B-E, described below, are additional descriptions of MDD and may be considered for describing or diagnosing MDD, but are not required.
Criterion B. The symptoms cause clinically significant distress or impairment in social, occupational, or other important areas of functioning.
Criterion C. The episode is not attributable to the physiological effects of a substance or to another medical condition.
Criteria A-C can represent a major depressive episode.
Criterion D. The occurrence of the major depressive episode is not better explained by schizoaffective disorder, schizophrenia, schizophreniform disorder, delusional disorder, or other specified and unspecified schizophrenia spectrum and other psychotic disorders.
Criterion E. There has never been a manic episode or a hypomanic episode.

In some embodiments, a major depressive episode (MDE) is a period characterized by the symptoms of MDD as described above.

In some embodiments, MDD is a clinical course that is characterized by one or more major depressive episodes (MDE) in a subject.

In some embodiments, MDD is diagnosed according to Criteria A-C, as described above. In some embodiments, MDD is diagnosed according to Criteria A-E, as described above.

### Diagnostic Features

The criterion symptoms for major depressive disorder must be present nearly every day to be considered present, with the exception of weight change and suicidal ideation. Depressed mood must be present for most of the day, in addition to being present nearly every day. Often insomnia or fatigue is the presenting complaint, and failure to probe for accompanying depressive symptoms will result in underdiagnosis. Sadness may be denied at first but may be elicited through interview or inferred from facial expression and demeanor. With individuals who focus on a somatic complaint, clinicians should determine whether the distress from that complaint is associated with specific depressive symptoms. Fatigue and sleep disturbance are present in a high proportion of cases: psychomotor disturbances are much less common but are indicative of greater overall severity, as is the presence of delusional or near-delusional guilt.

The essential feature of a major depressive episode is a period of at least 2 weeks during which there is either depressed mood or the loss of interest or pleasure in nearly all activities (Criterion A above). In children and adolescents, the mood may be irritable rather than sad. The individual must also experience at least four additional symptoms drawn from a list that includes changes in appetite or weight, sleep, and psychomotor activity: decreased energy; feelings of worthlessness or guilt; difficulty thinking, concentrating, or making decisions; or recurrent thoughts of death or suicidal ideation or suicide plans or attempts. To count toward a major depressive episode, a symptom must either be newly present or must have clearly worsened compared with the person's pre-episode status. The symptoms must persist for most of the day, nearly every day, for at least 2 consecutive weeks. The episode must be accompanied by clinically significant distress or impairment in social, occupational, or other important areas of functioning. For some individuals with mild episodes, functioning may appear to be normal but requires markedly increased effort.

Sleep disturbance may take the form of either difficulty sleeping or sleeping excessively (Criterion A4). When insomnia is present, it typically takes the form of middle insomnia (i.e., waking up during the night and then having difficulty returning to sleep) or terminal insomnia (i.e., waking too early and being unable to return to sleep). Initial insomnia (i.e., difficulty falling asleep) may also occur. Individuals who present with over-sleeping (hypersomnia) may experience prolonged sleep episodes at night or increased daytime sleep. Sometimes the reason that the individual seeks treatment is for the disturbed sleep.

Accordingly, one aspect of the invention presents a method of treating major depressive disorder (MDD) in a subject in need thereof, comprising:
(i) performing an initial treatment course on the subject comprising administering a therapeutically effective amount of Compound (1):

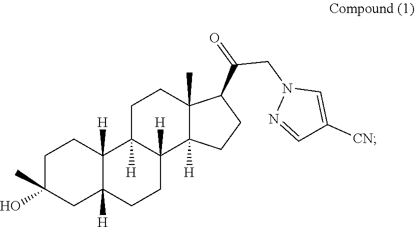

and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course.

Another aspect of the invention presents a method of treating major depressive disorder (MDD) in a subject in need thereof, comprising:
(i) performing an initial treatment course on the subject comprising administering a therapeutically effective amount of a pharmaceutically acceptable salt of Compound (1):

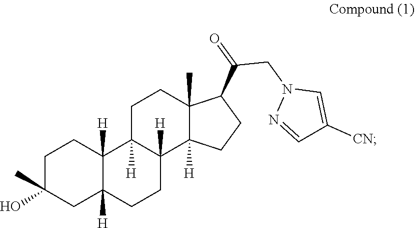

and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course.

In some embodiments, the major depressive disorder is moderate major depressive disorder. In some embodiments, the major depressive disorder is severe major depressive disorder.

In some embodiments, 0 or 1 subsequent treatment courses are performed. In some embodiments, no (e.g., 0) subsequent treatment course is performed. In some embodiments, 1 subsequent treatment course is performed. In some embodiments, 2 subsequent treatment course are performed.

In some embodiments, the method performs a total of one, two, or three treatment courses over a period of 12 months. In some embodiments, the method performs a total of two treatment courses over a period of 12 months from the beginning of the initial (first) treatment course. In some embodiments, the method performs a total of three treatment courses over a period of 12 months from the beginning of the initial (first) treatment course.

In some embodiments, there is at least about a 4 week, at least about a 6 week, or at least about an 8 week interval between the end of the initial treatment course and the beginning of the subsequent treatment course. In some embodiments, there is at least about a 4 week interval between the end of the initial treatment course and the beginning of the subsequent treatment course. In some embodiments, there is at least about a 6 week interval between the end of the initial treatment course and the beginning of the subsequent treatment course. In some embodiments, there is at least about an 8 week interval between the end of the initial treatment course and the beginning of the subsequent treatment course.

In some embodiments, there is about a 4 week, about a 6 week, or about an 8 week interval between the end of the initial treatment course and the beginning of the subsequent treatment course. In some embodiments, there is about a 4 week interval between the end of the initial treatment course and the beginning of the subsequent treatment course. In some embodiments, there is about a 6 week interval between the end of the initial treatment course and the beginning of the subsequent treatment course. In some embodiments, there is about an 8 week interval between the end of the initial treatment course and the beginning of the subsequent treatment course.

In embodiments where two subsequent treatment courses are performed, the time interval between each subsequent treatment course is the same as the aforementioned time interval between the initial treatment course and subsequent treatment course, e.g., there is at least about a 4 week, at least about a 6 week, or at least about an 8 week interval between the end of the first subsequent treatment course and the beginning of the second subsequent treatment course. In some embodiments, there is about a 4 week, about a 6 week, or about an 8 week interval between the end of the first subsequent treatment course and the beginning of the second subsequent treatment course.

In some embodiments, the recurrence of depression symptoms is indicated by an evaluation of the subject using the Hamilton Rating Scale for Depression (HAM-D). Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10. In some embodiments, the recurrence of depression symptoms in the subject is indicated by a HAM-D score greater than or equal to 20. In some embodiments, the recurrence of depression symptoms in the subject is indicated by a PHQ-9 score greater than or equal to 10 or a HAM-D score greater than or equal to 20.

In some embodiments, the initial treatment course has a duration of about 2 weeks or about 14 days. In some embodiments, the initial treatment course has a duration of about 2 weeks. In some embodiments, the initial treatment course has a duration of about 14 days. In some embodiments, the initial treatment course has a duration of 2 weeks or 14 days.

In some embodiments, each subsequent treatment course has a duration of about 2 weeks or about 14 days. In some embodiments, each subsequent treatment course has a duration of about 2 weeks. In some embodiments, each subsequent treatment course has a duration of about 14 days. In some embodiments, each subsequent treatment course has a duration of 2 weeks or 14 days.

In some embodiments. Compound (1), or the pharmaceutically acceptable salt of Compound (1), is administered once a day for about 2 weeks or about 14 days in the initial treatment course. In some embodiments, Compound (1), or the pharmaceutically acceptable salt of Compound (1), is administered once a day for about 2 weeks in the initial treatment course. In some embodiments, Compound (1), or the pharmaceutically acceptable salt of Compound (1), is administered once a day for about 14 days in the initial treatment course.

In some embodiments, Compound (1), or the pharmaceutically acceptable salt of Compound (1), is administered once a day for about 2 weeks or about 14 days in each subsequent treatment course. In some embodiments, Compound (1), or the pharmaceutically acceptable salt of Compound (1), is administered once a day for about 2 weeks in each subsequent treatment course. In some embodiments, Compound (1), or the pharmaceutically acceptable salt of Compound (1), is administered once a day for about 14 days in each subsequent treatment course.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments. Compound (1) is administered at a dose of about 20 mg to about 55 mg. In some embodiments, Compound (1) is administered at a dose of about 30 mg to about 50 mg. In some embodiments, Compound (1) is administered at a dose of about 45 mg to about 55 mg. In some embodiments. Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg.

In some embodiments, Compound (1) is administered at a dose of about 10 mg to about 100 mg once a day. In some embodiments, Compound (1) is administered at a dose of about 15 mg to about 75 mg once a day. In some embodiments, Compound (1) is administered at a dose of about 20 mg to about 60 mg once a day. In some embodiments. Compound (1) is administered at a dose of about 20 mg to about 55 mg once a day. In some embodiments, Compound (1) is administered at a dose of about 30 mg to about 50 mg once a day. In some embodiments, Compound (1) is administered at a dose of about 45 mg to about 55 mg once a day. In some embodiments, Compound (1) is administered at a dose of about 20 mg, about 25 mg, about 30 mg, about 35 mg, about 40 mg, about 45 mg, about 50 mg, about 55 mg, or about 60 mg once a day. In some embodiments, Compound (1) is administered at a dose of about 50) mg once a day. In some embodiments, Compound (1) is administered at a dose of about 40 mg once a day.

In some embodiments, Compound (1) is administered at a dose of about 20 mg to about 50 mg once a day for about 2 weeks or about 14 days. In some embodiments, Compound (1) is administered at a dose of about 30 mg to about 50 mg once a day for about 2 weeks or about 14 days. In some embodiments, Compound (1) is administered at a dose of about 45 mg to about 55 mg once a day for about 2 weeks or about 14 days. In some embodiments, Compound (1) is administered at a dose of about 50 mg once a day for less than 2 weeks. In some embodiments, Compound (1) is administered at a dose of about 50 mg once a day for about 2 weeks. In some embodiments, Compound (1) is administered at a dose of about 50 mg once a day for about 14 days. In some embodiments, Compound (1) is administered at a dose of about 40 mg once a day for less than 2 weeks. In some embodiments, Compound (1) is administered at a dose of about 40 mg once a day for about 2 weeks. In some embodiments. Compound (1) is administered at a dose of about 40 mg once a day for about 14 days.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 45 mg to about 55 mg of the free base compound. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound.

In some embodiments, the pharmaceutically acceptable salt of Compound (1) is administered at a dose equivalent of about 10 mg to about 100 mg of the free base compound once a day. In some embodiments, the pharmaceutically acceptable salt of Compound (1) is administered at a dose equivalent of about 15 mg to about 75 mg of the free base compound once a day. In some embodiments, the pharmaceutically acceptable salt of Compound (1) is administered at a dose equivalent of about 20 mg to about 60 mg of the free base compound once a day. In some embodiments, the pharmaceutically acceptable salt of Compound (1) is administered at a dose equivalent of about 20 mg to about 55 mg of the free base compound once a day. In some embodiments, the pharmaceutically acceptable salt of Compound (1) is administered at a dose equivalent of about 30 mg to about 50 mg of the free base compound once a day. In some embodiments, the pharmaceutically acceptable salt of Compound (1) is administered at a dose equivalent of about 45 mg to about 55 mg of the free base compound once a day. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) is administered at a dose equivalent of about 50 mg of the free base compound once a day. In some embodiments, the pharmaceutically acceptable salt of Compound (1) is administered at a dose equivalent of about 40 mg of the free base compound once a day.

In some embodiments, the pharmaceutically acceptable salt of Compound (1) is administered at a dose equivalent of about 30 mg to about 50 mg of the free base compound once a day for about 2 weeks or about 14 days. In some embodiments, the pharmaceutically acceptable salt of Compound (1) is administered at a dose equivalent of about 45 mg to about 55 mg of the free base compound once a day for about 2 weeks or about 14 days. In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 2 weeks. In some embodiments, the pharmaceutically acceptable salt of Compound (1) is administered at a dose equivalent of about 50 mg of the free base compound once a day for about 14 days. In some embodiments, the pharmaceutically acceptable salt of Compound (1) is administered at a dose equivalent of about 40 mg of the free base compound once a day for less than 2 weeks. In some embodiments, the pharmaceutically acceptable salt of Compound (1) is administered at a dose equivalent of about 40 mg of the free base compound once a day for about 2 weeks. In some embodiments, the pharmaceutically acceptable salt of Compound (1) is administered at a dose equivalent of about 40 mg of the free base compound once a day for about 14 days.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered chronically.

In some embodiments. Compound (1), or the pharmaceutically acceptable salt of Compound (1), is administered in one or more capsules. In some embodiments, the therapeutically effective amount is administered across two capsules. In some embodiments, the therapeutically effective amount is administered across three capsules.

In some embodiments. Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps. In some embodiments, the subject is administered Compound (1), or the pharmaceutically acceptable salt of Compound (1), once a day at night. In some embodiments, the subject is administered Compound (1), or the pharmaceutically acceptable salt of Compound (1), once a day no later than 1 hour before the patient sleeps. In some embodiments, the subject is administered Compound (1), or the pharmaceutically acceptable salt of Compound (1), once a day no later than 15 minutes before the patient sleeps.

In some embodiments, Compound (1) is in a crystalline form. In some embodiments, the crystalline form of Compound (1) is any crystalline form disclosed in PCT Application Publication No. WO 2018/039378: the entire contents of the aforementioned application are incorporated herein by reference in its entirety.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments. Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the crystalline form of Compound (1) comprises a mixture of two or more crystalline forms.

In some embodiments, the subject is treatment naïve. In some embodiments, the subject has not received any antidepressant treatment within at least 30 days prior to the start of the initial treatment course. In some embodiments, the subject has not received any antidepressant treatment within at least 60 days prior to the start of the initial treatment course.

In some embodiments, the subject has been on a stable dose of an additional antidepressant for at least 60 days prior to the beginning of the initial treatment course.

Another aspect of the disclosure includes a method of treating major depressive disorder (MDD) in a subject in need thereof, comprising:
(i) performing an initial treatment course on the subject comprising administering a therapeutically effective amount of Compound (1):

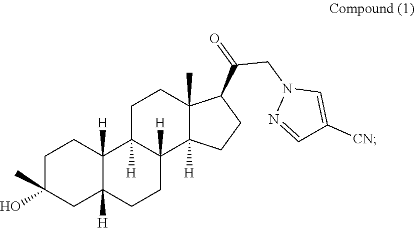

and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course, and
wherein the subject is treatment naïve.

Another aspect of the disclosure includes a method of treating major depressive disorder (MDD) in a subject in need thereof, comprising:
(i) performing an initial treatment course on the subject comprising administering a therapeutically effective amount of a pharmaceutically acceptable salt of Compound (1):

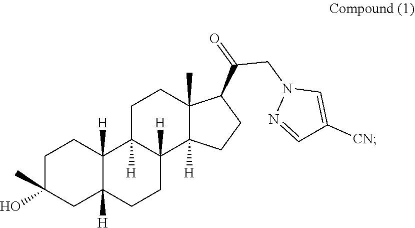

and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course, and
wherein the subject is treatment naïve.

In some embodiments, the major depressive disorder is moderate major depressive disorder. In some embodiments, the major depressive disorder is severe major depressive disorder.

In some embodiments, 0 or 1 subsequent treatment courses are performed. In some embodiments, no (e.g., 0) subsequent treatment course is performed. In some embodiments, 1 subsequent treatment course is performed. In some embodiments, 2 subsequent treatment courses are performed.

In some embodiments, the method performs a total of one, two, or three treatment courses over a period of 12 months. In some embodiments, the method performs a total of two treatment courses over a period of 12 months from the beginning of the initial (first) treatment course. In some embodiments, the method performs a total of three treatment courses over a period of 12 months from the beginning of the initial (first) treatment course.

In some embodiments, there is at least about a 4 week, at least about a 6 week, or at least about an 8 week interval between the end of the initial treatment course and the beginning of the subsequent treatment course. In some embodiments, there is at least about a 4 week interval between the end of the initial treatment course and the beginning of the subsequent treatment course. In some embodiments, there is at least about a 6 week interval between the end of the initial treatment course and the beginning of the subsequent treatment course. In some embodiments, there is at least about an 8 week interval between the end of the initial treatment course and the beginning of the subsequent treatment course.

In some embodiments, there is about a 4 week, about a 6 week, or about an 8 week interval between the end of the initial treatment course and the beginning of the subsequent treatment course. In some embodiments, there is about a 4 week interval between the end of the initial treatment course and the beginning of the subsequent treatment course. In some embodiments, there is about a 6 week interval between the end of the initial treatment course and the beginning of the subsequent treatment course. In some embodiments, there is about an 8 week interval between the end of the initial treatment course and the beginning of the subsequent treatment course.

In embodiments where two subsequent treatment courses are performed, the time interval between each subsequent treatment course is the same as the aforementioned time interval between the initial treatment course and subsequent treatment course, e.g., there is at least about a 4 week, at least about a 6 week, or at least about an 8 week interval between the end of the first subsequent treatment course and the beginning of the second subsequent treatment course. In some embodiments, there is about a 4 week, about a 6 week, or about an 8 week interval between the end of the first subsequent treatment course and the beginning of the second subsequent treatment course.

In some embodiments, the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10. In some embodiments, the recurrence of depression symptoms in the subject is indicated by a HAM-D score greater than or equal to 20. In some embodiments, the recurrence of depression symptoms in the subject is indicated by a PHQ-9 score greater than or equal to 10 or a HAM-D score greater than or equal to 20.

In some embodiments, the initial treatment course has a duration of about 2 weeks or about 14 days. In some embodiments, the initial treatment course has a duration of about 2 weeks. In some embodiments, the initial treatment course has a duration of about 14 days. In some embodiments, the initial treatment course has a duration of 2 weeks or 14 days.

In some embodiments, each subsequent treatment course has a duration of about 2 weeks or about 14 days. In some embodiments, each subsequent treatment course has a duration of about 2 weeks. In some embodiments, each subsequent treatment course has a duration of about 14 days. In some embodiments, each subsequent treatment course has a duration of 2 weeks or 14 days.

In some embodiments, Compound (1), or the pharmaceutically acceptable salt of Compound (1), is administered once a day for about 2 weeks or about 14 days in the initial treatment course. In some embodiments, Compound (1), or the pharmaceutically acceptable salt of Compound (1), is administered once a day for about 2 weeks in the initial treatment course. In some embodiments, Compound (1), or the pharmaceutically acceptable salt of Compound (1), is administered once a day for about 14 days in the initial treatment course.

In some embodiments, Compound (1), or the pharmaceutically acceptable salt of Compound (1), is administered once a day for about 2 weeks or about 14 days in each subsequent treatment course. In some embodiments, Compound (1), or the pharmaceutically acceptable salt of Compound (1), is administered once a day for about 2 weeks in each subsequent treatment course. In some embodiments, Compound (1), or the pharmaceutically acceptable salt of Compound (1), is administered once a day for about 14 days in each subsequent treatment course.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg to about 55 mg. In some embodiments, Compound (1) is administered at a dose of about 30 mg to about 50 mg. In some embodiments, Compound (1) is administered at a dose of about 45 mg to about 55 mg. In some embodiments. Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg.

In some embodiments, Compound (1) is administered at a dose of about 10 mg to about 100 mg once a day. In some embodiments. Compound (1) is administered at a dose of about 15 mg to about 75 mg once a day. In some embodiments, Compound (1) is administered at a dose of about 20 mg to about 60 mg once a day. In some embodiments, Compound (1) is administered at a dose of about 20 mg to about 55 mg once a day. In some embodiments, Compound (1) is administered at a dose of about 30 mg to about 50 mg once a day. In some embodiments, Compound (1) is administered at a dose of about 45 mg to about 55 mg once a day. In some embodiments, Compound (1) is administered at a dose of about 20 mg, about 25 mg, about 30 mg, about 35 mg, about 40 mg, about 45 mg, about 50 mg, about 55 mg, or about 60 mg once a day. In some embodiments, Compound (1) is administered at a dose of about 50 mg once a day. In some embodiments. Compound (1) is administered at a dose of about 40 mg once a day.

In some embodiments, Compound (1) is administered at a dose of about 20 mg to about 50 mg once a day for about 2 weeks or about 14 days. In some embodiments, Compound (1) is administered at a dose of about 30 mg to about 50 mg once a day for about 2 weeks or about 14 days. In some embodiments, Compound (1) is administered at a dose of about 45 mg to about 55 mg once a day for about 2 weeks or about 14 days. In some embodiments, Compound (1) is administered at a dose of about 50 mg once a day for less than 2 weeks. In some embodiments, Compound (1) is administered at a dose of about 50 mg once a day for about 2 weeks. In some embodiments, Compound (1) is administered at a dose of about 50 mg once a day for about 14 days. In some embodiments, Compound (1) is administered at a dose of about 40 mg once a day for less than 2 weeks. In some embodiments, Compound (1) is administered at a dose of about 40 mg once a day for about 2 weeks. In some embodiments, Compound (1) is administered at a dose of about 40 mg once a day for about 14 days.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 45 mg to about 55 mg of the free base compound. In some embodiments, the pharmaceutically acceptable salt of Compound (1) is administered at a dose equivalent of about 20 mg, about 25 mg, about 30) mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound.

In some embodiments, the pharmaceutically acceptable salt of Compound (1) is administered at a dose equivalent of about 10 mg to about 100 mg of the free base compound once a day. In some embodiments, the pharmaceutically acceptable salt of Compound (1) is administered at a dose equivalent of about 15 mg to about 75 mg of the free base compound once a day. In some embodiments, the pharmaceutically acceptable salt of Compound (1) is administered at a dose equivalent of about 20 mg to about 60 mg of the free base compound once a day. In some embodiments, the pharmaceutically acceptable salt of Compound (1) is administered at a dose equivalent of about 20 mg to about 55 mg of the free base compound once a day. In some embodiments, the pharmaceutically acceptable salt of Compound (1) is administered at a dose equivalent of about 30 mg to about 50 mg of the free base compound once a day. In some embodiments, the pharmaceutically acceptable salt of Compound (1) is administered at a dose equivalent of about 45 mg to about 55 mg of the free base compound once a day. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) is administered at a dose equivalent of about 50 mg of the free base compound once a day. In some embodiments, the pharmaceutically acceptable salt of Compound (1) is administered at a dose equivalent of about 40 mg of the free base compound once a day.

In some embodiments, the pharmaceutically acceptable salt of Compound (1) is administered at a dose equivalent of about 30 mg to about 50 mg of the free base compound once a day for about 2 weeks or about 14 days. In some embodiments, the pharmaceutically acceptable salt of Compound (1) is administered at a dose equivalent of about 45 mg to about 55 mg of the free base compound once a day for about 2 weeks or about 14 days. In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 2 weeks. In some embodiments, the pharmaceutically acceptable salt of Compound (1) is administered at a dose equivalent of about 50 mg of the free base compound once a day for about 14 days. In some embodiments, the pharmaceutically acceptable salt of Compound (1) is administered at a dose equivalent of about 40 mg of the free base compound once a day for less than 2 weeks. In some embodiments, the pharmaceutically acceptable salt of Compound (1) is administered at a dose equivalent of about 40 mg of the free base compound once a day for about 2 weeks. In some embodiments, the pharmaceutically acceptable salt of Compound (1) is administered at a dose equivalent of about 40 mg of the free base compound once a day for about 14 days.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments, Compound (1), or the pharmaceutically acceptable salt of Compound (1), is administered chronically.

In some embodiments, Compound (1), or the pharmaceutically acceptable salt of Compound (1), is administered in one or more capsules. In some embodiments, the therapeutically effective amount is administered across two capsules. In some embodiments, the therapeutically effective amount is administered across three capsules.

In some embodiments, Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps. In some embodiments, the subject is administered Compound (1), or the pharmaceutically acceptable salt of Compound (1), once a day at night. In some embodiments, the subject is administered Compound (1), or the pharmaceutically acceptable salt of Compound (1), once a day no later than 1 hour before the patient sleeps. In some embodiments, the subject is administered Compound (1), or the pharmaceutically acceptable salt of Compound (1), once a day no later than 15 minutes before the patient sleeps.

In some embodiments, Compound (1) is in a crystalline form. In some embodiments, the crystalline form Compound (1) is any crystalline form disclosed in PCT Application Publication No. WO 2018/039378; the entire contents of the aforementioned application are incorporated herein by reference in its entirety.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the crystalline form of Compound (1) comprises a mixture of two or more crystalline forms.

Another aspect of the disclosure includes a method of treating major depressive disorder (MDD) in a subject in need thereof, comprising:
(i) performing an initial treatment course on the subject comprising administering about 30 mg to about 50 mg of Compound (1):

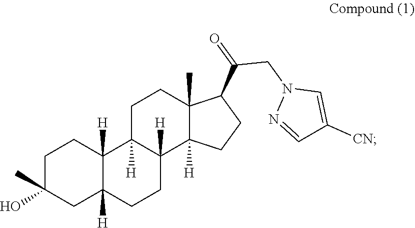

and
(ii) performing 0, 1, or 2 subsequent treatment courses on the subject, wherein each subsequent treatment course comprises administering about 30 mg to about 50 mg of Compound (1) to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound:

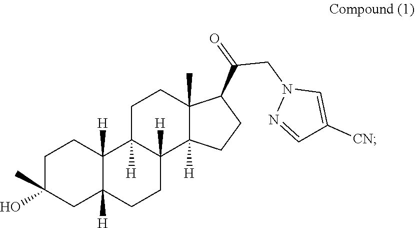

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

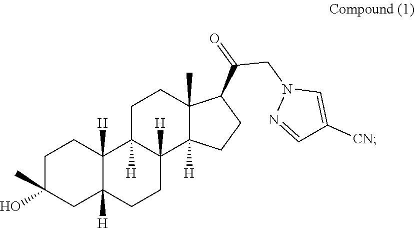

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

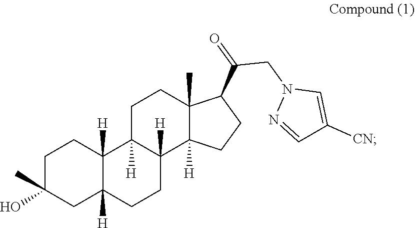

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

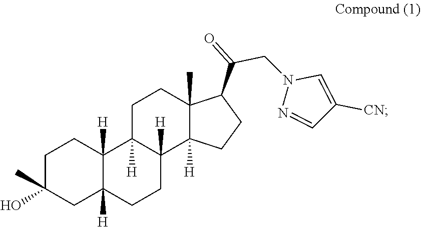

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

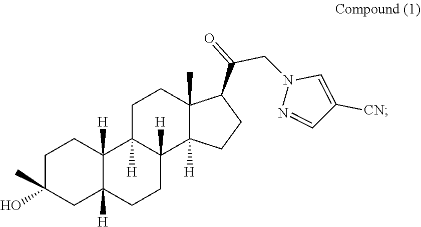

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering about 30 mg to about 50 mg of Compound (1) once a day for about 14 days:

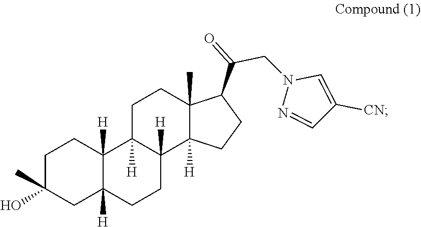

and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days:

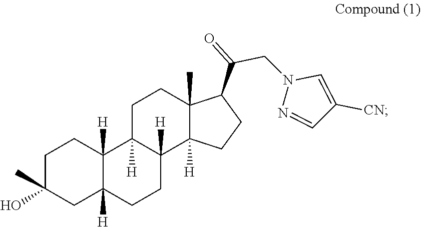

and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

Another aspect of the disclosure includes a method of treating major depressive disorder (MDD) in a subject in need thereof, comprising:
(i) performing an initial treatment course on the subject comprising administering about 45 mg to about 55 mg of Compound (1):

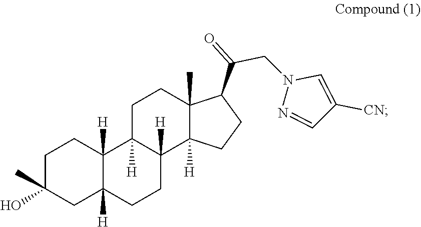

and
(ii) performing 0, 1, or 2 subsequent treatment courses on the subject, wherein each subsequent treatment course comprises administering about 45 mg to about 55 mg of Compound (1) to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound:

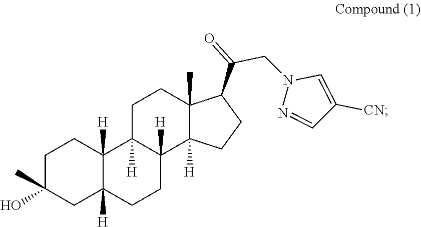

and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering about 45 mg to about 55 mg of Compound (1) once a day for about 14 days:

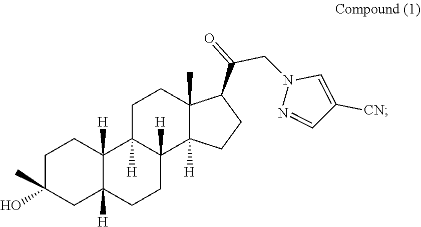

and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days:

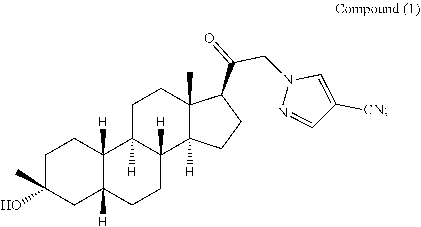

and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering about 45 mg to about 55 mg of Compound (1) once a day for about 14 days:

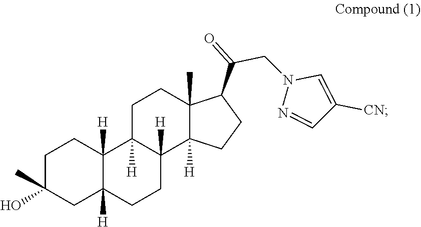

and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

Another aspect of the disclosure includes a method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days:

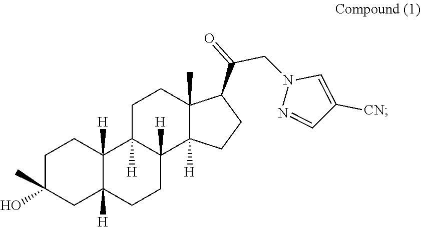

and
(ii) performing 0), 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms, provided there is at least about a 6 week interval between the end of the initial treatment course and the beginning of the subsequent treatment course,
wherein the 0), 1, or 2 subsequent treatment courses are performed over a period of 12 months from the beginning of the initial treatment course.

In some embodiments, the major depressive disorder is moderate major depressive disorder. In some embodiments, the major depressive disorder is severe major depressive disorder.

In some embodiments, 0) or 1 subsequent treatment courses are performed. In some embodiments, no (e.g., 0) subsequent treatment course is performed. In some embodiments, 1 subsequent treatment course is performed. In some embodiments, 2 subsequent treatment courses are performed.

In some embodiments, the method performs a total of one, two, or three treatment courses over a period of 12 months. In some embodiments, the method performs a total of two treatment courses over a period of 12 months from the beginning of the initial (first) treatment course. In some embodiments, the method performs a total of three treatment courses over a period of 12 months from the beginning of the initial (first) treatment course.

In some embodiments, the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof. In some embodiments, the recurrence of depression symptoms in the subject is indicated by a PHQ-9 score greater than or equal to 10. In some embodiments, the recurrence of depression symptoms in the subject is indicated by a HAM-D score greater than or equal to 20. In some embodiments, the recurrence of depression symptoms in the subject is indicated by a PHQ-9 score greater than or equal to 10 or a HAM-D score greater than or equal to 20.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg to about 55 mg. In some embodiments, Compound (1) is administered at a dose of about 30 mg to about 50 mg. In some embodiments. Compound (1) is administered at a dose of about 45 mg to about 55 mg. In some embodiments. Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg.

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg to about 55 mg of the free base compound. In some embodiments, the pharmaceutically acceptable salt of Compound (1) is administered at a dose equivalent of about 30 mg to about 50 mg of the free base compound. In some embodiments, the pharmaceutically acceptable salt of Compound (1) is administered at a dose equivalent of about 45 mg to about 55 mg of the free base compound. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound.

In some embodiments, Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1), or the pharmaceutically acceptable salt of Compound (1), is administered orally.

In some embodiments. Compound (1), or the pharmaceutically acceptable salt of Compound (1), is administered with food. In some embodiments, Compound (1), or the pharmaceutically acceptable salt of Compound (1), is administered with fat-containing food. Examples of fat-containing food include nuts, peanut butter, avocado, eggs, and cheese. In some embodiments, Compound (1), or the pharmaceutically acceptable salt of Compound (1), is administered at night with fat-containing food (e.g., within 1 hour of an evening meal which contains fat, or with a fat-containing snack).

In some embodiments, the subject is administered Compound (1), or the pharmaceutically acceptable salt of Compound (1), at night. In some embodiments, the subject is administered Compound (1), or the pharmaceutically acceptable salt of Compound (1), no later than 1 hour before the patient sleeps. In some embodiments, the subject is administered Compound (1), or the pharmaceutically acceptable salt of Compound (1), no later than 15 minutes before the patient sleeps.

In some embodiments, the subject is treatment naïve. In some embodiments, the subject has not received any antidepressant treatment within at least 30 days prior to the start of the initial treatment course. In some embodiments, the subject has not received any antidepressant treatment within at least 60 days prior to the start of the initial treatment course.

### III. Pharmaceutical Compositions

Another aspect of the disclosure provides a pharmaceutical composition comprising Compound (1) (also referred to as the “active ingredient”), and a pharmaceutically acceptable excipient for use in the methods described herein. In another aspect, the disclosure provides a pharmaceutical composition comprising a pharmaceutically acceptable salt of the active ingredient and a pharmaceutically acceptable excipient for use in the methods described herein. In certain embodiments, the pharmaceutical composition comprises an effective amount of the active ingredient or a pharmaceutically acceptable salt of the active ingredient. In certain embodiments, the pharmaceutical composition comprises a therapeutically effective amount of the active ingredient or a pharmaceutically acceptable salt of the active ingredient. In some embodiments, the pharmaceutical composition of Compound (1) is any pharmaceutical composition disclosed in PCT Application Publication No. WO 2022/020363: the entire contents of the aforementioned application are incorporated herein by reference in its entirety.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical composition is administered orally.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials, as well as processing techniques and the like, are set forth in Part 8 of Remington's Pharmaceutical Sciences, 17^{th} edition, 1985, Mack Publishing Company, Easton, Pennsylvania, which is incorporated herein by reference.

The compositions of the present invention can also be administered in sustained release forms or from sustained release drug delivery systems. A description of representative sustained release materials can be found in Remington's Pharmaceutical Sciences.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

In another aspect, the disclosure includes a method of treating major depressive disorder in a subject in need thereof, the method comprising administering to the subject a daily dose comprising 45 mg to 55 mg of Compound (1) using an episodic dosing regimen to treat major depressive disorder in the subject.

In one embodiment of this aspect, the episodic dosing regimen has a duration of about 2 to about 8 weeks. In another embodiment, the episodic dosing regimen has a duration of about 2 to about 6 weeks. In another embodiment, the episodic dosing regimen has a duration of about 2 to about 4 weeks. In a further embodiment, the episodic dosing regimen has a duration of about 2 weeks or 14 days. In still a further embodiment, the episodic dosing regimen has a duration of 2 weeks.

In one embodiment, the subject exhibits a response to the episodic dosing regimen, wherein the response is indicated by greater than or equal to about 50% reduction in HAM-D score from baseline. In one embodiment, the subject is evaluated for recurrence, or reappearance of depression symptoms.

In some embodiments, the method comprises a plurality of episodic dosing regimen. In one embodiment, the episodic dosing regimens are spaced apart by at least an 8 week interval. In another embodiment, the daily dose comprises 45 mg to 55 mg of Compound (1). In another embodiment, the daily dose comprises 48 mg to 52 mg of Compound (1). In a further embodiment, the daily dose comprises 50 mg of Compound (1).

In one embodiment, the daily dose is administered to the subject in the evening. In another embodiment, the daily dose is administered to the subject concurrently with, or immediately after ingestion of food.

In another aspect, the disclosure includes a method of treating major depressive disorder in a subject in need thereof, the method comprising the steps of:
(i) administering once daily to the subject a daily dose comprising 45 mg to 55 mg of Compound (1) for about two weeks; and
(ii) re-administering once daily to the subject a daily dose comprising 45 mg to 55 mg of Compound (1) for about two weeks in response to a recurrence of depression symptoms, provided there is at least an 8 week interval between administration of Compound (1) to the subject and re-administration of Compound (1) to the subject.

In one embodiment of this aspect, Compound (1) is re-administered to the subject for 2 weeks. In another embodiment, the interval between administration of Compound (1) to the subject and re-administration of Compound (1) to the subject is 8 weeks. In a further embodiment, the major depressive disorder is moderate major depressive disorder. In another further embodiment, the major depressive disorder is severe major depressive disorder. In one embodiment, the subject has been experiencing a major depressive episode over about a 1-year period. In another embodiment, the subject is between about 18 and about 75 years of age. In another embodiment, the subject is between about 18 and about 65 years of age. In one embodiment, the daily dose comprises 48 mg to 52 mg of Compound (1). In a further embodiment, the daily dose comprises 50 mg of Compound (1). In another embodiment, the daily dose is administered to the subject in the evening. In another embodiment, the daily dose is administered to the subject concurrently with, or immediately after ingestion of food. In one embodiment, the daily dose comprising Compound (1) is in the form of a capsule. In one embodiment, the method further comprises administering to the subject a second therapeutic agent.

In another aspect, the disclosure includes a method of treating major depressive disorder in a subject in need thereof, using a kit comprising:
a plurality of individual dosage units, each comprising 45 mg to 55 mg of Compound (1), and
an instruction set, wherein the instruction set describes a method for administering the dosage units to the subject using an episodic dosing regimen.

In another aspect, the disclosure includes a method of treating major depressive disorder in a subject in need thereof, using a kit comprising:
a plurality of individual dosage units, each comprising 30 mg to 50 mg of Compound (1), and
an instruction set, wherein the instruction set describes a method for administering the dosage units to the subject using an episodic dosing regimen.

In embodiments of these aspects, the episodic dosing regimen has a duration of about 2 to about 8 weeks. In one embodiment, the episodic dosing regimen has a duration of about 2 to about 6 weeks. In another embodiment, the episodic dosing regimen has a duration of about 2 to about 4 weeks. In a further embodiment, the episodic dosing regimen has a duration of about 2 weeks. In still a further embodiment, the episodic dosing regimen has a duration of 2 weeks. In one embodiment, the subject has been diagnosed with major depressive disorder. In a further embodiment, the major depressive disorder is moderate major depressive disorder. In another further embodiment, the major depressive disorder is severe major depressive disorder. In one embodiment, each dosage unit comprises 45 mg to 55 mg of Compound (1). In one embodiment, each dosage unit comprises 48 mg to 52 mg of Compound (1). In a further embodiment, each dosage unit comprises 50 mg of Compound (1). In a further embodiment, each dosage unit comprises 40 mg of Compound (1). In one embodiment, the method described by the instruction set includes instructions to administer the dosage unit in the evening. In another embodiment, the method described by the instruction set includes instructions to administer the dosage unit concurrently with, or immediately after ingestion of food.

In another aspect, the disclosure includes a kit comprising a plurality of dosages, each comprising 45 mg to 55 mg of Compound (1), and an instruction set describing a method of administering the dosages using an episodic dosing regimen for treating major depressive disorder. In one embodiment of this aspect, the dosages are individual dosage units of Compound (1). In a further embodiment, an individual dosage unit comprises 48 mg to 52 mg of Compound (1). In still a further embodiment, an individual dosage unit comprises 50 mg of Compound (1). In one embodiment, the episodic dosing regimen has a duration of about 2 to about 8 weeks. In another embodiment, the episodic dosing regimen has a duration of about 2 to about 6 weeks. In a further embodiment, the episodic dosing regimen has a duration of about 2 to about 4 weeks. In still a further embodiment, the episodic dosing regimen has a duration of about 2 weeks or 14 days. In yet a further embodiment, the episodic dosing regimen has a duration of 2 weeks. In one embodiment, the major depressive disorder is moderate major depressive disorder. In another embodiment, the major depressive disorder is severe major depressive disorder. In one embodiment, the instruction set is printed on a suitable material. In another embodiment, the individual dosage units are capsules or tablets. In a further embodiment, the individual dosage unit is a capsule. In some embodiments, the individual dosage unit is a capsule of size 1, 2, 3, or 4. In one embodiment, the capsule is size 1.

In another aspect, the disclosure includes a kit comprising a plurality of dosages, each comprising 30 mg to 50 mg of Compound (1), and an instruction set describing a method of administering the dosages using an episodic dosing regimen for treating major depressive disorder. In one embodiment of this aspect, the dosages are individual dosage units of Compound (1). In a further embodiment, an individual dosage unit comprises 45 mg to 55 mg of Compound (1). In a further embodiment, an individual dosage unit comprises 48 mg to 52 mg of Compound (1). In still a further embodiment, an individual dosage unit comprises 50 mg of Compound (1). In still a further embodiment, an individual dosage unit comprises 40 mg of Compound (1). In one embodiment, the episodic dosing regimen has a duration of about 2 to about 8 weeks. In another embodiment, the episodic dosing regimen has a duration of about 2 to about 6 weeks. In a further embodiment, the episodic dosing regimen has a duration of about 2 to about 4 weeks. In still a further embodiment, the episodic dosing regimen has a duration of about 2 weeks or 14 days. In yet a further embodiment, the episodic dosing regimen has a duration of 2 weeks. In one embodiment, the major depressive disorder is moderate major depressive disorder. In another embodiment, the major depressive disorder is severe major depressive disorder. In one embodiment, the instruction set is printed on a suitable material. In another embodiment, the individual dosage units are capsules or tablets. In a further embodiment, the individual dosage unit is a capsule. In some embodiments, the individual dosage unit is a capsule of size 1, 2, 3, or 4. In one embodiment, the capsule is size 1.

In some embodiments, the method improves cognitive function in the subject. In some embodiments, the method improves cognitive function in the subject after completing the episodic dosing regimen. In some embodiments, the method provides no cognitive impairment in the subject.

In another aspect, the invention includes a method of treating major depressive disorder in a subject in need thereof, the method comprising the steps of:
(i) performing an initial administration cycle on the subject, wherein the initial administration cycle consists essentially of a dosing period comprising administering once daily to the subject a daily dose comprising about 30 mg or about 50 mg of Compound (1):

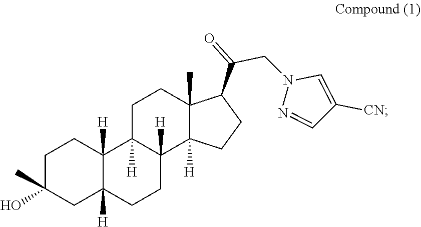

for a time period of two weeks, followed by a non-dosing period comprising a time period of at least 8 weeks where the subject is not administered Compound (1); and
(ii) performing 0, 1, 2, 3, or 4 subsequent administration cycles on the subject, wherein each subsequent administration cycle consists essentially of a dosing period comprising administering once daily to the subject a daily dose comprising about 30 mg or about 50 mg of Compound (1) for a time period of two weeks, followed by a non-dosing period comprising a time period of at least 8 weeks where the subject is not administered Compound (1).

In one embodiment of this aspect, the total time for the initial administration cycle and all subsequent administration cycles is not more than one year. In another embodiment, there are no more than four subsequent administration cycles within the year. In some embodiments there is an 8 week interval between administration of Compound (1) to the subject and re-administration of Compound (1) to the subject. Administration of one or more subsequent administration cycles to a subject who has received an initial administration cycle is interchangeably referred to as re-administration of Compound (1), and also interchangeably referred to as re-treatment. In one embodiment, each subsequent administration cycle is performed in response to a recurrence of depression symptoms after the non-dosing period of the previous administration cycle. In another embodiment, the recurrence of depression symptoms requiring a subsequent administration cycle is determined by an evaluation of the subject using the Hamilton Rating Scale for Depression (HAM-D). Montgomery-Asberg Depression Rating Scale (MADRS), the Patient Health Questionnaire (PHQ-9), or a combination thereof. In another embodiment, the recurrence of depression symptoms requiring a subsequent administration cycle is determined by a HAM-D score of ≥20. In a further embodiment, the recurrence of depression symptoms requiring a subsequent administration cycle is further determined by a PHQ-9 score of ≥10. In still a further embodiment, the recurrence of depression symptoms requiring a subsequent administration cycle is further determined by a MADRS score of ≥28. In one embodiment, the non-dosing period is at least 10 weeks, and no more than 3 subsequent administration cycles are performed on the subject. In another embodiment, the non-dosing period is at least 12 weeks, and no more than 2 subsequent administration cycles are performed on the subject. In another embodiment, the non-dosing period is at least 16 weeks, and no more than 1 subsequent administration cycle is performed on the subject. In another embodiment, the non-dosing period is at least 24 weeks, and no subsequent administration cycles are performed on the subject. In some embodiments, the depression symptoms do not recur. In some embodiments, the initial administration cycle comprises a daily dose of 30 mg of Compound (1). In a further embodiment, each and every subsequent administration cycle comprises a daily dose of 30 mg of Compound (1). In another embodiment, one or more subsequent administration cycles comprise a daily dose of 50 mg of Compound (1), and the rest of the subsequent administration cycles comprise a daily dose of 30 mg of Compound (1). In another embodiment, each and every subsequent administration cycle comprises a daily dose of 50 mg of Compound (1). In some embodiments, the initial administration cycle comprises a daily dose of 50 mg of Compound (1). In a further embodiment, each and every subsequent administration cycle comprises a daily dose of 50 mg of Compound (1). In another embodiment, one or more subsequent administration cycles comprise a daily dose of 30 mg of Compound (1), and the rest of the subsequent administration cycles comprise a daily dose of 50 mg of Compound (1). In another embodiment, each and every subsequent administration cycle comprises a daily dose of 30 mg of Compound (1). In one embodiment, the subject is treatment naïve to any form of medicament for treating depression. In a further embodiment, the subject is primary naïve. In a further embodiment, the subject is secondary naïve. In one embodiment, the subject is currently taking or has recently taken antidepressant medication. In a further embodiment, the subject was on a stable dose of the antidepressant medication for at least 60 days prior to the start of the initial administration period. In one embodiment, the major depressive disorder is moderate major depressive disorder. In another embodiment, the major depressive disorder is severe major depressive disorder. In one embodiment, the subject has been experiencing a major depressive episode over about a 1-year period. In one embodiment, the subject is between about 18 and about 75 years of age. In a further embodiment, the subject is between about 18 and about 65 years of age. In one embodiment, the daily dose of Compound (1) is administered to the subject in the evening. In another embodiment, the daily dose is administered to the subject concurrently with, or immediately after ingestion of food. In one embodiment, the daily dose comprising Compound (1) is in the form of a capsule. In another embodiment, the method further comprises administering to the subject a second therapeutic agent.

### EXAMPLES

### Example 1. A Phase 3, Open-Label, 1-Year Study of the Safety, Tolerability, and Need for Re-Treatment with Compound (1) in Adult Subjects with Major Depressive Disorder

| ABBREVIATION | DEFINITION OR DESCRIPTION |
|---|---|
| AE | Adverse event |
| CGI-I | Clinical Global Impression - Improvement |
| CGI-S | Clinical Global Impression - Improvement |
| C1 | Cycle 1 |
| CI | Confidence Interval |
| C-SSRS | Columbia Suicide Severity Rating Scale |
| ECG | Electrocardiography |
| FAS | Full Analysis Set |
| HAM-D | Hamilton Rating Scale for Depression |
| HAM-A | Hamilton Anxiety Rating Scale |
| HSV | Herpes Simplex Viral |
| ICF | Informed consent form |
| LS Mean | Least Square Mean |
| MADRS | Montgomery - Asberg Depression Rating Scale |
| MDD | Major Depressive Disorder |
| MedDRA | Medical Dictionary for Regulatory Activities |
| MMRM | Mixed effect Model Repeat Measurement |
| PT | Preferred term |
| SAE | Serious adverse event |
| SE | Standard Error |
| SOC | System organ class |
| TEAE | Treatment emergent adverse event |

Primary Objective: To determine the safety and tolerability of initial treatment and re-treatment(s) with Compound (1) in adults with Major Depressive Disorder (MDD) currently experiencing a major depressive episode (MDE) over a 1-year period.

Secondary Objective: To assess the need for re-treatment with Compound (1) following initial treatment in adults with MDD currently experiencing an MDE over a 1-year period, and to assess the response of initial treatment and re-treatment(s) with Compound (1) following an initial 2-week treatment period in adults with MDD currently experiencing an MDE over a 1-year period.

Primary Endpoints: The safety and tolerability of the initial treatment with Compound (1) and re-treatment with Compound (1), as assessed by the incidence and severity of adverse events/serious adverse events; changes from baseline in clinical laboratory measures, vital signs, and electrocardiograms (ECGs); and suicidal ideation and behavior using the Columbia Suicide Severity Rating Scale (C-SSRS) through 1 year. The safety and tolerability of the initial treatment with Compound (1) and re-treatment with Compound (1), as assessed by the incidence and severity of adverse events/serious adverse events; changes from baseline in clinical laboratory measures, vital signs, and electrocardiograms (ECGs); and suicidal ideation and behavior using the Columbia Suicide Severity Rating Scale (C-SSRS)

Secondary Endpoints: The need for re-treatment with Compound (1) as assessed by: time to first re-treatment (Kaplan-Meier curves), number of subjects achieving the requirements for re-treatment, number of re-treatment cycles for each subject, the response of initial treatment and/or re-treatment as assessed by: change from baseline in the 17-item Hamilton Rating Scale for Depression (HAM-D) total score at the end of each 14-day treatment (initial and/or re-treatment) period, HAM-D response at the end of each 14-day treatment (initial and/or re-treatment) period, defined as a ≥50% reduction in HAM-D score from baseline, HAM-D remission at the end of each 14-day treatment (initial and/or re-treatment) period, defined as HAM-D total score≤7, Clinical Global Impression-Improvement (CGI-I) response, defined as “much improved” or “very much improved”, at the end of each 14-day treatment (initial and/or re-treatment) period, change from baseline in Clinical Global Impression-Severity (CGI-S) score at the end of each 14-day treatment (initial and/or re-treatment) period.

Study Population: Subjects were 18-75 years old, diagnosed with major depressive disorder (MDD) with a MADRS total score of ≥28 and a HAM-D total score of ≥20 at Screening and Day 1 (prior to dosing).

Treatment Groups: The 30 mg and 50 mg dose groups were separated by time of enrollment. The study is comprised of two cohorts: one with Compound (1) 30 mg as a starting dose (30 mg Cohort: n=725), an initial cohort with Compound (1) 50 mg as a starting dose (50 mg Cohort: n=199). A patient receiving Compound (1) 30 mg could be dose reduced to 20 mg and patients receiving Compound (1) 50 mg could be dose reduced to 40 mg (based on tolerability). In all cohorts, Compound (1) is self-administered by patients as an oral therapy once nightly with food for 14 days.

### Study Design

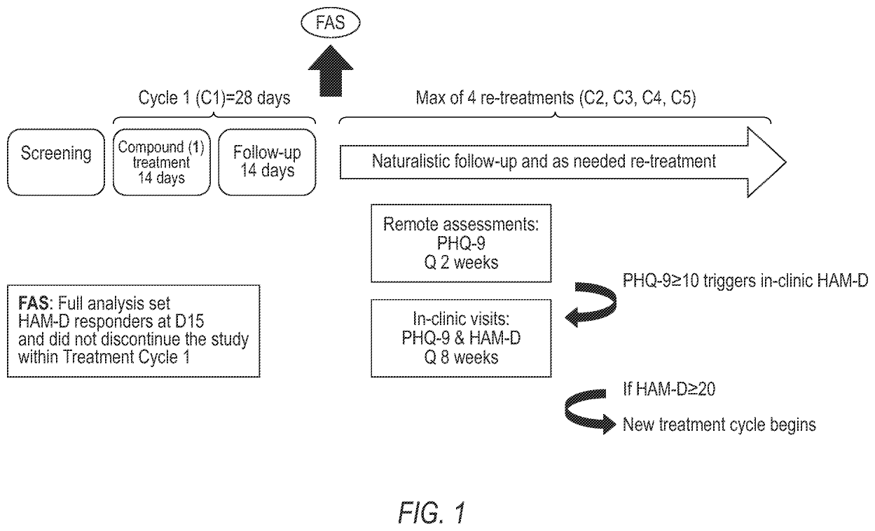

The study has a Screening Period of up to 28 days, a 14-day Treatment Period, and up to 1 year of Follow-up. FIG. 1 is an overview of the study.

The Screening Period begins with the signing of the informed consent form (ICF) at the Screening Visit: the ICF must be signed prior to beginning any screening activities. The diagnosis of MDD must be made according to Structured Clinical Interview for Diagnostic and Statistical Manual of Mental Disorders, Fifth Edition (DSM-5) Clinical Trial Version (SCID-5-CT) performed by a qualified healthcare professional. Subjects will undergo preliminary screening procedures at the Screening Visit to determine eligibility, including completion of the MADRS, HAM-D, and CGI-S.

Antidepressants were permitted provided subjects were on a stable dose for at least 60 days prior to Day 1 and agreed to continue on the stable dose through the follow-up period (Day 42). Initiation of new antidepressants or any other medications that may potentially have an impact on efficacy or safety endpoints were not allowed between screening and completion of the Day 42 assessments.

Eligible subjects were given Compound (1)—to be taken at home in the evening with meal. Subjects who showed reduction of at least 50% in HAM-D total score at Day 15, i.e. responders in Treatment Cycle 1 were allowed to continue in the study; others were terminated after a 14-day follow up (Day 28).

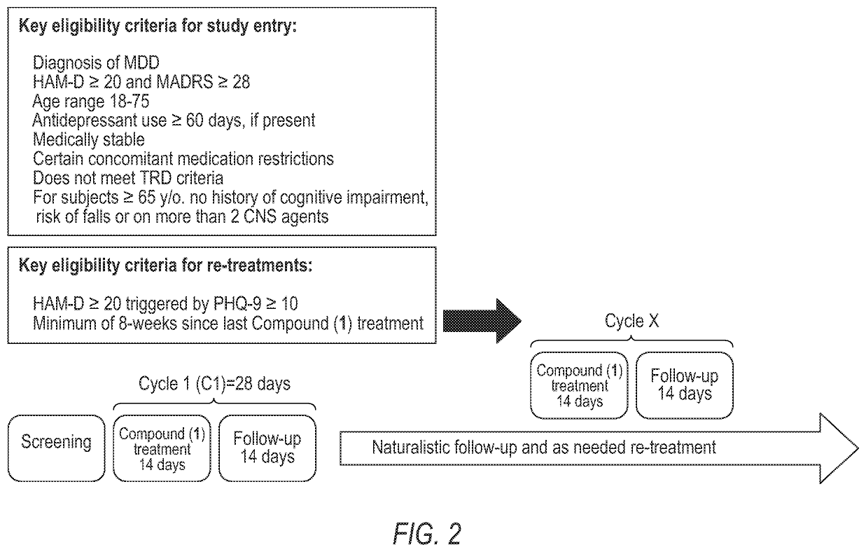

Subjects who continued in the Observation Period were monitored remotely via PHQ-9 assessment every two weeks. If PHQ-9 score was >=10 at any assessment, PHQ-9 assessment was switch to every week until the score came down below 10 or the subject was dosed with Compound (1) in a subsequent cycle, whichever was earlier. A subject was to come back to the site for HAM-D assessment within a week of PHQ-9 score >=10; if the HAM-D total score >=20 and at least 56 days had elapsed since the last dose of Compound (1), another dosing cycle of Compound (1) were to start. If any of these criteria were not met, the subject couldn't be treated with Compound (1) at that time. Without the trigger of PHQ-9>=10 the subject was to have a clinic visit every 8 weeks. A subject could have at most 5 cycles of treatment within a year. FIG. 2 is a depiction of the eligibility criteria of the study.

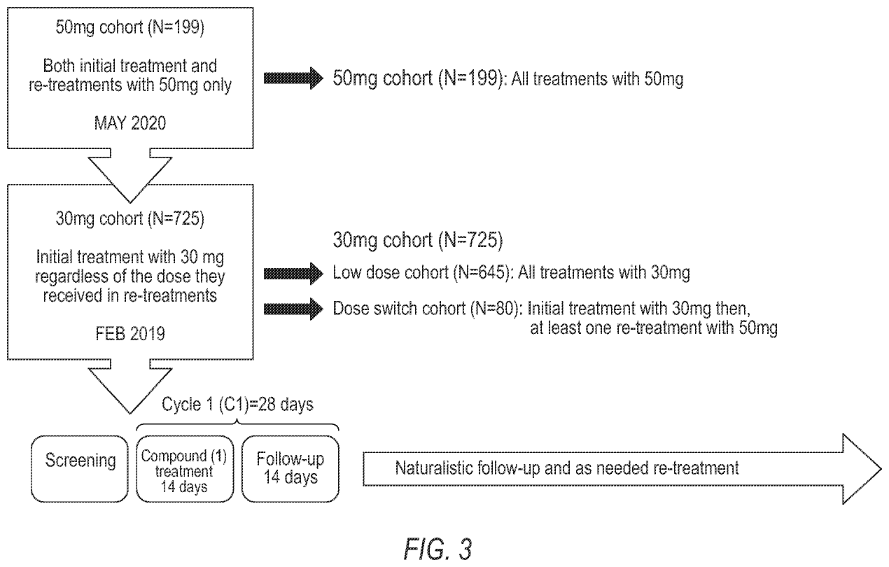

FIG. 3 is a flow chart of the dosing cohorts.

At the time of introduction of 50 mg, subjects who were treated with 30 mg in past cycles were switched to 50 mg in the next treatment cycle.

Dose reduction from 30 mg to 20 mg or 50 mg to 40 mg was allowed in any treatment cycle if the safety and/or tolerability of the subject so warrants. Further, if a subject was dose-reduced from 50 mg to 40 mg in any cycle, for the next treatment cycle the investigator had a choice of starting the subject with 50 mg or 40 mg. Subjects who dose reduced from 30 mg to 20 mg still started with 30 mg in the next treatment cycle.

Antidepressant use could be modified between treatment cycles according to specific rules laid down in the protocol.

Key inclusion criteria: Female and male patients, aged 18-75 years with a diagnosis of MDD with symptoms present for ≥4 weeks, and HAM-D17 total score ≥20 and MADRS total score ≥28 at screening and Day 1 (prior to dosing). Patients taking antidepressants must have been taking these medications at the same dosage for ≥60 days prior to Day 1 with intent to continue through first treatment cycle.

Key exclusion criteria: Active psychosis, attempted suicide or at risk of suicide associated with the current episode of MDD, medical history of bipolar disorder, schizophrenia, and/or schizoaffective disorder, or treatment-resistant depression, which is defined as persistent depressive symptoms despite treatment with adequate doses of antidepressants (excluding antipsychotics) from 2 different classes within the current MDE for at least 4 weeks of treatment.

Determination of Sample Size: The sample size was not based on a formal sample size calculation. The sample size of 900 subjects was chosen in order to have at least 450 subjects complete 24 weeks of the study and at least 150 subjects complete through 56 weeks.

The cohorts pertaining to the different doses that the subjects received were defined as following:
50 mg cohort: This cohort includes subjects who have initiated treatment with 50 mg and received re-treatment with 50 mg only.
30 mg cohort: This cohort includes subjects who have initiated treatment with 30 mg regardless of the dose they have received in re-treatments.
Low dose cohort: This cohort includes subjects who have initiated treatment with 30 mg and received re-treatment with 30 mg only.
Dose switch cohort: This cohort includes subjects who have received initial treatment with 30 mg and at least one subsequent re-treatment with 50 mg.
Study Period X: starts on the date of the first dose of Compound (1) in cycle X and goes up to one day prior to the first dose of the next cycle. In some embodiments, X is any number from 1 to 5.
Treatment Period X: starts on the date of the first dose of Compound (1) in cycle X and goes up to one day after the last dose in cycle X. In some embodiments, X is any number from 1 to 5.
Treatment Cycle X: starts on the date of the first dose of Compound (1) in cycle X and goes up to 14 days after the last dose in cycle X. The treatment cycles may be denoted as C1-C5.
Observation period X: starts the day after Treatment Cycle X ends and goes up to one day prior to the start of next treatment cycle. In some embodiments, X is any number from 1 to 5.

Using the aforementioned, for Study Period 1, the 30 mg Cohort was essentially the same as the combined group of Low Dose Cohort and Dose Switch Cohort. 725 subjects were dosed in the 30 mg Cohort (n=645 in the Low Dose Cohort and n=80 in the Dose Switch Cohort), and 199 subjects were dosed in the 50 mg Cohort. The 30 mg Cohort completed one-year follow-up.

Analysis Sets: The safety set was defined as all subjects who were administered study drug. The period-specific safety set includes subjects in the safety set who were administered study drug in the corresponding treatment cycle. The Full Analysis Set (FAS) was defined as all subjects in the safety set who had HAM-D response at Day 15 in treatment cycle 1 and discontinuation from study date, if it exists, was after the end of treatment cycle 1. (A Treatment Cycle X responder was a safety set subject whose Day 15 HAM-D total score in Treatment Cycle X showing at least 50% reduction from Baseline. If Day 15 HAM-D total score in Treatment Cycle X was missing, the subject was considered a non-responder.)

The Dose Switch Cohort was defined as all subjects in the safety set who received 30 mg in Treatment Cycle 1, Day 1 and received 50 mg on Day 1 in a subsequent re-treatment cycle.

### Statistical Analysis

Whenever possible, data was presented for all cohorts separately, in addition to all cohorts together as Compound (1) Overall Cohort. For Dose Switch Cohort, the data was presented by study period, by the Treatment Cycle X. Day 1 dose received per dose dispensation in the specific period as well as for Compound (1) overall (irrespective of dose received). All displays were presented based on treatment received, with no consideration for planned treatment.

Disposition of subjects, including the number of subjects dosed in each cycle and number of subjects who had been dosed in exactly X number of treatment cycles has been provided. Descriptive statistics of HAM-D total score, HAM-D response and remission for treatment cycle 1 have been provided for subjects dosed in treatment cycle 1 (safety set). The rest of the analyses of HAM-D total score uses FAS for dosed subjects in each treatment cycle. Line plot of LS mean change from baseline in HAM-D total score for each study period is provided, based on a mixed effects model for repeated measures (MMRM) for each study period and each dose cohort separately: the model will include respective period-specific baseline score, anti-depressant use at baseline (Yes or No), assessment time point, as explanatory variables. HAM-D summaries for Dose Switch Cohort by the first dose in each cycle has been provided. In addition, the HAM-D total score summary for subjects dosed in Cycle X for current and past treatment cycles is provided. The response at Day 15 for past cycles for subjects who are non-responders in current cycle is provided. Bar charts for HAM-D response and remission by study period have been provided.

For safety analysis, the period-specific safety set has been used, in addition to Dose Switch Cohort when appropriate. Treatment period is defined as first dose in the study period until last dose date +1 day. Treatment cycle is defined as first dose date in the study period until 14 days after the last dose date. Adverse events have been analyzed by dose cohort for:
1. Overview of treatment emergent adverse events, including number and percentage of subjects with TEAE, TEAE for treatment cycle period, treatment period, post-treatment period, TEAE by maximum severity, TEAE leading to study drug discontinuation or dose reduction, serious TEAE—overall as well as by study period.
2. Treatment cycle period AEs by SOC and PT—by study period, and by first dose of study period using Dose Switch Cohort.
3. Treatment cycle period AEs by SOC/PT and maximum severity—by study period, and by first dose of study period using Dose Switch Cohort.
4. TEAE leading to study drug withdrawal, by SOC/PT.
5. TEAE leading to dose reduction in study drug, by SOC/PT.
6. Treatment cycle period Serious TEAE by SOC/PT—by study period, and by first dose of study period using Dose Switch Cohort.

### Example 2. Results from Example 1—Topline Results

The data used in this Example 2 was data with a cut-off date that was the study completion date of the last subject who started the study with 30 mg dose. All subjects in 50 mg Cohort completed Treatment Cycle 1 of 28 days as of this cut-off date, but some had not been in the study long enough to be eligible for any re-treatment. As of this data cut-off date, only 1 subject in 50 mg Cohort had been dosed in Treatment Cycle 3 (including the initial cycle).

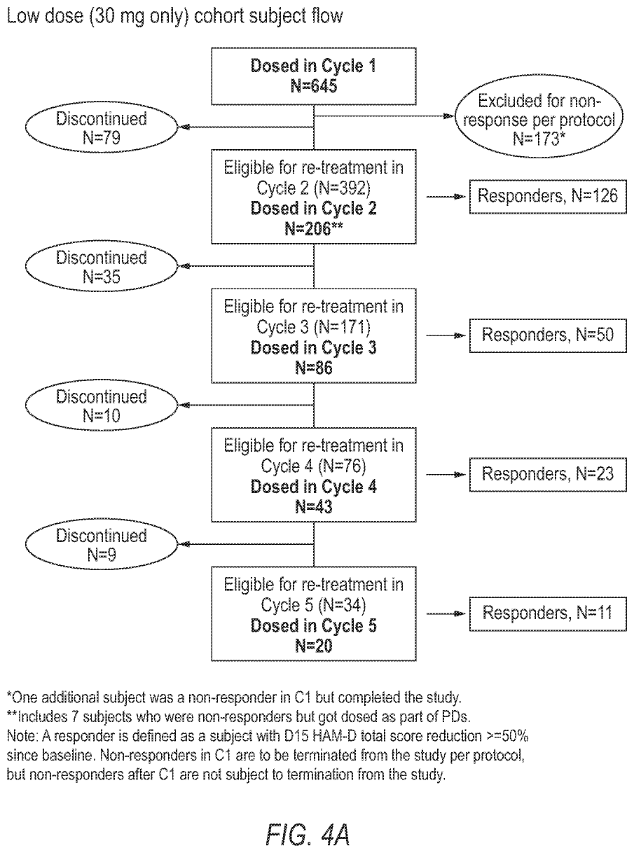

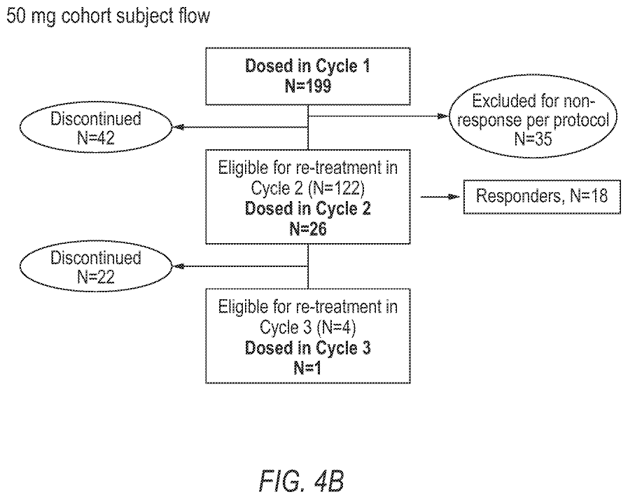

The diagram in FIGS. 4A and 4B provide an overview of the flow of subjects.

### Results

### Primary Endpoint-Safety

Similar proportions of subjects have reported treatment-emergent adverse events (TEAEs) across the dose cohorts: 50 mg Cohort: 62.8% (125/199): 30 mg Cohort: 68.0% (493/725); Low Dose Cohort: 67.6% (436/645); Dose Switch Cohort: 71.3% (57/80)

TEAEs that have happened in more than 5% of subjects in any dose cohort are provided in the table below.

TABLE 1
TEAEs that have happened in more than 5% of subjects

| | | Low Dose | Dose Switch |
|---|---|---|---|
| | 50 mg Cohort | Cohort | Cohort |
| | (N = 199) | (N = 645) | (N = 80) |
| Headache | 20 (10.1%) | 90 (14.0%) | 13 (16.3%) |
| Somnolence | 29 (14.6%) | 76 (11.8%) | 10 (12.5%) |
| Dizziness | 30 (15.1%) | 47 (7.3%) | 7 (8.8%) |
| Sedation | 18 (9.0%) | 29 (4.5%) | 11 (13.8%) |
| Diarrhea | 4 (2.0%) | 43 (6.7%) | 11 (13.8%) |
| Dry Mouth | 7 (3.5%) | 40 (6.2%) | 3 (3.8%) |
| Nausea | 11 (5.5%) | 25 (3.9%) | 3 (3.8%) |
| Upper Respiratory | 1 (0.5%) | 52 (8.1%) | 5 (6.3%) |
| Tract Infection | | | |
| Insomnia | 10 (5.0%) | 31 (4.8%) | 5 (6.3%) |
| Back Pain | 0 | 18 (2.8%) | 5 (6.3%) |
| Arthralgia | 1 (0.5%) | 14 (2.2%) | 5 (6.3%) |
| Fatigue | 5 (2.5%) | 27 (4.2%) | 8 (10.0%) |

The overall safety observations in subjects receiving Compound (1) 50 mg to date is comparable with the safety findings observed with 30 mg, and neither dose deviates from the known safety profile of Compound (1).

The majority of the TEAEs were mild to moderate in all dose cohorts, with similar incidences across all dose cohorts.

TABLE 2
TEAE severity in all dose cohorts

| | | 30 mg cohort | | |
|---|---|---|---|---|
| | 50 mg | Low Dose | Dose Switch | 30 mg |
| | Cohort | Cohort | Cohort | Cohort |
| | (N = 199) | (N = 645) | (N = 80) | (N = 725) |
| Mild | 45 (22.6%) | 187 (29.0%) | 13 (16.3%) | 200 (27.6%) |
| Moderate | 65 (32.7%) | 212 (32.9%) | 36 (45.0%) | 248 (34.2%) |
| Severe | 15 (7.5%) | 37 (5.7%) | 8 (10.0%) | 45 (6.2%) |

The percent of subjects reporting TEAEs leading to study drug discontinuation was 6.5% ( 13/199) in the 50 mg cohort, 2.8% ( 18/645) in the Low dose cohort and 1.3% ( 1/80) in the Dose switch cohort.

The percent of subjects reporting TEAEs leading to withdrawal from study was 7.0% ( 14/199) in the 50 mg cohort, 4.8% ( 31/645) in the Low dose cohort and 1.3% ( 1/80) in the Dose switch cohort.

The percent of subjects reporting TEAEs leading to dose reduction was, in the 50 mg cohort: 17.1% ( 34/199), all but 2 in Cycle 1, 2/26 in Cycle 2. In the Low dose cohort: overall 5.0% ( 32/645): 3.4% ( 22/645) in Cycle 1, 4.4% ( 9/206) in Cycle 2, 2.3% ( 2/86) in Cycle 3, 0 ( 0/43) in Cycle 4 and 10% ( 2/20) Cycle 5 respectively. In the Dose switch cohort: overall 15.0% ( 12/80): 2.5% ( 3/80) each in Cycle 1 and Cycle 2 while on 30 mg; 4.2% ( 3/71) in Cycle 3, 3.8% ( 2/53) in Cycle 4, 17.4% ( 4/23) in Cycle 5 while switched to 50 mg. Somnolence, Dizziness, Sedation and Headache are the most common AEs that led to dose reduction.

### Secondary Endpoint-Efficacy

TABLE 3
Change in HAM-D total score at Day 15 (safety set)

| | All Safety Set* | | | Baseline HAM-D >=24 | | Baseline HAM-D >=26 | |
|---|---|---|---|---|---|---|---|
| | 50 mg Cohort | | 30 mg | 50 mg | 30 mg Cohort | 50 mg Cohort | 30 mg Cohort |
| | (N = 199) | | Cohort | Cohort | (N = 458) | (N = 85) | (N = 327) |
| n at Day 15 | 185 | | 687 | 118 | 433 | 80 | 308 |
| Mean ± SD CFB | −16.0 ± 6.04 | | −15.2 ± 7.07 | −17.4 ± 6.05 | −16.9 ± 7.34 | −18.2 ± 5.89 | −17.6 ± 7.77 |
| Mean ± SD % | −63.6 ± 22.70 | | −60.0 ± 25.61 | −64.1 ± 21.73 | −60.9 ± 25.50 | −64.3 ± 20.17 | −60.6 ± 26.28 |
| CFB | | | | | | | |
| Response | 149 | (80.5%) | 505 (73.5%) | 94 (79.7%) | 319 (73.7%) | 64 (80.0%) | 220 (71.4%) |
| Remission | 80 | (43.2%) | 276 (40.2%) | 44 (37.3%) | 164 (37.9%) | 27 (33.8%) | 113 (36.7%) |

*All subjects enrolled (HAM-D >=20, MADRS >=28) and dosed

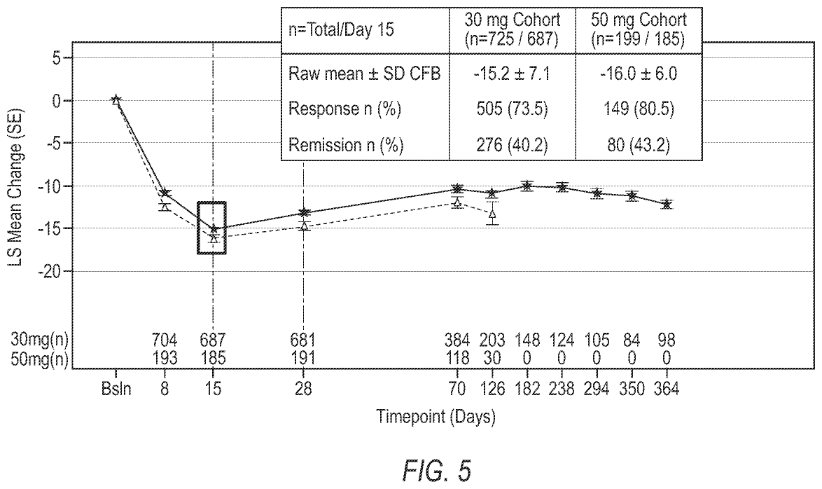

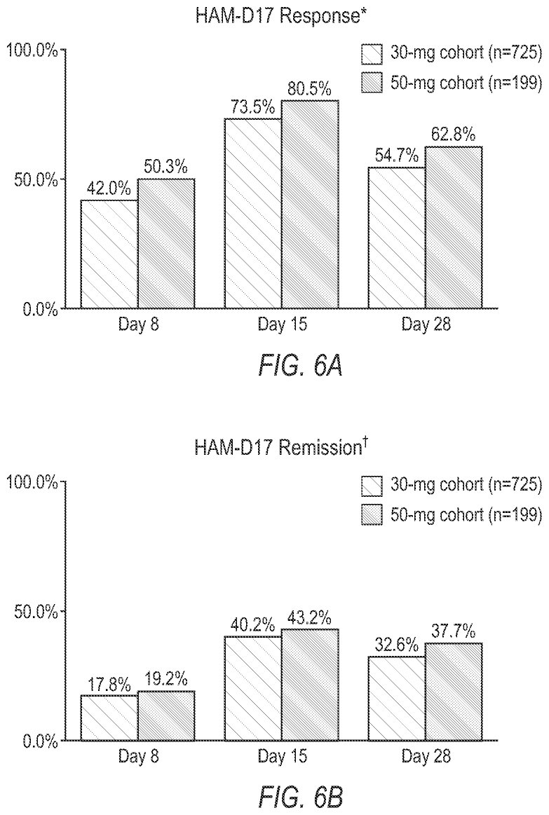

At Day 15, for the 30 mg Cohort, the mean change from baseline was −15.2±7.1 (n=687); 505 (73.5%) patients achieved response and 276 (40.2%) achieved remission (HAM-D≤7). At Day 15 of the initial treatment course in the 50 mg Cohort, the mean HAM-D change from baseline was −16±6.0; 149/185 (80.5%) achieved response and 80/185 (43.2%) achieved remission. The Change in HAM-D total score over Time—Study period 1 (safety set) is shown in FIG. 5. Initial HAM-D Response and Remission are shown in FIGS. 6A and 6B, respectively.

Table 4 shows a summary of re-treatments in the safety set.

TABLE 4
Summary of re-treatments (safety set)

| | 50 mg | 30 mg | |
|---|---|---|---|
| | Cohort | Cohort | Low Dose |
| | (N = 199) | (N = 725) | (N = 645) |
| Number of Subjects | 146 | 489 | 409 |
| completing C1 as responder | | | |
| (FAS) | | | |
| Eligible to be retreated in at | 122 | 472 | 392 |
| least one more cycle [1] | | | |
| Qualified to be retreated in at | 36 (29.5%) | 305 (64.6%) | 225 (57.4%) |
| least 1 more cycle [2, 3] | | | |
| Dosed in at least 1 re- | 26 (21.3%) | 279 (59.1%) | 199 0.8%) |
| treatment cycle [3] | | | |

[1] Eligible for re-treatment means that subject completed treatment cycle 1 and did not discontinue the study within 56 days after the last dose date.
[2] Qualified for re-treatment means that subject was eligible for re-treatment and had a HAM-D total score >= 20 before re-treatment.
[3] Percentages use the denominator of number of subjects from Eligible for Re-treatment.

TABLE 5
Number (%) of Subjects by re-treatment Cycles

| | | 30 mg Cohort | | |
|---|---|---|---|---|
| | 50 mg Cohort | Low Dose | Dose Switch | 30 mg cohort |
| | (N = 146) | (N = 409) | (N = 80) | (N = 489) |
| No re-treatment (Cycle 1) | 120 (82.2%) | 210 (51.3%) | 0 | 210 (42.9%) |
| Only 1 re-treatment (Cycle 2) | 25 (17.1%) | 116 (28.4%) | 9 (11.3%) | 125 (25.6%) |
| Only 2 re-treatment (Cycle 3) | 1 (0.7%) | 40 (9.8%) | 18 (22.5%) | 58 (11.9%) |
| Only 3 re-treatment (Cycle 4) | NA | 23 (5.6%) | 30 (37.5%) | 53 (10.8%) |
| Only 4 re-treatment (Cycle 5) | NA | 20 (4.9%) | 23 (28.8%) | 43 (8.8%) |

As shown in Table 5, the majority of patients (68.5%) received only 1 or 2 treatments with Compound (1) over the 12-month follow-up period in the 30 mg Cohort.

The average (range) number of re-treatments over the 1-year follow-up, per subject, was 0.8 (0-4) for the Low Dose Cohort, 2.8 (1-4) for Dose Switch Cohort. Altogether, in the 30 mg Cohort, average number of re-treatments was 1.2 (0-4). Re-treatment rates were similar with and without pre-existing antidepressant therapy. At the time of this reporting, no subjects in the 50 mg Cohort had been in the study for longer than 6 months, with majority (n=114) having been followed for less than 3 months, and only 1 subject had received more than one re-treatment.

TABLE 6
Time lag between re-treatments (Days)

| | | Number of | | | |
|---|---|---|---|---|---|
| | | Subjects | | | |
| | | Dosed at | | | Range |
| | | Each Cycle | Mean (SD) | Median | (Min-Max) |
| Cycle | 50 mg | 26 | 70.5 (18.52) | 62.5 | 58-122 |
| [symbol] | Low Dose | 199 | 99.5 (52.15) | 78 | 58-297 |
| Cycle | 50 mg | 1 | 58 (NA) | 58 | 58-58 |
| [symbol] | Low Dose | 83 | 87.2 (34.97) | 72 | 52-217 |
| Cycle | 50 mg | NA | NA | NA | NA |
| [symbol] | Low Dose | 43 | 72.1 (17.67) | 65 | 57-113 |
| Cycle | 50 mg | NA | NA | NA | NA |
| [symbol] | Low Dose | 20 | 63.2 (7.42) | 60.5 | 58-86 |

[symbol]

indicates data missing or illegible when filed

Time lag between re-treatment (Days) by Use of Antidepressant at Baseline in the Previous Treatment Cycle is shown in Table 7. The use of Antidepressant did not make much difference in the length of time between re-treatments.

TABLE 7
Antidepressant use at baseline in the
previous cycle (Low Dose Cohort)

| | ATD Use at Baseline of Cycle X − 1= | | | |
|---|---|---|---|---|
| | Yes | | No | |
| Dosed in Cycle X, N= | N | Mean (SD) | N | Mean (SD) |
| Cycle 2, N = 199 | 75 | 103.3 (56.26) | 124 | 97.2 (49.59) |
| Cycle 3, N = 83 | 38 | 85.9 (32.14) | 45 | 88.4 (37.52) |
| Cycle 4, N = 43 | 20 | 71.1 (17.21) | 23 | 73.0 (18.40) |
| Cycle 5, N = 20 | 10 | 64.5 (10.09) | 10 | 61.9 (3.28) |

The response rate by Treatment Cycle in the Low Dose Cohort was as follows: Treatment Cycle 2: 59.3% ( 118/199) total response rate. Treatment Cycle 3: 57.8% ( 48/83) total response rate, out of which 75% ( 36/48) subjects were responders in Treatment Cycle 2 and remained responders in Treatment Cycle 3. 25% ( 12/483) were non-responders in Treatment Cycle 2 who responded on re-treatment. Treatment Cycle 4: 51.2% ( 22/43) total response rate, out of which 81.8% ( 18/22) subjects were responders in Treatment Cycle 3, 63.6% ( 14/22) subjects were responders in Treatment Cycle 2 and remained responders in Treatment Cycle 4. 18.2% ( 4/22) subjects were non-responders in Treatment Cycle 3 and 36.4% ( 8/22) were non-responders in Treatment Cycle 2 who responded on re-treatment in Treatment Cycle 4. Treatment Cycle 5: 50.0% ( 10/20) total response rate, out of which 90% ( 9/10) in Treatment Cycle 4, 80% ( 8/10) in Treatment Cycle 3 and 70% ( 7/10) subjects in Treatment Cycle 2 were responders and remained responders in Treatment Cycle 5. 1% ( 1/10), 20.0% ( 2/10) and 30% ( 3/10) subjects were non-responders in Treatment Cycles 4, Treatment Cycle 3 and Treatment Cycle 2, respectively, who responded on re-treatment in Treatment Cycle 5.

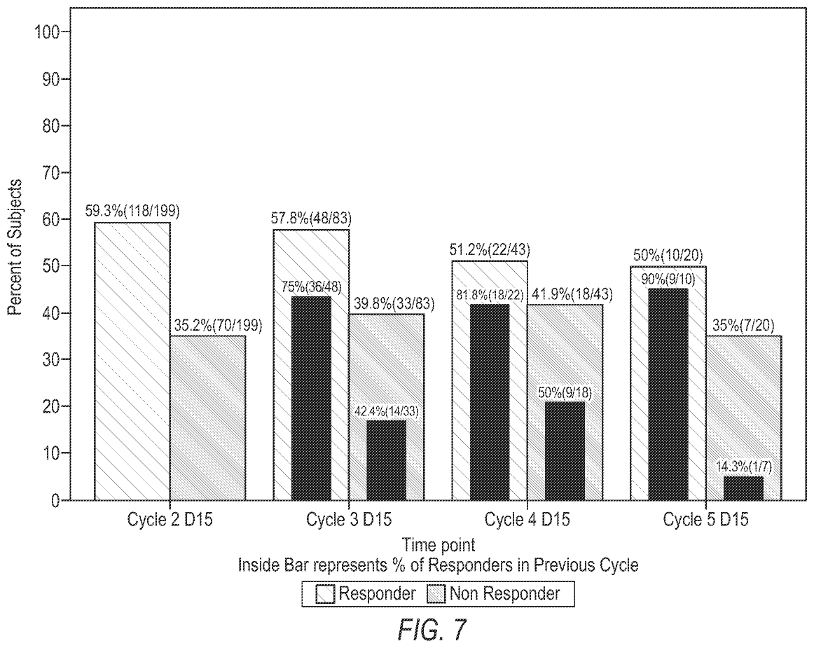

FIG. 7 provides a bar graph of the HAM-D Response/Non-response at Day 15 by Treatment Cycle, Low Dose Cohort only (FAS). Inside bars represent percent of responders in previous cycle.

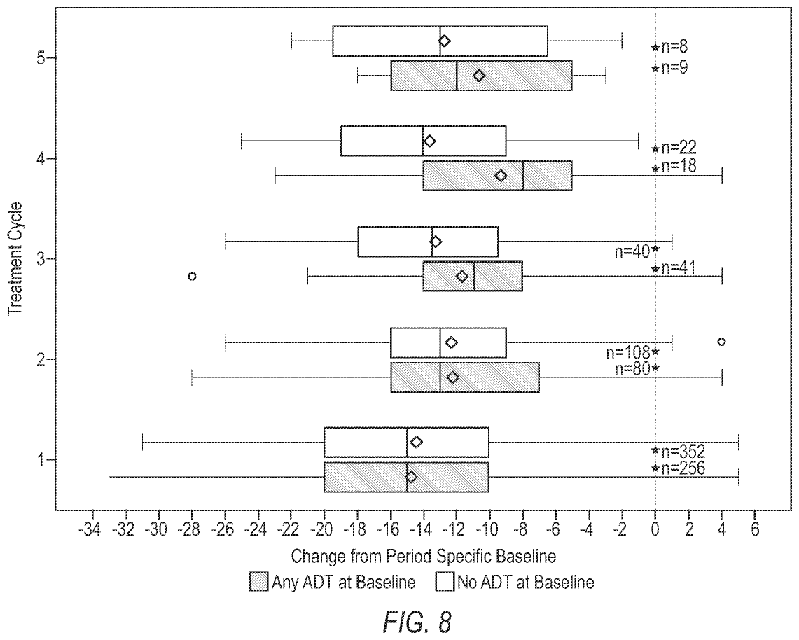

FIG. 8 provides a box plot of Change from Period-Specific Baseline in HAM-D Total Score at Day 15 for in Each Treatment Cycle, by Antidepressant Use (Yes/No) at Period-Specific Baseline-Low Dose Cohort (safety set for Treatment Cycle 1, FAS for Treatment Cycles 2-5).

### Example 3. Results from Example 1—Full Results Set

### Subject Disposition

The data used in this Example 3 was data from the study of Example 1 with a cut-off date that was the study completion date of the last subject who started the study with 30 mg dose. This example presents additional results from Example 2.

A total of 924 subjects were treated with Compound (1) in Treatment Cycle 1 (C1), wherein 725 subjects were treated in the 30 mg Cohort (645 subjects in the Low Dose Cohort and 80 subjects in the Dose Switch Cohort), and 199 subjects were treated in the 50 mg Cohort.

Out of the 725 subjects initially treated with 30 mg, 202 (31.3%) subjects were prematurely withdrawn or withdrew from the study during Treatment Cycle 1. Out of 202 subjects, 146 (72.3%) discontinued from study due to not meeting 50% reduction in HAM-D at Day 15. 523 (72.1%) subjects completed Treatment Cycle 1.

As shown in Table 8, out of the 725 subjects initially treated with 30 mg, 413 (57%) subjects had prematurely withdrawn from the study in 30 mg Cohort before the completion of one year. Out of these 413 subjects, 173 (41.9%) withdrew due to not meeting the responder criterion in Treatment Cycle 1. 146 out of these 173 subjects discontinued within Treatment Cycle 1 as they should have following the protocol, 27 more subjects discontinued the study for being a non-responder in Treatment Cycle 1 after completing Treatment Cycle 1, bringing the total to 173).

Out of the 725 subjects initially treated with 30 mg, 489 (67.4%) subjects out of 725 subjects-409 subjects in the Low Dose Cohort and 80 subjects in the Dose Switch Cohort were responders in Treatment Cycle 1 and completed Treatment Cycle 1, hence were in FAS.

Out of the 80 subjects in the Dose Switch Cohort, 10 subjects switched to 50 mg in Treatment Cycle 2, 21 in Treatment Cycle 3, 38 in Treatment Cycle 4, and 11 in Treatment Cycle 5. Two of the subjects who switched to 50 mg in Treatment Cycle 3 started Treatment Cycle 4 with a reduced dose of 40 mg. Four subjects in this cohort withdrew from the study in a cycle when they switched to 50 mg−1 discontinued study treatment during Treatment Cycle 2 and withdrew due to Physician's decision: 1 discontinued treatment during Treatment Cycle 5, and withdrew due to adverse event: 1 withdrew consent during Treatment Cycle 4, and one withdrew after completing Treatment Cycle 5, but unable to complete end of study visit due to schedule conflict.

Out of the 199 subjects dosed in the 50 mg Cohort, 64 (32.2%) subjects had withdrawn from the study until the data cut-off date. Out of 64 subjects withdrawn from the study, 35 (54.7%) withdrew due to not meeting the 50% reduction in HAM-D at Day 15 and 14 (21.9%) withdrew for adverse event. Out of 199 subjects initially treated with 50 mg, 146 (73.4%) were counted in FAS (responders in Treatment Cycle 1 and did not discontinue study in Treatment Cycle 1).

TABLE 8
Disposition - Treatment Cycle 1

| | Low Dose Cohort | 50 mg Cohort |
|---|---|---|
| | (n = 645) | (n = 199) |
| Dosed - n | 645 | 199 |
| Withdrawn (Treatment Cycle 1) | 202 (31.3%) | 48 (24.1%) |
| Exit for non-response* | 146 (22.6%) | 31 (15.6%) |
| Adverse event | 15 (2.3%) | 11 (5.5%) |
| Lost to follow-up | 15 (2.3%) | 2 (1%) |
| Withdrawal by subject | 13 (2%) | 3 (1.5%) |
| Non-compliance | 3 (0.5%) | 1 (0.5%) |
| Protocol deviation | 4 (0.6%) | — |
| Physician decision | 3 (0.5%) | — |
| Other | 2 (0.3%) | — |
| Sponsor decision | 1 (0.2%) | |

### Demographics and Baseline Characteristics

The demographic and baseline characteristics of the subjects entering the study were well balanced between the 30 mg Cohort and the 50 mg Cohort. Overall, about 68% were female, 81% were white and average age was 45 years (SD: 14.13 years). About 42% of subjects used antidepressant at baseline. 82% subjects had baseline HAMD≥22.

TABLE 9
Demographics and Baseline Characteristics (safety set)

| | 50 mg Cohort | 30 mg Cohort |
|---|---|---|
| | (N = 199) | (N = 725) |
| Age (years), mean (SD) | 45.0 (14.08) | 45.0 (14.16) |
| Sex, n (%) | | |
| Female | 137 (68.8%) | 489 (67.4%) |
| Male | 62 (31.2%) | 236 (32.6%) |
| Race, n(%) | | |
| White | 175 (87.9%) | 571 (78.8%) |
| Black/African American | 10 (5.0%) | 115 (15.9%) |
| Other | 14 (7.0%) | 39 (5.4%) |
| Ethnicity | | |
| Hispanic/Latino | 44 (22.1%) | 176 (24.3%) |
| Not Hispanic/Latino | 155 (77.9%) | 549 (75.7%) |
| Antidepressant Use at Baseline, n (%) | | |
| Yes | 81 (40.7%) | 304 (41.9%) |
| No | 118 (59.3%) | 421 (58.1%) |
| Baseline BMI (kg/m²), mean (SD) | 29.3 (5.69) | 30.2 (6.75) |
| Baseline HAM-D Total Score, | 25.1 (3.29) | 25.3 (4.09) |
| mean (SD) | | |

TABLE 10
Treatment Completion and Study Participation

| | | | |
|---|---|---|---|
| | 50 mg | | |
| | Cohort | Low Dose | Dose Switch |
| Number of Subjects | (N = 199) | (N = 645) | (N = 80) |

Completing the Latest Treatment Cycle

| | | | |
|---|---|---|---|
| Cycle 1 | 139 | 259 | 1 |
| Cycle 2 | 12 | 99 | 8 |
| Cycle 3 | | 43 | 19 |
| Cycle 4 | | 22 | 30 |
| Cycle 5 | | 20 | 22 |

Length of Study Participation

| | | | |
|---|---|---|---|
| Number of Subjects in the study | 114 | 260 | 0 |
| for less than 3 months | | | |
| Number of Subjects in the study | 85 | 81 | 0 |
| between 3 and 6 Months | | | |
| Number of Subjects in the study | | 41 | 0 |
| between 6 and 9 Months | | | |
| Number of Subjects in the study | | 164 | 45 |
| between 9 and 12 Months | | | |
| Number of Subjects completing | | 99 | 35 |
| 12 Months | | | |

### Efficacy Results

HAM-1) Total Score

TABLE 11
Mean change and percent change in HAM-D Total
Score for Study Period 1 Only (safety set)

| | 50 mg Cohort (N = 199) | | | 30 mg Cohort (N = 725) | | |
|---|---|---|---|---|---|---|
| | | Mean CFB ± | Mean % CFB ± | | Mean CFB ± | Mean % CFB ± |
| Visit | N | SD [1] | SD | N | SD [1] | SD |
| Baseline | 199 | 25.1 ± 3.29 | | 725 | 25.3 ± 4.09 | |
| Day 8 | 193 | −12.3 ± 5.79 | −49.2 ± 22.41 | 704 | −10.9 ± 6.75 | −42.9 ± 25.68 |
| Day 15 | 185 | −16.0 ± 6.04 | −63.6 ± 22.70 | 687 | −15.2 ± 7.07 | −60.0 ± 25.61 |
| Day 28 | 191 | −14.6 ± 6.75 | −58.3 ± 26.05 | 681 | −13.3 ± 8.07 | −51.9 ± 29.96 |
| Day 70 | 118 | −13.6 ± 7.45 | −53.7 ± 28.40 | 384 | −12.1 ± 9.12 | −46.6 ± 34.25 |
| Day 126 | 30 | −16.3 ± 9.06 | −65.2 ± 33.96 | 203 | −15.7 ± 8.07 | −62.3 ± 30.36 |

[1] Baseline row represents HAM-D total score mean ± SD

FIG. 5 provides line plots of LS Mean (±SE) Change from Baseline in HAM-D Total Score for Study Period 1.

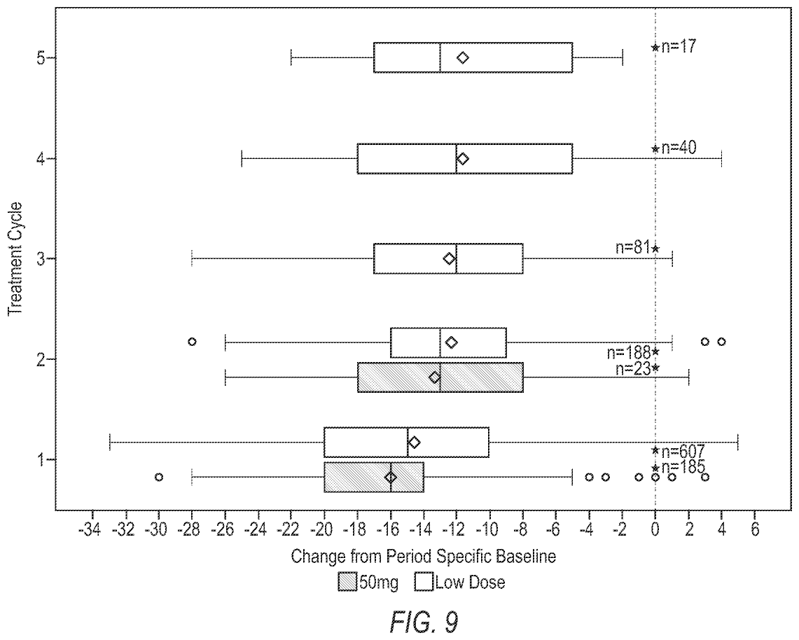

FIG. 9 provides box plots of Change from Baseline HAM-D Total Score at Day 15 in Each Treatment Cycle (safety set for Treatment Cycle 1, FAS for C2-C5).

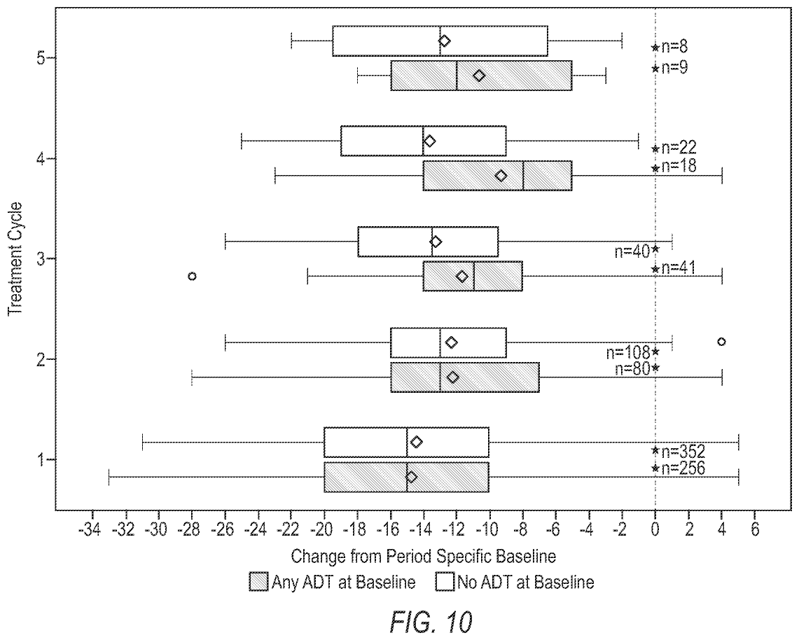

FIG. 10 provides a box Plot of Change from Period-Specific Baseline in HAM-D Total Score at Day 15 for in Each Treatment Cycle, by Antidepressant Use (Yes/No) at Period-Specific Baseline-Low Dose Cohort (safety set for Treatment Cycle 1, FAS for C2-5).

### 2 HAM-D Response

TABLE 12
HAM-D Response for Study period 1 only (safety set)

| | 50 mg Cohort | 30 mg Cohort |
|---|---|---|
| Visit | (N = 199) | (N-725) |
| Day 8 | 97/193 (50.3%) | 295/704 (41.9%) |
| Day 15 | 149/185 (80.5%) | 505/687 (73.5%) |
| Day 28 | 120/191 (62.8%) | 372/681 (54.6%) |
| Day 70 | 69/118 (58.5%) | 193/384 (50.3%) |
| Day 126 | 22/30 (73.3%) | 144/203 (70.9%) |

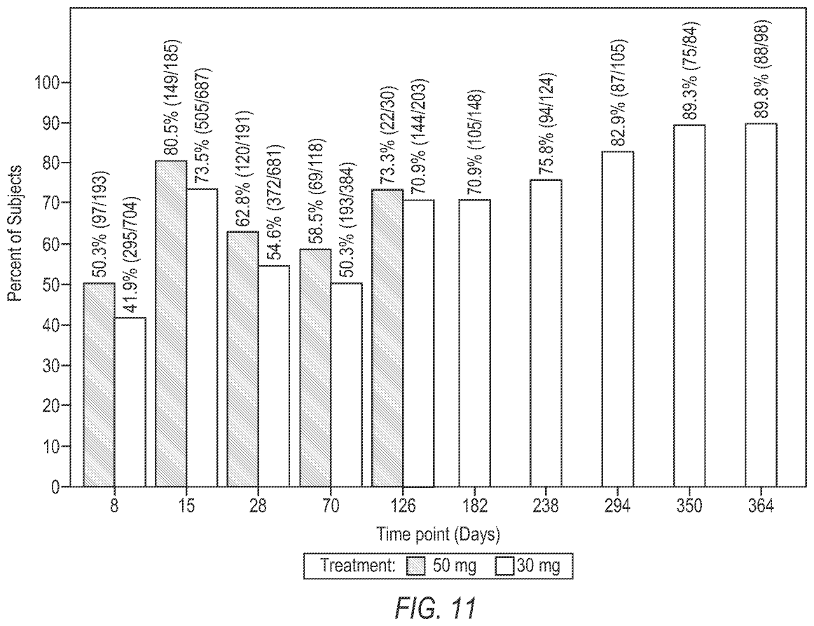

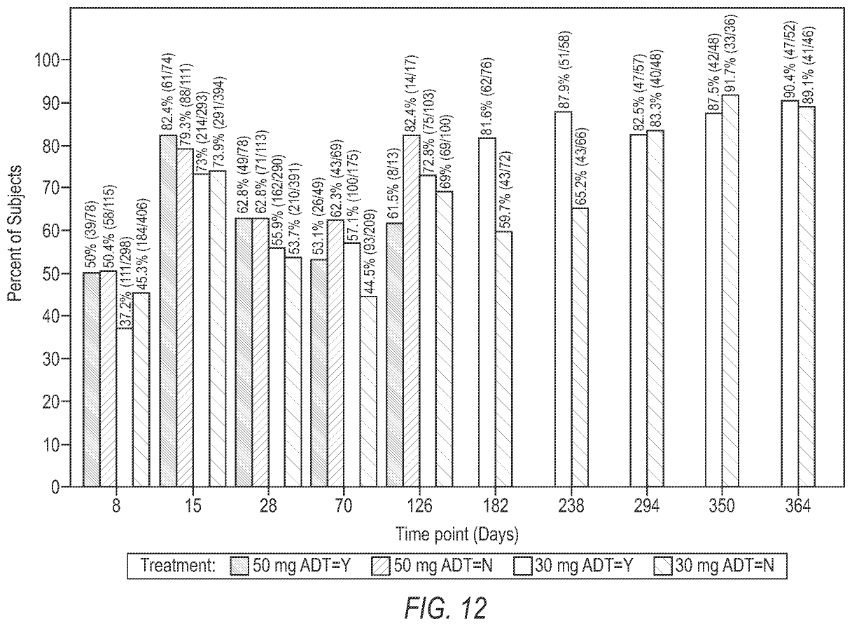

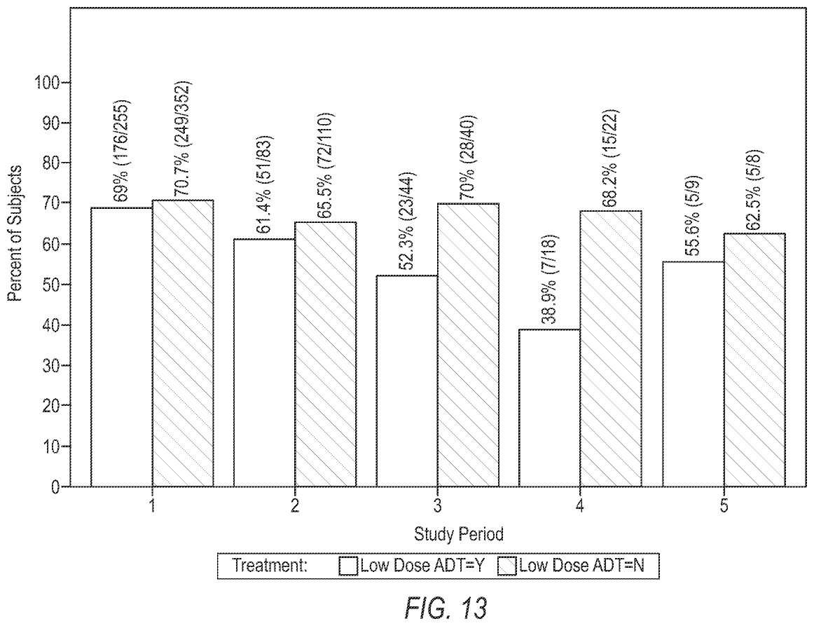

FIG. 11 provides a bar chart of HAM-D Response over time—Study Period 1 (safety set). FIG. 12 provides a bar chart of HAM-D Response over Time in Study Period 1, by Antidepressant Use at Baseline (safety set). FIG. 13 provides a bar chart of HAM-D Response at Day 15 in Each Treatment Cycle, by Antidepressant Use at Period-Specific Baseline-Low Dose Cohort (safety set).

### 3 HAM-D Remission

TABLE 13
HAM-D Remission for Study period 1 only (safety set).

| | 50 mg Cohort | 30 mg Cohort | |
|---|---|---|---|
| Visit | (N = 199) | (N = 725) | |
| Day 8 | 37/193 (19.2%) | 125/704 | (17.8%) |
| Day 15 | 80/185 (43.2%) | 276/687 | (40.2%) |
| Day 28 | 72/191 (37.7%) | 222/681 | (32.6%) |
| Day 70 | 41/118 (34.7%) | 115/384 | (30.0%) |
| Day 126 | 19/30 (63.3%) | 96/203 | (47.3%) |

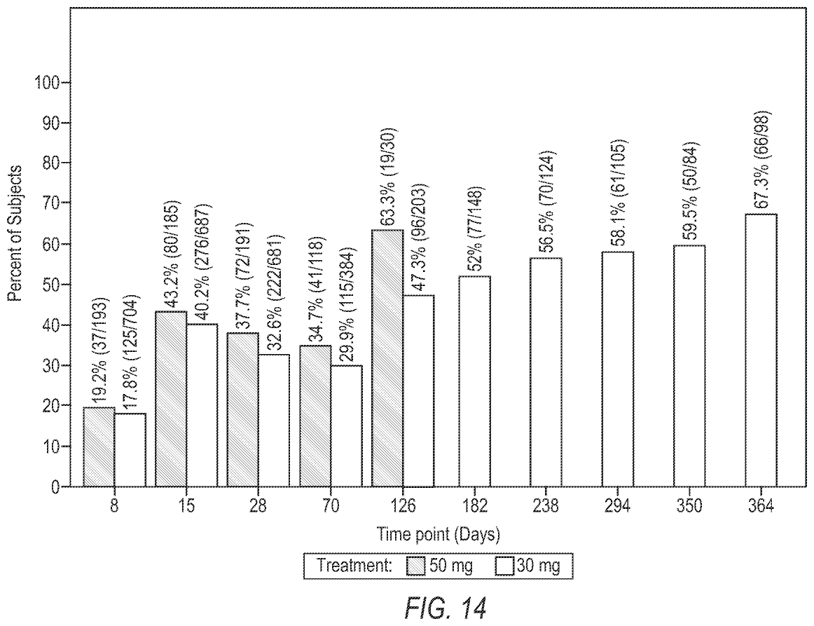

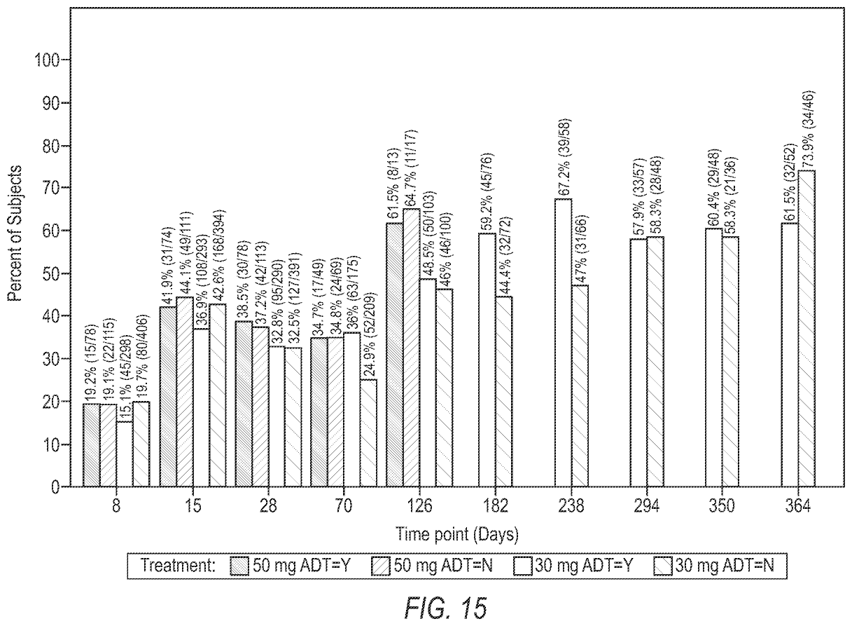

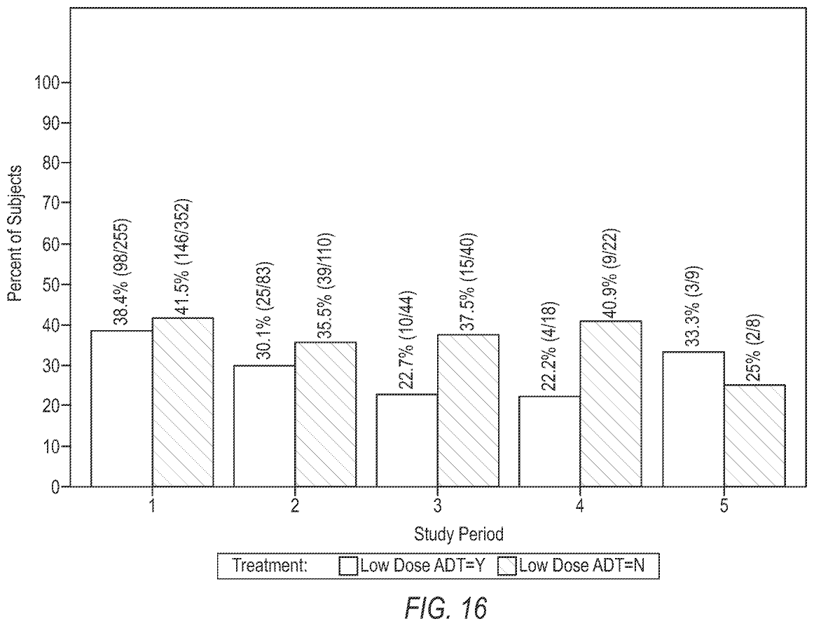

FIG. 14 provides a bar chart of HAM-D Remission Over Time—Study Period 1 (safety set). FIG. 15 provides a bar chart of HAM-D Remission over Time in Study Period 1, by Antidepressant Use at Baseline (safety set). FIG. 16 provides a bar chart of HAM-D Remission at Day 15 in Each Treatment Cycle, by Antidepressant Use at Period-Specific Baseline-Low Dose Cohort.

### 4 Subjects Who had Baseline HAM-D Score ≥24 (Total Score, Response, and Remission)

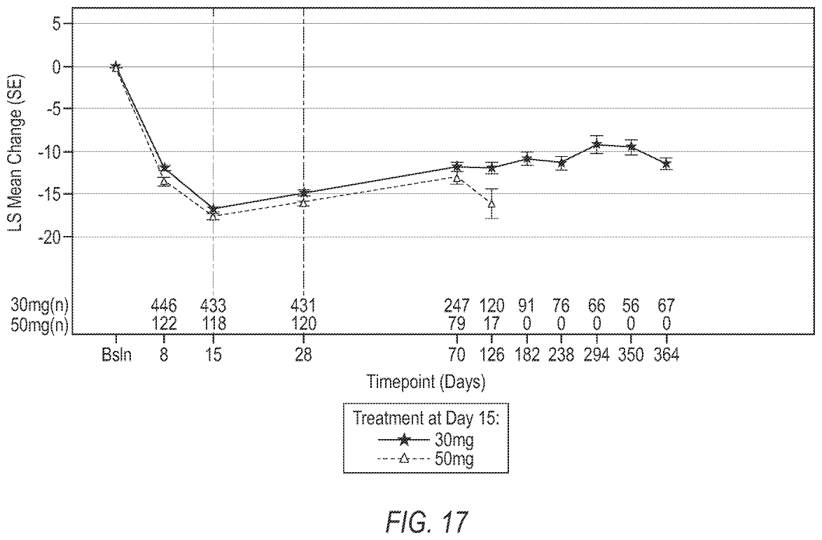

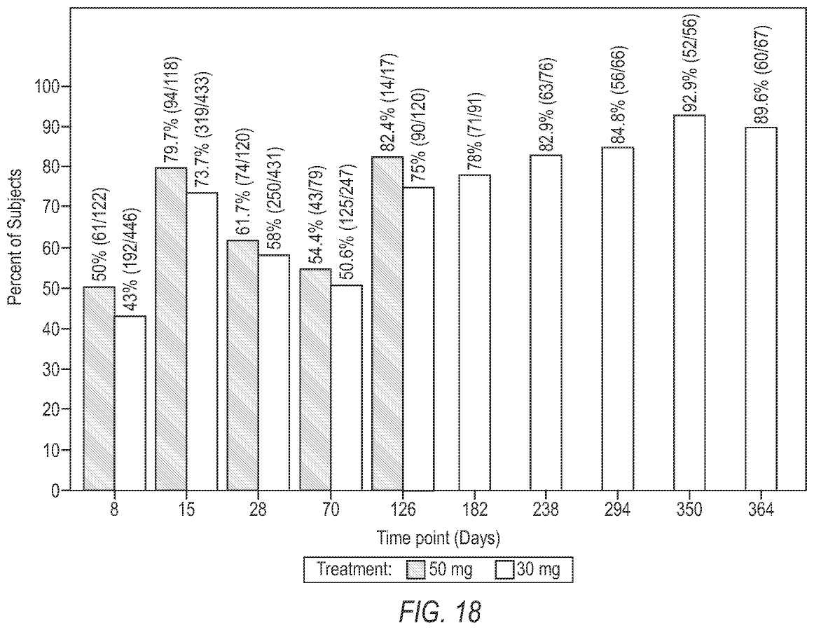

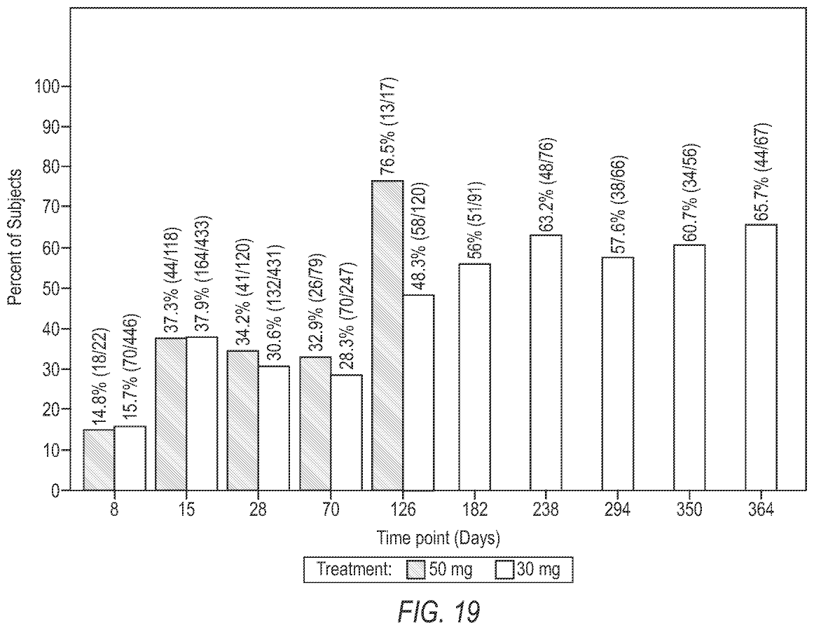

Tables 14, 15, and 16 show HAM-D total score, response, and remission, respectively at various time points of subjects who had a baseline HAM-D score ≥24. FIG. 17 provides line plots of LS mean (±SE) change from baseline for subjects who had baseline HAM-D score ≥24—Study Period 1 (safety set). FIG. 18 and FIG. 19 provide a bar chart of HAM-D Response and Remission, respectively, for Subjects Who had Baseline HAM-D Score ≥24—Study Period 1 (safety set).

TABLE 14
Mean change and percent change in HAM-D total score for subjects
who had baseline HAM-D score ≥24 - Study Period 1 (safety set)

| | 50 mg Cohort (N = 127) | | | 30 mg Cohort (N = 458) | | |
|---|---|---|---|---|---|---|
| | | Mean CFB ± | Mean % CFB ± | | Mean CFB ± | Mean % CFB ± |
| Visit | N | SD [1] | SD | N | SD [1] | SD |
| Baseline | 127 | 27 ± 2.43 | | 458 | 27.7 ± 3.08 | |
| Day 8 | 122 | −13.3 ± 5.87 | −49.3 ± 21.32 | 446 | −12.0 ± 7.17 | −43.3 ± 25.63 |
| Day 15 | 118 | −17.4 ± 6.05 | −64.1 ± 21.73 | 433 | −16.9 ± 7.34 | −60.9 ± 25.50 |
| Day 28 | 120 | −15.7 ± 7.17 | −58.0 ± 25.97 | 431 | −15.0 ± 8.27 | −53.9 ± 28.59 |
| Day 70 | 79 | −14.4 ± 7.93 | −53.0 ± 28.53 | 247 | −13.8 ± 9.39 | −49.2 ± 32.86 |
| Day 126 | 17 | −19.9 ± 8.10 | −74.8 ± 27.94 | 120 | −18.2 ± 7.88 | −66.0 ± 28.29 |

[1] Baseline row represents HAM-D total score mean ± SD

TABLE 15
HAM-D Response for Subjects Who had Baseline HAM-D
Score ≥24 for Study period 1 only (safety set).

| | 50 mg Cohort | 30 mg Cohort |
|---|---|---|
| Visit | (N = 127) | (N = 458) |
| Day 8 | 61/122 (50.0%) | 192/446 (43.0%) |
| Day 15 | 94/118 (79.7%) | 319/433 (73.7%) |
| Day 28 | 74/120 (61.7%) | 250/431 (58.0%) |
| Day 70 | 43/79 (54.4%) | 125/247 (50.6%) |
| Day 126 | 14/17 (82.4%) | 90/120 (75.0%) |

TABLE 16
HAM-D Remission for Subjects Who had Baseline HAM-D
Score ≥24 for Study period 1 only (safety set).

| | 50 mg Cohort | 30 mg Cohort |
|---|---|---|
| Visit | (N = 127) | (N = 458) |
| Day 8 | 18/122 (14.8%) | 70/446 (15.7%) |
| Day 15 | 44/118 (37.3%) | 164/433 (37.9%) |
| Day 28 | 41/120 (34.2%) | 132/431 (30.6%) |
| Day 70 | 26/79 (32.9%) | 70/247 (28.3%) |
| Day 126 | 13/17 (76.5%) | 58/120 (48.3%) |

### 5 Subjects Who had Baseline HAM-D Score ≥26 (Total Score, Response, and Remission)

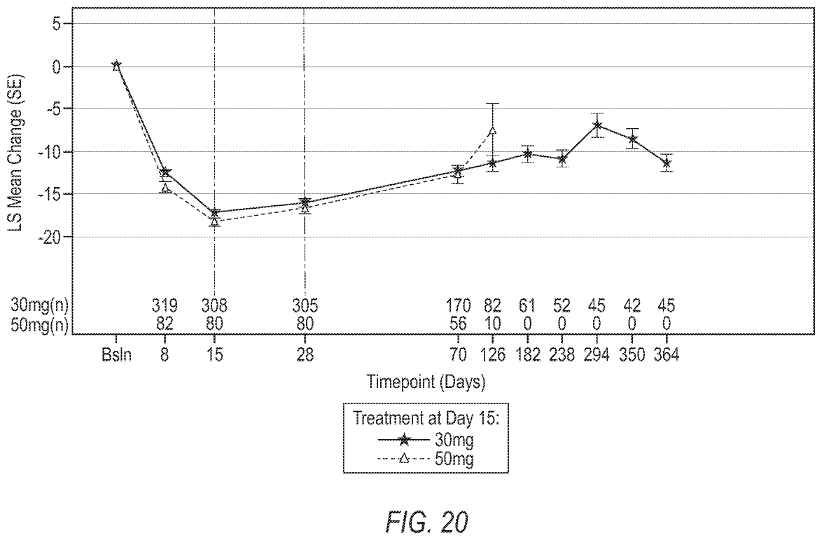

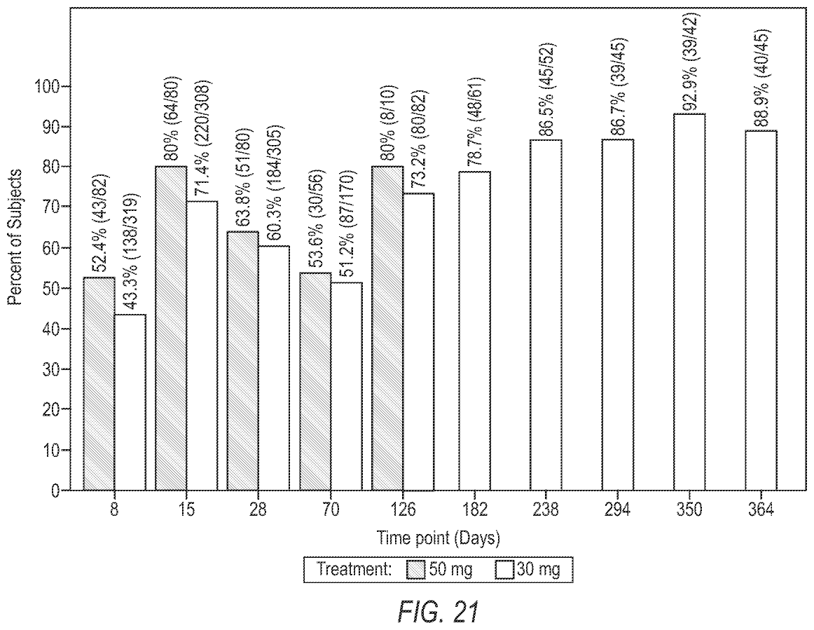

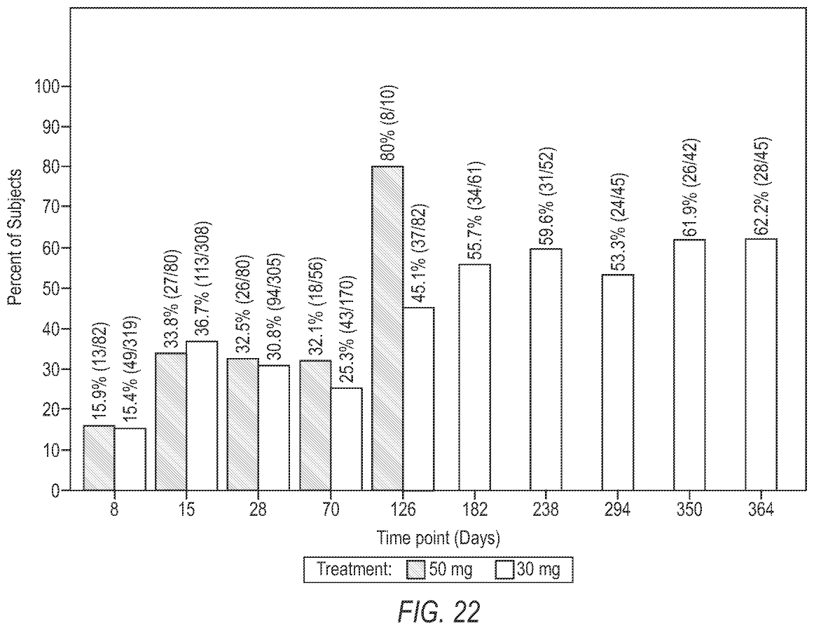

Tables 17, 18, and 19 show HAM-D total score, response, and remission, respectively at various time points of subjects who had a baseline HAM-D score ≥26. FIG. 20 provides line plots of LS mean (±SE) change from baseline for subjects who had baseline HAM-D score ≥26—Study Period 1 (safety set). FIG. 21 and FIG. 22 provide a bar chart of HAM-D Response and Remission, respectively, for Subjects Who had Baseline HAM-D Score ≥26—Study Period 1 (safety set).

TABLE 17
Mean change and percent change in HAM-D total score for subjects
who had baseline HAM-D score ≥26 - Study Period 1 (safety set)

| | 50 mg Cohort (N = 85) | | | 30 mg Cohort (N = 327) | | |
|---|---|---|---|---|---|---|
| | | Mean CFB ± | Mean % CFB ± | | Mean CFB ± | Mean % CFB ± |
| Visit | N | SD [1] | SD | N | SD [1] | SD |
| Baseline | 85 | 28.3 ± 1.99 | | 327 | 29.0 ± 2.73 | |
| Day 8 | 82 | −14.0 ± 6.05 | −49.6 ± 21.15 | 319 | −12.6 ± 7.44 | −43.7 ± 25.84 |
| Day 15 | 80 | −18.2 ± 5.89 | −64.3 ± 20.17 | 308 | −17.6 ± 7.77 | −60.6 ± 26.28 |
| Day 28 | 80 | −16.6 ± 7.05 | −58.5 ± 24.24 | 305 | −16.2 ± 8.48 | −55.6 ± 28.46 |
| Day 70 | 56 | −15.1 ± 8.23 | −52.9 ± 28.48 | 170 | −14.7 ± 9.65 | −49.8 ± 32.15 |
| Day 126 | 10 | −21.0 ± 10.12 | −74.2 ± 34.34 | 82 | −19.1 ± 8.11 | −65.6 ± 28.15 |

[1] Baseline row represents HAM-D total score mean ± SD

TABLE 18
HAM-D Response for Subjects Who had Baseline HAM-D
Score ≥26 for Study period 1 only (safety set).

| | 50 mg Cohort | 30 mg Cohort |
|---|---|---|
| Visit | (N = 85) | (N = 327) |
| Day 8 | 43/82 (52.4%) | 138/319 (43.3%) |
| Day 15 | 64/80 (80.0%) | 220/308 (71.4%) |
| Day 28 | 51/80 (63.8%) | 184/305 (60.3%) |
| Day 70 | 30/56 (53.6%) | 87/170 (51.2%) |
| Day 126 | 8/10 (80%) | 60/82 (73.2%) |

TABLE 19
HAM-D Remission for Subjects Who had Baseline HAM-D
Score ≥26 for Study period 1 only (safety set).

| | 50 mg Cohort | Low Dose Cohort |
|---|---|---|
| Visit | (N = 85) | (N = 327) |
| Day 8 | 13/82 (15.9%) | 49/319 (15.4%) |
| Day 15 | 27/80 (33.8%) | 113/308 (36.7%) |
| Day 28 | 26/80 (32.5%) | 94/305 (30.8%) |
| Day 70 | 18/56 (32.1%) | 43/170 (25.3%) |
| Day 126 | 8/10 (80%) | 37/82 (45.1%) |

### 6 Re-Treatments

The average (range) number of re-treatments per subject is 0.8 (0-4) for Low Dose Cohort, and 2.8 (1-4) for Dose Switch Cohort. Altogether, in the 30 mg Cohort, average number of re-treatments was 1.2 (0-4). At the time of this reporting, no subjects in the 50 mg Cohort had been in the study for longer than 6 months, with majority (n=114) having been followed for less than 3 months, and only 1 subject had received more than one re-treatment.

TABLE 20
Re-treatments since Treatment Cycle 1

| | 50 mg Cohort | 30 mg Cohort | Low Dose | Dose Switch |
|---|---|---|---|---|
| | (N = 199) | (N = 725) | (N = 645) | (N = 80) |
| Number of Subjects completing C1 as | 146 | 489 | 409 | 80 |
| responder (FAS) | | | | |
| Eligible to be retreated in at least one more | 122 | 472 | 392 | 80 |
| cycle [1] | | | | |
| Qualified to be retreated in at least 1 more | 36 (29.5%) | 305 (64.6%) | 225 (57.4%) | 80 (100%) |
| cycle [2, 3] | | | | |
| Dosed in at least 1 re-treatment cycle [3] | 26 (21.3%) | 279 (59.1%) | 199 (0.8%) | 80 (100%) |

[1] Eligible for re-treatment means that subject completed Treatment Cycle 1 and did not discontinue the study within 56 days after the last dose date.
[2] Qualified for re-treatment means that subject was eligible for re-treatment and had a HAM-D total score >= 20 before re-treatment.
[3] Percentages use the denominator of number of subjects from Eligible for Re-treatment.

TABLE 21
Population by Dosing Cycle (safety set)

| | 30 mg Cohort | | | |
|---|---|---|---|---|
| | Low Dose | Dose Switch | | 50 mg |
| | Cohort | Cohort | Overall | Cohort |
| Number of Subjects Dosed in C1 | 645 | 80 | 725 | 199 |
| Number (%) of Responders at D15 in C1 | 409 (63.4%) | 80 (100%) | 489 (67.4%) | 146 (73.4%) |
| who did not discontinue in C1 (FAS) [1] | | | | |
| Number (%) of FAS Subjects Dosed in C2 | 199 (48.7%) | 80 (100%) | 279 (57.1%) | 26 (17.8%) |
| Subject dosing in exactly 1 re-treatment [2] | 116 (28.4%) | 9 (11.3%) | 125 (25.6%) | 25 (17.1%) |
| Number (%) of FAS Subjects dosed in C3 [2] | 83 (20.3%) | 71 (88.8%) | 154 (31.5%) | 1 (0.7%) |
| Subject dosing in exactly 2 re-treatment [2] | 40 (9.7%) | 18 (22.5%) | 58 (11.9%) | 1 (0.7%) |
| Number (%) of FAS Subjects dosed in C4 [2] | 43 (10.5%) | 53 (66.3%) | 96 (19.6%) | NA |
| Subject dosing in exactly 3 re-treatment [2] | 23 (5.6%) | 30 (37.5%) | 53 (10.8%) | NA |
| Number (%) of FAS Subjects dosed in C5 [2] | 20 (4.9%) | 23 (28.8%) | 43 (8.8%) | NA |
| Subject dosing in exactly 4 re-treatments | 20 (4.9%) | 23 (28.8%) | 43 8.8%) | NA |

[1] Percentages based on number of subjects dosed in C1
[2] Percentages based on number of subjects in FAS

TABLE 22
Number (%) of Subjects by Treatment Cycles, by Antidepressant
Use at Study Period 1 Baseline (FAS)

| | ATD Use at Baseline = Yes | | ATD Use at Baseline = No | |
|---|---|---|---|---|
| | 50 mg Cohort | 30 mg Cohort | 50 mg Cohort | 30 mg Cohort |
| | (N = 60) | (N = 209) | (N = 86) | (N = 280) |
| No re-treatment (Cycle 1) | 48 (80.0%) | 96 (45.9%) | 72 (83.7%) | 114 (40.7%) |
| Only 1 re-treatment (Cycle 2) | 12 (20.0%) | 45 (21.5%) | 13 (15.1%) | 80 (28.6%) |
| Only 2 re-treatment (Cycle 3) | 0 | 27 (12.9%) | 1 (1.2%) | 31 (11.1%) |
| Only 3 re-treatment (Cycle 4) | NA | 20 (9.6%) | 0 | 33 (11.8%) |
| Only 4 re-treatment (Cycle 5) | NA | 21 (10.0%) | 0 | 22 (7.9%) |

TABLE 23
Summary of Time to Each Re-treatment (Days) since Last Dose
in the Previous Treatment Cycle, by Treatment Cycle (FAS)

| Treatment | | 50 mg Cohort | Low Dose | Dose Switch |
|---|---|---|---|---|
| Cycles | | (N = 146) | (N = 409) | (N = 80) |
| Cycle 2 | n/N [1] | 26/122 | 199/392 | 80/80 |
| | Mean (SD), Median, | 70.5 (18.52), 62.5, | 99.5 (52.15), 78, | 116.5 (70.26), 91, |
| | Min - Max | 58-122 | 58-297 | 58-318 |
| Cycle 3 | n/N [1] | 1/4 | 83/166 | 71/76 |
| | Mean (SD), | 58.0 (NA), 58.0, | 87.2 (34.97), 72, | 92.5 (34.44), 79, |
| | Min - Max | 58-58 | 52-217 | 58-214 |
| Cycle 4 | n/N [1] | NA | 43/74 | 53/63 |
| | Mean (SD), | | 72.1 (17.67), 65, | 69.9 (13.40), 66, |
| | Min - Max | | 57-113 | 57-120 |
| Cycle 5 | n/N [1] | NA | 20/34 | 23/45 |
| | Mean (SD), | | 63.2 (7.42) 60.5, | 61.4 (6.24), 58, |
| | Min - Max | | 58-86 | 57-79 |

[1] N = Number of FAS subjects eligible for dosing in Cycle X; n = number of FAS subjects who were eligible and got dosed in Cycle X.

TABLE 24
Summary of Time to Each Re-treatment (Days)
by Antidepressant Use at Baseline in the Previous
Treatment Cycle - Low Dose Cohort (FAS)

| | ATD Use at Baseline of Cycle X − 1= | | | | | |
|---|---|---|---|---|---|---|
| | Yes | | | No | | |
| Dosed in Cycle X, N= | N | Mean (SD) | | N | Mean (SD) | |
| Cycle 2, N = 199 | 75 | 103.3 | (56.26) | 124 | 97.2 | (49.59) |
| Cycle 3, N = 83 | 38 | 85.9 | (32.14) | 45 | 88.4 | (37.52) |
| Cycle 4, N = 43 | 20 | 71.1 | (17.21) | 23 | 73.0 | (18.40) |
| Cycle 5, N = 20 | 10 | 64.5 | (10.09) | 10 | 61.9 | (3.28) |

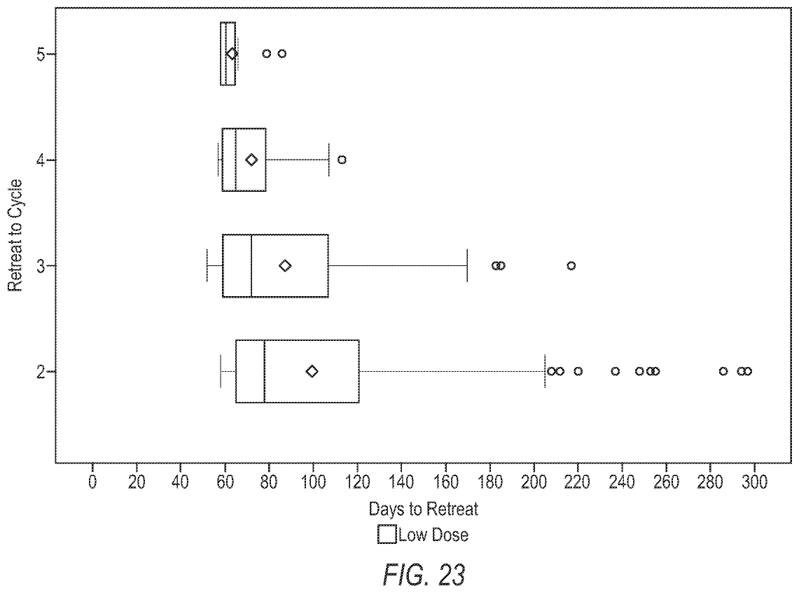

FIG. 23 provides box plots of Time to Each Re-treatment by Treatment Cycles.

### 7 Efficacy in Responders Vs Non-Responders

TABLE 25
Summary of Non-responder Status at D 15 in Past Treatment Cycles for
Responders at D 15 in Current Treatment Cycle - Low Dose Cohort (FAS)

| | | Response | Status in C2 | | | | Status in C3 | | | Status in C4 | |
|---|---|---|---|---|---|---|---|---|---|---|---|
| Cycle | # Sub. | Status at | | | Non- | | | | Non- | | Non- |
| No. | Dosed | D 15+ | Responder* | | responder* | | Responder* | | responder* | Responder* | responder* |
| C2 | 199 | Responder | NA | | NA | | NA | | NA | NA | NA |
| | | (N = 118, 59.3%) | | | | | | | | | |
| | | Non-responder | NA | | NA | | NA | | NA | NA | NA |
| | | (N = 70, 35.2%) | | | | | | | | | |
| C3 | 83 | Responder | 36 | (75.0%) | 12 | (25.0%) | NA | | NA | NA | NA |
| | | (N = 48, 57.8%) | | | | | | | | | |
| | | Non-responder | 14 | (42.4%) | 19 | (57.6%) | NA | | NA | NA | NA |
| | | (N = 33, 39.8%) | | | | | | | | | |
| C4 | 43 | Responder | 14 | (63.6%) | 8 | (36.4%) | 18 | (81.8%) | 4 (18.2%) | NA | NA |
| | | (N = 22, 51.2%) | | | | | | | | | |
| | | Non-responder | 8 | (44.4%) | 10 | (55.6%) | 9 | (50.0%) | 9 (50.0%) | NA | NA |
| | | (N = 18, 41.9%) | | | | | | | | | |
| C5 | 20 | Responder | 7 | (70.0%) | 3 | (30.0%) | 8 | (80.0%) | 2 (20.0%) | 9 (90.0%) | 1 (10.0%) |
| | | (N = 10, 50%) | | | | | | | | | |
| | | Non-responder | 1 | (14.3%) | 6 | (85.7%) | 4 | (57.1%) | 3 (42.9%) | 1 (14.4%) | 5 (71.4%) |
| | | (N = 7, 35%) | | | | | | | | | |

+Denominator for percentage is numbers of subjects dosed subjects in current cycle
*Denominator for percentage is number of responders or non-responders at current cycle.

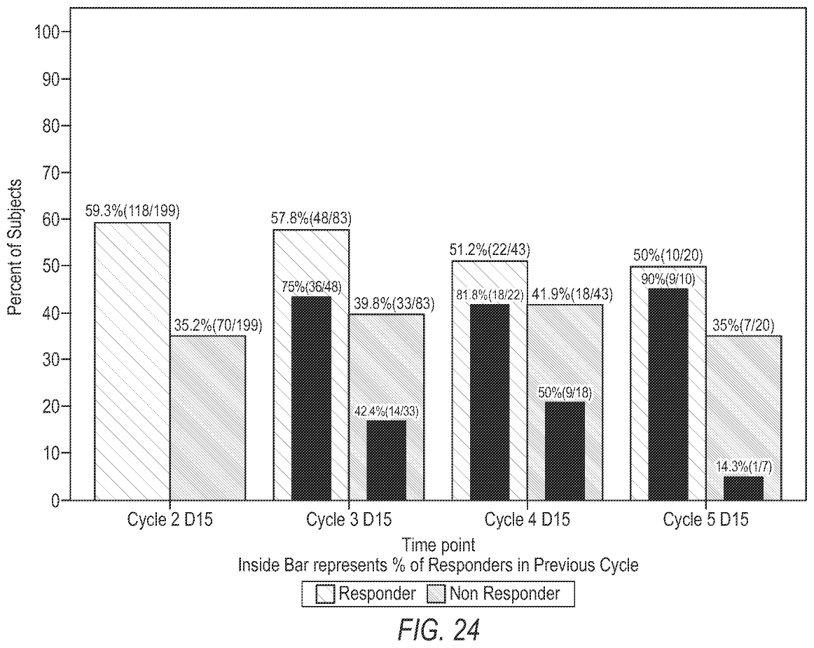

FIG. 24 provides a bar chart of HAM-D Response/Non-responders with percent of Responders in Previous Treatment Cycle-Low Dose (FAS) [Inside bars represent percent of responders in previous cycle].

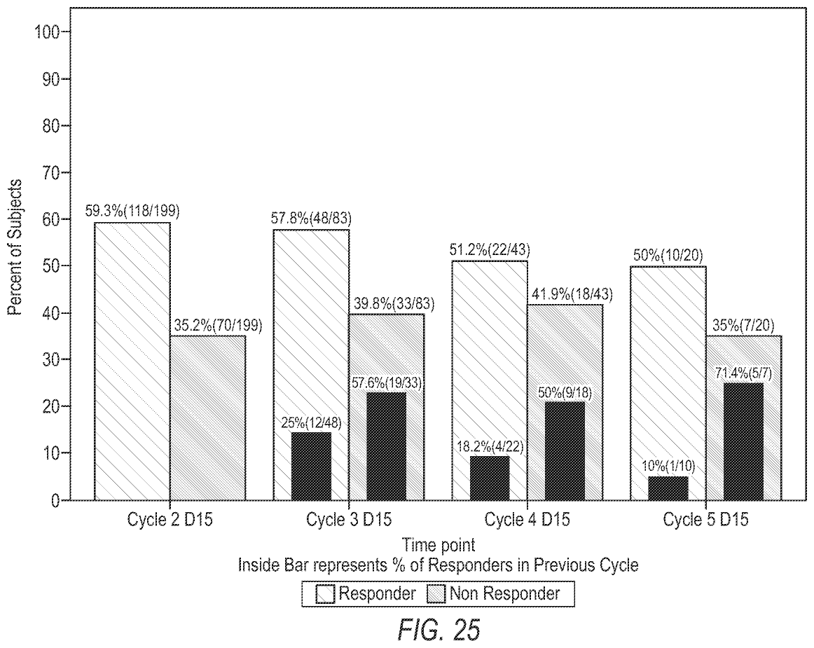

FIG. 25 provides a bar chart of HAM-D Response/Non-responders with percent of Non-responders in Previous Treatment Cycle-Low Dose (FAS) [Inside bars represent percent of non-responders in previous cycle].

### 8 HAM-D Total Score in Current and Past Cycles

### Low Dose Cohort

TABLE 26
Mean Change/Percent Change in HAM-D Total Score from Period-Specific Baseline in Current and
Past Treatment Cycles among Subjects Dosed in a Treatment Cycle - Low Dose Cohort (FAS).

| Number of | | | | | | |
|---|---|---|---|---|---|---|
| responders in | | | | | | |
| C1 dosed in | Study | n*, Mean Change/Percent Change (n) | | | | |
| Cycle X | Day | Cycle 5 | Cycle 4 | Cycle 3 | Cycle 2 | Cycle 1 |
| Cycle 2: 199 | Day 15 | NA | NA | NA | 188, −12.3/−52.6 | 199, −17.4/−70.0 |
| | Day 28 | NA | NA | NA | 188, −11.5/−49.1 | 199, −13.8/−55.2 |
| Cycle 3: 83 | Day 15 | NA | NA | 81, −12.5/−52.4 | 83, −12.7/−52.9 | 83, −17.2/−68.8 |
| | Day 28 | NA | NA | 81, −11.9/−49.8 | 83, −11.2/−46.5 | 83, −12.7, — |
| Cycle 4: 43 | Day 15 | NA | 40, −11.7/−50.0 | 43, −12.7/−54.1 | 43, −12.1/−49.6 | 43, −17.0/−68.2 |
| | Day 28 | NA | 42, −9.9/−41.9 | 43, −11.1/−46.9 | 43, −10.4/−42.4 | 43, −13.0/−52.0 |
| Cycle 5: 20 | Day 15 | 17, −11.6/— | 18, −11.6/−48.9 | 20, −11.9/−51.5 | 20, −11.1/−45.1 | 20, −16.5/−66.9 |
| | Day 28 | 16, −7.1/−30.6 | 20, −7.7/−32.6 | 2, −10.7/−46.1 | 20, −9.2/−37.8 | 20, −11.3/−45.7 |

*n = number of subjects on which the calculation is based.

### Dose Switch Cohort

TABLE 27
HAM-D Total Score - Dose Switch Cohort (FAS)

| | First | | | | | | | | |
|---|---|---|---|---|---|---|---|---|---|
| | Dose in | Study | | | | | | | |
| Endpoint | Cycle X | Day | Cycle 2 | | Cycle 3 | Cycle 4 | | Cycle 5 | |
| Number of Subjects | NA | NA | 10 | | 21 | 38 | | 11 | |
| switching to 50 mg in Cycle | | | | | | | | | |
| n*, Mean Change/Percent | 30 mg | Day | 69, −15.5/−59.1 | | 48, −15.8/−60.0 | 11, −14.8/−60.0 | | NA | |
| Change from Period- | | 15 | | | | | | | |
| Specific Baseline | | | | | | | | | |
| | | Day | 69, −12.8/— | | 49, −12.2/— | 11, −7.1/−28.3 | | NA | |
| | 50 mg | Day | 9, −14.6/−65.5 | | 22, −13.5/— | 40, −16.4/— | | 22, −17.5/— | |
| | | Day | 8, −14.8/−65.3 | | 22, −12.9/— | 39, −11.9/— | | 21, −11.2/— | |
| | 40 mg | Day | NA | | 0 | 2, −10.0/−44.1 | | 0 | |
| | | Day | NA | | 0 | 2, −14.5/−65.3 | | 0 | |
| HAM-D response | 30 mg | Day | 46/69 | (66.7%) | 34/48 | 7/11 | (63.6%) | NA | |
| | | Day | 36/69 | (52.2%) | 24/49 (49%) | 2/11 | (18.2%) | NA | |
| | 50 mg | Day | 7/9 | (77.8%) | 14/22 | 32/40 | (80%) | 18/22 | (81.8%) |

| | | | | | | |
|---|---|---|---|---|---|---|
| Day | 7/8 | (87.5%) | 14/22 | 24/39 | 11/21 | (52.4%) |

| | 40 mg | Day | NA | | 0 | 1/2 | (50%) | 0 |
|---|---|---|---|---|---|---|---|---|
| | | Day | NA | | 0 | 1/2 | (50%) | 0 |
| HAM-D remission | 30 mg | Day | 24/69 | (34.8%) | 19/48 | 3/11 | (27.3%) | NA |
| | | Day | 13/69 | (18.8%) | 5/49 (10.2%) | 1/11 | (9.1%) | NA |

| | | | | | | | |
|---|---|---|---|---|---|---|---|
| 50 mg | Day | 5/9 | (55.6%) | 4/22 (18.2%) | 19/40 | 14/22 | (63.6%) |

| | | | | | | | |
|---|---|---|---|---|---|---|---|
| Day | 3/8 | (37.5%) | 5/22 (22.7%) | 9/39 | (23.1%) | 5/21 | (23.8%) |

| | | | | | | |
|---|---|---|---|---|---|---|
| 40 mg | Day | NA | 0 | 1/2 | (50%) | 0 |
| | Day | NA | 0 | 1/2 | (50%) | 0 |
| | [symbol] | | | | | |

*n = number of subjects on which the calculation is based.
Dose Switch cohort includes subjects who started the study with 30 mg dose but switched to 50 mg in a subsequent treatment cycle. [symbol]
indicates data missing or illegible when filed

### Safety Results (Adverse Events)

Similar proportion of subjects reported treatment-emergent adverse events (TEAEs) across three dose cohorts: 60.5% ( 46/76) in 50 mg cohort and 66.6% ( 451/677) in Low Dose cohort and 77.1% ( 37/48) in Dose Switch cohort subjects.

TABLE 28
Overall Summary of Adverse Events (Safety Set)

| | | 30 mg Cohort | | | |
|---|---|---|---|---|---|
| | 50 mg Cohort | Low Dose | Dose Switch | 30 mg Cohort | Overall |
| | (N = 199) | (N = 645) | (N = 80) | (N = 725) | (N = 924) |
| Number (%) of | 125 (62.8%) | 436 (67.6%) | 57 (71.3%) | 493 (68.0%) | 618 (66.9%) |
| Subjects with at least | | | | | |
| one TEAE | | | | | |
| Number (%) of | 15 (7.5%) | 37 (5.7%) | 8 (10.0%) | 45 (6.2%) | 60 (6.5%) |
| Subjects with at | | | | | |
| least one severe | | | | | |
| TEAE | | | | | |
| Number (%) of | 13 (6.5%) | 18 (2.8%) | 1 (1.3%) | 19 (2.6%) | 32 (3.5%) |
| Subjects with at | | | | | |
| least one TEAE | | | | | |
| leading to study | | | | | |
| drug | | | | | |
| discontinuation | | | | | |
| Number (%) of | 14 (7.0%) | 31 (4.8%) | 1 (1.3%) | 32 (4.4%) | 46 (5.0%) |
| Subjects with at | | | | | |
| least one TEAE | | | | | |
| leading to study | | | | | |
| withdrawal | | | | | |
| Number (%) of | 7 (3.5%) | 19 (2.9%) | 1 (1.3%) | 20 (2.8%) | 27 (2.9%) |
| Subjects with at | | | | | |
| least one serious | | | | | |
| TEAE | | | | | |
| Study Period 1 only - Number (%) of Subjects in: | | | | | |
| Treatment Period | 108 (54.3%) | 320 (49.6%) | 28 (35.0%) | 348 (48.0%) | 456 (49.4%) |
| TEAE | | | | | |
| Treatment Cycle | 117 (58.8%) | 337 (52.2%) | 31 (38.8%) | 368 (50.8%) | 485 (52.5%) |
| Period TEAE | | | | | |

Treatment period TEAEs are those that start between first dose and last dose of Compound (1) + 1 day.
Treatment Cycle period TEAEs are those that start between first dose and 14 days after the last dose of Compound (1).

TEAEs that happened in more than 5% of subjects in any dose cohort are provided in Table 29.

TABLE 29
TEAEs that happened in more than 5% of subjects
in any dose cohort to date (safety set)

| | 50 mg | Low Dose | Dose Switch |
|---|---|---|---|
| | Cohort | Cohort | Cohort |
| | (N = 199) | (N = 645) | (N = 80) |
| Headache | 20 (10.1%) | 90 (14.0%) | 13 (16.3%) |
| Somnolence | 29 (14.6%) | 76 (11.8%) | 10 (12.5%) |
| Dizziness | 30 (15.1%) | 47 (7.3%) | 7 (8.8%) |
| Sedation | 18 (9.0%) | 29 (4.5%) | 11 (13.8%) |
| Diarrhea | 4 (2.0%) | 43 (6.7%) | 11 (13.8%) |
| Dry Mouth | 7 (3.5%) | 40 (6.2%) | 3 (3.8%) |
| Nausea | 11 (5.5%) | 25 (3.9%) | 3 (3.8%) |
| Upper Respiratory | 1 (0.5%) | 52 (8.1%) | 5 (6.3%) |
| Tract Infection | | | |
| Insomnia | 10 (5.0%) | 31 (4.8%) | 5 (6.3%) |
| Back Pain | 0 | 18 (2.8%) | 5 (6.3%) |
| Arthralgia | 1 (0.5%) | 14 (2.2%) | 5 (6.3%) |
| Fatigue | 5 (2.5%) | 27 (4.2%) | 8 (10.0%) |

The majority of the TEAEs were mild to moderate in all dose cohorts, with similar incidences across all dose cohorts.

TABLE 30
Overview of intensity of TEAEs

| | | 30 mg cohort | | |
|---|---|---|---|---|
| | 50 mg | Low Dose | Dose Switch | 30 mg |
| | Cohort | Cohort | Cohort | Cohort |
| | (N = 199) | (N = 645) | (N = 80) | (N = 725) |
| Mild | 45 (22.6%) | 187 (29.0%) | 13 (16.3%) | 200 (27.6%) |
| Moderate | 65 (32.7%) | 212 (32.9%) | 36 (45.0%) | 248 (34.2%) |
| Severe | 15 (7.5%) | 37 (5.7%) | 8 (10.0%) | 45 (6.2%) |

The overall safety observations in subjects receiving Compound (1) 50 mg is comparable with the safety findings observed with 30 mg and does not deviate from the known safety profile of Compound (1). Similar adverse events were reported regardless of the presence or absence of pre-existing antidepressant therapy. No events of loss of consciousness were reported as of the cutoff date.

During treatment cycle 1, 2.5% ( 5/199) subjects reported SAEs in the 50 mg cohort and 0.9% ( 6/645) in the Low Dose cohort. In the 50 mg cohort, three of the five SAEs were considered probably related to the study drug; drug was withdrawn, and the subject was discontinued from the study in each case: One subject with confusional state, one subject with asthenia, and one subject with delirium. In the 50 mg cohort, 2 of the 5 SAEs that were considered not related to the study drug were suicide attempt and intentional self-injury; both were resolved within 7 days.

In the Low dose cohort (30 mg only), none of the SAEs was considered probably or possibly related to the study drug. Although 5 out of 6 SAEs were considered severe in intensity, only one (Intracranial aneurysm) resulted in discontinuation from the study.

TABLE 31
Possibly or probably related SAEs

| | | Dose |
|---|---|---|
| 50 mg Cohort | Low Dose Cohort | switch |
| Confusional state | Suicidal ideation | N/A |
| (Day 6, resolved on | (Day 15, resolved on | |
| Day 8 of period 1) | Day 20 of Period 3) | |
| Asthenia | | |
| (Day 8, resolved on | | |
| Day 9 of period 1) | | |
| Delirium | | |
| (Day 9, resolved on | | |
| Day 11 of period1) | | |

The percent of subjects reporting TEAEs leading to withdrawal from study was 7.0% ( 14/199) in the 50 mg cohort, 4.8% ( 31/645) in the Low dose cohort and 1.3% ( 1/80) in the Dose switch cohort.

The percent of subjects reporting TEAEs leading to dose reduction was, in the 50 mg cohort: 17.1% ( 34/199), all but 2 in Cycle 1, 2/26 in Cycle 2. In the Low dose cohort: overall 5.0% ( 32/645): 3.4% ( 22/645) in Cycle 1, 4.4% ( 9/206) in Cycle 2, 2.3% ( 2/86) in Cycle 3, 0 ( 0/43) in Cycle 4, and 10% ( 2/20) Cycle 5, respectively. In the Dose switch cohort: overall 15.0% ( 12/80): 2.5% ( 3/80) each in Cycle 1 and Cycle 2 while on 30 mg; 4.2% ( 3/71) in Cycle 3, 3.8% ( 2/53) in Cycle 4, 17.4% ( 4/23) in Cycle 5 while switched to 50 mg. Somnolence, Dizziness, Sedation and Headache are the most common AEs that led to dose reduction.

TABLE 32
Adverse Events Overview by Antidepressant Use at Period-specific
over Study Periods - Low Dose Cohort (Safety Set)

| | ATD | Study | | Study | Study | Study | Study |
|---|---|---|---|---|---|---|---|
| | Use at | Period 1 | | Period 2 | Period 3 | Period 4 | Period 5 |
| | Baseline | (N = 645) | | (N = 206) | (N = 86) | (N = 43) | (N = 20) |
| ATD Use at Period-specific | Yes | 266 | | 88 | 45 | 19 | 11 |
| Baseline, N | | | | | | | |
| | No | 379 | | 118 | 41 | 24 | 9 |
| Number (%) of subjects | Yes | 154 | (57.9%) | 48 (54.5%) | 19 (42.2%) | 7 (36.8%) | 6 (54.5%) |
| with at least one TEAE | | | | | | | |
| | No | 256 | (67.5%) | 75 (63.6%) | 21 (51.2%) | 8 (33.3%) | 3 (33.3%) |
| Number (%) of subjects | Yes | 134 | (50.4%) | 36 (40.9%) | 14 (31.1%) | 3 (15.8%) | 4 (36.4%) |
| with at least one Treatment | | | | | | | |
| cycle period TEAE | | | | | | | |
| | No | 203 | (53.6%) | 58 (49.2%) | 14 (34.1%) | 6 (25.0%) | 2 (22.2%) |
| Number (%) of subjects | Yes | 126 | (47.4%) | 29 (33.0%) | 12 (26.7%) | 3 (15.8%) | 4 (36.4%) |
| with at least one Treatment | | | | | | | |
| period AE | | | | | | | |
| | No | 194 | (51.2%) | 51 (43.2%) | 12 (29.3%) | 5 (20.8%) | 2 (22.2%) |
| Number (%) of subjects | Yes | 6 | (2.3%) | 2 (2.3%) | 0 | 0 | 0 |
| with at least one Treatment | | | | | | | |
| cycle period severe TEAE | | | | | | | |
| | No | 10 | (2.6%) | 2 (1.7%) | 2 (4.9%) | 0 | 0 |
| Number (%) of subjects | Yes | 4 | (1.5%) | 1 (1.1%) | 0 | 0 | 0 |
| with a TEAE leading to | | | | | | | |
| study drug discontinuation | | | | | | | |
| | No | 12 | (3.2%) | 1 (0.8%) | 0 | 0 | 0 |
| Number (%) of subjects with | Yes | 5 | (1.9%) | 1 (1.1%) | 0 | 0 | 0 |
| a Treatment cycle period AE | | | | | | | |
| leading to study withdrawal | | | | | | | |
| | No | 14 | (3.7%) | 3 (2.5%) | 1 (2.4%) | 0 | 0 |
| Number (%) of subjects | Yes | 1 | (0.4%) | 1 (1.1%) | 0 | 0 | 0 |
| with at least one Treatment | | | | | | | |
| cycle period serious TEAE | | | | | | | |
| | No | 5 | (1.3%) | 0 | 1 (2.4%) | 0 | 0 |

Treatment period is defined as the first dose to last dose + 1 day. Treatment cycle is defined as the first dose to end of 14-day follow up after last dose.

### CONCLUSIONS

The first course of Compound (1) 30 mg was generally well-tolerated with safety outcomes consistent with prior studies. In the 30 mg Cohort, 68.5% of subjects did not receive more than 1 re-treatment. In the Low Dose Cohort (who were only treated with 30 mg), subjects received an average of 0.8 re-treatments (or a total of 1.8 treatments). In the 30 mg Cohort, subjects received an average of 1.2 (or a total of 2.2 treatments). Approximately 70% of patients in the 30 mg Cohort who responded to the initial treatment course used at most 1 additional treatment course.

Initial response rates were 73.5% vs 80.5% in the 30 mg and 50 mg Cohorts, respectively.

The safety and tolerability profile of Compound (1) 30 mg and Compound (1) 50 mg is consistent with that seen in earlier trials. No events of loss of consciousness have been reported in either group to date. Most TEAEs in the 30 mg Cohort and the 50 mg Cohort were mild or moderate.

### Example 4. One-year Follow-up of 50 mg Cohort

### Introduction

Example 4 provides additional data from the study described in Example 1 (A Phase 3, Open-Label, 1-Year Study Of The Safety, Tolerability, And Need For Re-Treatment With Compound (1) In Adult Subjects With Major Depressive Disorder). Examples 2-3 present data through the time period ending when the 30 mg Cohort (n=725) had completed one-year follow-up. Some early data for the 50 mg Cohort, which was available at the time the 30 mg Cohort completed one-year follow-up, is provided in Example 2-3. Example 4 presents data through a later time period, ending when the 50 mg Cohort (n=199) completed one-year follow-up. Example 4 provides additional data that has now been gathered and analyzed for the 50 mg Cohort, but does not change the data, results, or conclusions that are described for the 30 mg Cohort in Examples 2-3.

The data used in Example 4 is data with a cut-off date that is the study completion date of the last subject who started the study in the 50 mg Cohort (n=199). In Examples 2-3, all subjects in 50 mg Cohort completed Treatment Cycle 1, but some had not been in the study long enough to be eligible for any re-treatment. As of the Examples 2-3 data cut-off date, only 1 subject in 50 mg Cohort had been dosed in Treatment Cycle 3. As of the Example 4 data cut-off date, all subjects in the 50 mg Cohort completed one-year follow-up.

### Results

The mean baseline HAMD score (±SD) for the 50 mg Cohort at entry into the study was 25.1±3.29 (n=199). At baseline, 81 (40.7%) patients were on pre-existing antidepressant therapy (ADT) that was continued, while 118 (59.3%) were not on ADT.

Compound (1) 50 mg was generally well-tolerated with no new safety finding or trend identified in the data available as of the cut-off date of Example 4 for 50 mg Cohort subjects followed up to one year who received one or more Treatment Cycles. Safety was assessed during Treatment Cycles and in-between Treatment Cycles and over multiple Treatment Cycles to inform tolerability over time. 137 of 199 (68.8%) 50 mg Cohort subjects reported at least one adverse event, similar to the 30 mg Cohort (68.0%). The majority of 50 mg Cohort subjects reported treatment emergent adverse events (TEAEs) with maximum severity of mild to moderate. The most common treatment emergent adverse events (TEAEs) (reported at least 5%) were somnolence (32; 16.1%), dizziness (30; 15.1%), headache (25; 12.7%), sedation (20; 10.1%), insomnia (14; 7.0%), nausea (13; 6.5%), and tremor (11; 5.5%).

The types of TEAEs reported by 50 mg Cohort subjects were similar to those reported by 30 mg Cohort subjects. The frequency of adverse drug reactions such as somnolence, dizziness, sedation, and tremor, were higher in the 50 mg Cohort: however, the severity and outcome of TEAEs was consistent with the 30 mg Cohort and the overall safety profile of Compound (1). The percent of 50 mg Cohort subjects reporting TEAEs leading to discontinuation of study drug and withdrawal from study, respectively, were 6.5% ( 13/199) and 8.0% ( 16/199).

There was no signal for increased suicidal ideation or suicidal behavior compared to baseline in any study period or dose cohort, as measured by the Columbia-Suicide Severity Rating Scale (C-SSRS). The overall adverse event profile from the 50 mg Cohort is generally consistent with the previously reported data, and the types of TEAEs reported are similar to what has been reported across the Compound (1) clinical program.

At Day 15 (Treatment Cycle 1) the HAM-D mean change from baseline for the 50 mg Cohort was −16.0±6.04 (n=185). About 149 subjects (74.9%) achieved response (at least 50% reduction in HAM-D from baseline) and 80 subjects (40.2%) achieved remission (HAM-D less than or equal to 7). Of the 149 responders, 3 subjects withdrew from the study prior to Day 28, leaving 146 subjects in the study beyond Day 28 (i.e., out of the 199 subjects initially treated with 50 mg Compound (1), 146 or about 73.4% were included in FAS). Of the 146 subjects in the study beyond Day 28, 79.5% received at most one additional Compound (1) treatment cycle. 54.8% (n=80) received 1 treatment course in total: 24.7% (n=36) received 2 treatment courses in total: 10.3% (n=15) received 3 treatment courses in total: 6.8% (n=10) received 4 treatment courses in total; and 3.4% (n=5) received 5 treatment courses in total. About 79.5% of FAS subjects in the 50 mg Cohort did not receive more than one re-treatment.

The proportion of subjects in the 50 mg Cohort who received zero or at most 1 additional Compound (1) treatment cycle was similar regardless of use of antidepressant therapy at baseline.

### Example 5. Subpopulations of Patients in Study of Example 1

### Example 5.1—MDD and Metabolic Comorbidities

Example 5.1 provides data for patients with MDD who have metabolic comorbidities of the study described in Example 1.

### INTRODUCTION

Patients with MDD who have metabolic comorbidities face additional adverse events associated with antidepressants (e.g., weight gain, metabolic abnormalities) and often respond poorly. This example presents results from a post hoc analysis of clinical study of Example 1 in patients with MDD and metabolic comorbidities.

### Methods

The clinical study of Example 1 enrolled patients with MDD, aged 18-75 years, with HAMD-17 score ≥20 and a MADRS total score ≥28 to 1 of 2 cohorts: 30 mg Cohort and 50 mg Cohort. HAMD-17 responders (≥50% reduction from baseline) at Day 15 continued in study and were assessed for retreatment eligibility.

Patients with a metabolic comorbidity were identified by the following coded terms in patient history: obesity, hyperlipidemia, hypercholesterolemia, type-2 diabetes mellitus, type-1 diabetes mellitus, glucose tolerance impaired, hypertriglyceridemia, dyslipidemia, metabolic syndrome, and/or diabetic dyslipidemia.

### Results

The study of Example 1 enrolled 924 patients (30 mg Cohort n=725; 50 mg Cohort n=199). Of these, 253/924 (27.4%) patients had metabolic comorbidities (30 mg Cohort 197/725; 27.2%: 50 mg Cohort 56/199; 28.1%). Baseline demographics vs overall population: mean (SD) age 52.2 (12.4) vs 45.0 (14.1) years; Hispanic 30.4% vs 23.8%; antidepressant use 54.2% vs 41.8%; mean (SD) kg/m2 BMI 33.4 (6.7) vs 30.0 (6.54).

Mean (SD) CFB in HAMD-17 at Day 15 of the first treatment cycle (metabolic comorbidities subpopulation vs overall population) was −14.8 (7.10) vs −15.4 (6.9); HAMD-17 response rate was 67.3% vs 75.0%; HAMD-17 remission rate was 37.1% vs 40.8%.

For the 161/253 patients in the metabolic comorbidities subpopulation who responded to and completed the first treatment cycle, 90 (55.9%) did not require additional treatment courses during their time in the study (vs 43.7% overall): the mean (range) number of total treatment courses was 1.9 (1-5) vs 1.8 (1-5) overall.

The number of patients in the metabolic comorbidities subpopulation who had ≥1 TEAE over the study duration was 167/253 (66.0%): the majority were mild/moderate ( 148/167; 88.6%). Common (≥5%) TEAEs in this subpopulation vs overall included headache (14.2% vs 13.9%), somnolence (13.8% vs 12.8%), dizziness (9.1% vs 9.1%), diarrhea (6.7% vs 6.3%), upper respiratory infection (5.9% vs 6.3%), dry mouth (5.5% vs 5.5%), and sedation (5.1% vs 8.7%). TEAEs led to discontinuation of study drug in 8/253 (3.2%) patients; to dose reduction in 19/253 (7.5%) patients; and to withdrawal from the study in 11/253 (4.3%) patients.

### CONCLUSION

Compound (1) was generally well tolerated in patients with MDD and metabolic comorbidities, showing similar safety and efficacy outcomes to those in the overall study population. These results support the further development of Compound (1) as a potential as-needed treatment for patients with MDD, including those with metabolic comorbidities.

### Example 5.2—Post-Menopausal Women with MDD

Example 5.2 provides data for patients classified as post-menopausal women suffering from MDD of the study described in Example 1.

### INTRODUCTION

Hormonal changes during early menopause can often affect the metabolism of antidepressants (ADTs). Postmenopausal/older women may not respond well to ADTs. This example presents a post hoc analysis of the study described in Example 1 in post-menopausal women with MDD.

### Methods

The study described in Example 1 enrolled patients with MDD, aged 18-75 years, with HAMD-17≥20 and a MADRS total score ≥28 to 1 of 2 cohorts: 30-mg Cohort and 50-mg Cohort. HAMD-17 responders (≥50% reduction from baseline) at Day 15 continued in study and were assessed for repeat-treatment eligibility. Post-menopausal women aged ≥45 were identified based on follicle stimulating hormone >40 at baseline (central lab data) and if medical history coded terms contained ‘menopause’.

### Results

The study described in Example 1 enrolled 924 patients (30 mg Cohort n=725; 50 mg Cohort n=199): 152/924 (16.5%) post-menopausal women received either Compound (1) 30 mg ( 110/725; 15.2%) or Compound (1) 50 mg ( 42/199; 21.1%) in the first 14-day treatment course. Other than age and sex, patient baseline demographics in this subpopulation were generally consistent with that of the overall study population, respectively: mean (SD) age, 58.7 (6.72) vs 45.0 (14.1) years; mean (SD) HAMD-17, 25.7 (3.92) vs 25.3 (3.9); antidepressant use, 52.6% vs 41.7%.

Mean (SD) CFB in HAMD-17 at Day 15 (last day of first treatment period) in post-menopausal patients was −16.0 (7.00) vs. −15.4 (6.9) in the overall study population. HAMD-17 response rate at Day 15 was 75.7% vs. 75.0% in the overall study population. HAMD-17 remission rate at Day 15 was 39.6% vs. 40.8% in the overall study population.

For the 106/152 post-menopausal patients who responded to and completed treatment cycle 1, 45 (42.5%) did not require additional courses during their time in the study; the mean (range) number of total treatment courses was 2.2 (1-5).

The number of post-menopausal patients who had at least 1 TEAE over the study duration was 102/152 (67.1%). The majority experienced TEAEs that were mild or moderate ( 90/102; 88.2%). The most common (≥5%) TEAEs in post-menopausal patients (vs overall population) included headache (13.2% vs 13.9%), somnolence (12.5% vs 12.8%), dizziness (11.8% vs 9.1%), diarrhea (7.9% vs 6.3%), upper respiratory infection (7.9% vs 6.3%), dry mouth (7.2% vs 5.5%), sedation (6.6% vs 8.7%), and insomnia (6.6% vs 5.4%). TEAEs led to discontinuation of study drug in 8/152 (5.3%) patients; to dose reduction in 16/152 (10.5%) patients; and to withdrawal from the study in 9/152 (5.9%) patients.

### CONCLUSION

Compound (1) was generally well-tolerated in post-menopausal women, showing similar safety and efficacy results to that of the overall study population. These results support the further development of Compound (1) as a potential as-needed treatment for patients with MDD, including difficult to treat post-menopausal women.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists. e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A method of treating major depressive disorder (MDD) in a subject in need thereof, comprising:
(i) performing an initial treatment course on the subject comprising administering a therapeutically effective amount of Compound (1): 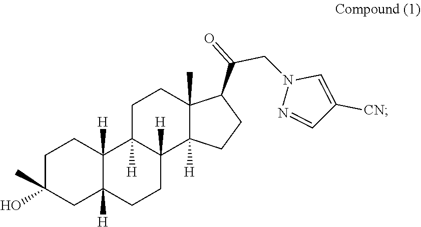
and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course.

2. A method of treating major depressive disorder (MDD) in a subject in need thereof, comprising:
(i) performing an initial treatment course on the subject comprising administering a therapeutically effective amount of a pharmaceutically acceptable salt of Compound (1): 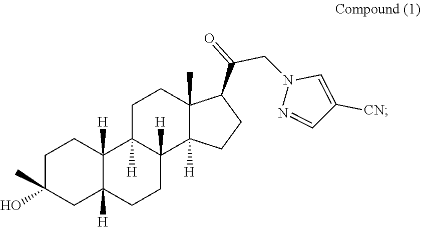
and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course.

3. The method of claim 1 or 2, wherein 0 or 1 subsequent treatment courses are performed.

4. The method of claim 1 or 2, wherein 1 subsequent treatment course is performed.

5. The method of any one of claims 1-4, wherein there is at least about a 4 week, at least about a 6 week, or at least about an 8 week interval between the end of the initial treatment course and the beginning of the subsequent treatment course.

6. The method of any one of claims 1-4, wherein there is about a 4 week, about a 6 week, or about an 8 week interval between the end of the initial treatment course and the beginning of the subsequent treatment course.

7. The method of any one of claims 1-6, wherein the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof.

8. The method of any one of claims 1-7, wherein the recurrence of depression symptoms in the subject is indicated by a PHQ-9 score greater than or equal to 10 or a HAM-D score greater than or equal to 20.

9. The method of any one of claims 1-8, wherein the initial treatment course has a duration of about 2 weeks or about 14 days.

10. The method of any one of claims 1-9, wherein each subsequent treatment course has a duration of about 2 weeks or about 14 days.

11. The method of any one of claims 1-10, wherein Compound (1), or the pharmaceutically acceptable salt of Compound (1), is administered once a day for about 14 days in the initial treatment course.

12. The method of any one of claims 1-11, wherein Compound (1), or the pharmaceutically acceptable salt of Compound (1), is administered once a day for about 14 days in each subsequent treatment course.

13. The method of claim 1, wherein Compound (1) is administered at a dose of about 20 mg to about 55 mg.

14. The method of claim 1, wherein Compound (1) is administered at a dose of about 50 mg.

15. The method of claim 1, wherein Compound (1) is administered at a dose of about 40 mg.

16. The method of claim 2, wherein the pharmaceutically acceptable salt of Compound (1) is administered at a dose equivalent to about 20 mg to about 55 mg of the free base compound.

17. The method of claim 2, wherein the pharmaceutically acceptable salt of Compound (1) is administered at a dose equivalent to about 50 mg of the free base compound.

18. The method of claim 2, wherein the pharmaceutically acceptable salt of Compound (1) is administered at a dose equivalent to about 40 mg of the free base compound.

19. The method of any one of claims 1-18, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally.

20. The method of claim 19, wherein Compound (1), or the pharmaceutically acceptable salt of Compound (1), is administered orally.

21. The method of any one of claims 1-20, wherein Compound (1), or the pharmaceutically acceptable salt of Compound (1), is administered with food.

22. The method of any one of claims 1-21, wherein Compound (1), or the pharmaceutically acceptable salt of Compound (1), is administered once a day at night.

23. The method of claim 1, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

24. The method of claim 1, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

25. The method of claim 1, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

26. The method of claim 1, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

27. The method of any one of claims 1-26, wherein the subject is treatment naïve.

28. The method of any one of claims 1-26, wherein the subject has been on a stable dose of an additional antidepressant for at least 60 days prior to the beginning of the initial treatment course.

29. A method of treating major depressive disorder (MDD) in a subject in need thereof, comprising:
(i) performing an initial treatment course on the subject comprising administering a therapeutically effective amount of Compound (1): 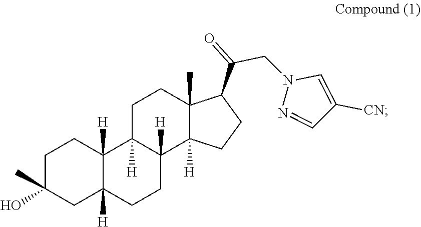
and
(ii) performing 0, 1, or 2 subsequent treatment courses on the subject, wherein each subsequent treatment course comprises administering a therapeutically effective amount of Compound (1) in response to a recurrence of depression symptoms,
wherein the 0, 1, or 2 subsequent treatment courses are performed over a period of 12 months from the beginning of the initial treatment course, and
wherein the subject is treatment naïve.

30. A method of treating major depressive disorder (MDD) in a subject in need thereof, comprising:
(i) performing an initial treatment course on the subject comprising administering a therapeutically effective amount of a pharmaceutically acceptable salt of Compound (1): 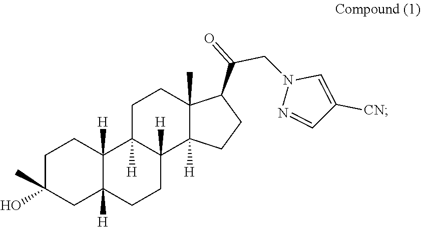
and
(ii) performing 0, 1, or 2 subsequent treatment courses on the subject, wherein each subsequent treatment course comprises administering a therapeutically effective amount of a pharmaceutically acceptable salt of Compound (1) in response to a recurrence of depression symptoms,
wherein the 0, 1, or 2 subsequent treatment courses are performed over a period of 12 months from the beginning of the initial treatment course, and
wherein the subject is treatment naïve.

31. The method of claim 29 or 30, wherein 0 or 1 subsequent treatment courses are performed.

32. The method of claim 29 or 30, wherein 1 subsequent treatment course is performed.

33. The method of any one of claims 29-32, wherein there is at least about a 4 week, at least about a 6 week, or at least about an 8 week interval between the end of the initial treatment course and the beginning of the subsequent treatment course.

34. The method of any one of claims 29-32, wherein there is about a 4 week, about a 6 week, or about an 8 week interval between the end of the initial treatment course and the beginning of the subsequent treatment course.

35. The method of any one of claims 29-34, wherein the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof.

36. The method of any one of claims 29-35, wherein the recurrence of depression symptoms in the subject is indicated by a PHQ-9 score greater than or equal to 10 or a HAM-D score greater than or equal to 20.

37. The method of any one of claims 29-36, wherein the initial treatment course has a duration of about 2 weeks or about 14 days.

38. The method of any one of claims 29-37, wherein each subsequent treatment course has a duration of about 2 weeks or about 14 days.

39. The method of any one of claims 29-38, wherein Compound (1), or the pharmaceutically acceptable salt of Compound (1), is administered once a day for about 14 days in the initial treatment course.

40. The method of any one of claims 29-39, wherein Compound (1), or the pharmaceutically acceptable salt of Compound (1), is administered once a day for about 14 days in each subsequent treatment course.

41. The method of claim 29, wherein Compound (1) is administered at a dose of about 20 mg to about 55 mg.

42. The method of claim 29, wherein Compound (1) is administered at a dose of about 50 mg.

43. The method of claim 29, wherein Compound (1) is administered at a dose of about 40 mg.

44. The method of claim 30, wherein the pharmaceutically acceptable salt of Compound (1) is administered at a dose equivalent to about 20 mg to about 55 mg of the free base compound.

45. The method of claim 30, wherein the pharmaceutically acceptable salt of Compound (1) is administered at a dose equivalent to about 50 mg of the free base compound.

46. The method of claim 30, wherein the pharmaceutically acceptable salt of Compound (1) is administered at a dose equivalent to about 40 mg of the free base compound.

47. The method of any one of claims 29-46, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally.

48. The method of claim 47, wherein Compound (1), or the pharmaceutically acceptable salt of Compound (1), is administered orally.

49. The method of any one of claims 29-48, wherein Compound (1), or the pharmaceutically acceptable salt of Compound (1), is administered with food.

50. The method of any one of claims 29-49, wherein Compound (1), or the pharmaceutically acceptable salt of Compound (1), is administered once a day at night.

51. The method of claim 29, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

52. The method of claim 29, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

53. The method of claim 29, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

54. The method of claim 29, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

55. A method of treating major depressive disorder (MDD) in a subject in need thereof, comprising:
(i) performing an initial treatment course on the subject comprising administering about 30 mg to about 50 mg of Compound (1): 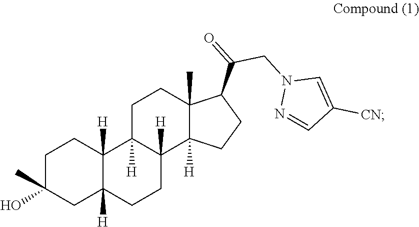
and
(ii) performing 0, 1, or 2 subsequent treatment courses on the subject, wherein each subsequent treatment course comprises administering about 30 mg to about 50 mg of Compound (1) to the subject in response to a recurrence of depression symptoms,
wherein the 0, 1, or 2 subsequent treatment courses are performed over a period of 12 months from the beginning of the initial treatment course.

56. A method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound: 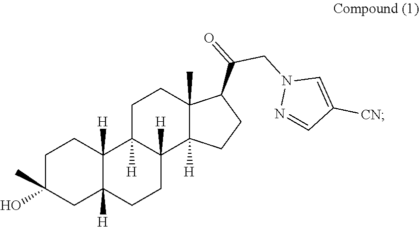
and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound to the subject in response to a recurrence of depression symptoms,
wherein the 0, 1, or 2 subsequent treatment courses are performed over a period of 12 months from the beginning of the initial treatment course.

57. A method of treating major depressive disorder (MDD) in a subject in need thereof, comprising:
(i) performing an initial treatment course on the subject comprising administering about 30 mg to about 50 mg of Compound (1) once a day for about 14 days: 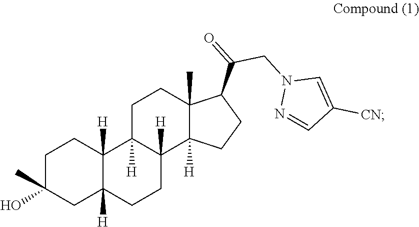
and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course.

58. A method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days: 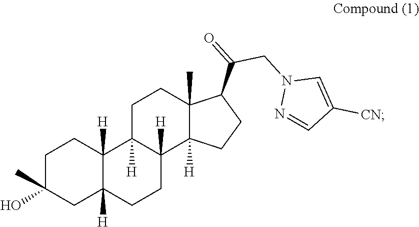
and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course.

59. A method of treating major depressive disorder (MDD) in a subject in need thereof, comprising:
(i) performing an initial treatment course on the subject comprising administering about 30 mg to about 50 mg of Compound (1) once a day for about 14 days: 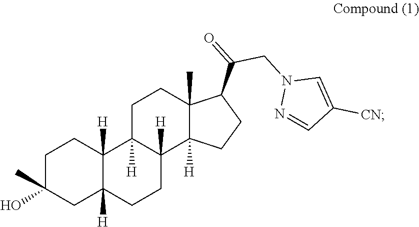
and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

60. A method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days: 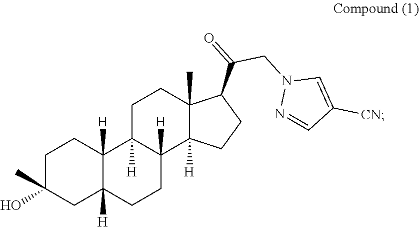
and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

61. A method of treating major depressive disorder (MDD) in a subject in need thereof, comprising:
(i) performing an initial treatment course on the subject comprising administering about 30 mg to about 50 mg of Compound (1) once a day for about 14 days: 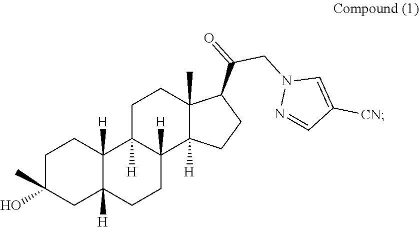
and
(ii) performing 0, 1, or 2 subsequent treatment courses on the subject, wherein each subsequent treatment course comprises administering about 30 mg to about 50 mg of Compound (1) once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

62. A method of treating major depressive disorder (MDD) in a subject in need thereof, comprising:
(i) performing an initial treatment course on the subject comprising administering a pharmaceutically acceptable salt of Compound (1) at a dose equivalent to about 30 mg to about 50 mg of the free base compound once a day for about 14 days: 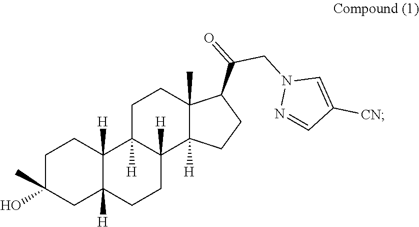
and
(ii) performing 0, 1, or 2 subsequent treatment courses on the subject, wherein each subsequent treatment course comprises administering a pharmaceutically acceptable salt of Compound (1) at a dose equivalent to about 30 mg to about 50 mg of the free base compound once a day for about 14 days to the subject in response to a recurrence of depression symptoms,
wherein the 0, 1, or 2 subsequent treatment courses are performed over a period of 12 months from the beginning of the initial treatment course, and
wherein the subject has been on a stable dose of an additional antidepressant for at least 60 days.

63. A method of treating major depressive disorder (MDD) in a subject in need thereof, comprising:
(i) performing an initial treatment course on the subject comprising administering about 45 mg to about 55 mg of Compound (1): 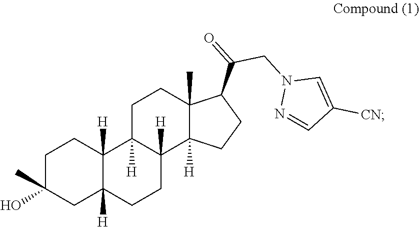
and
(ii) performing 0, 1, or 2 subsequent treatment courses on the subject, wherein each subsequent treatment course comprises administering about 45 mg to about 55 mg of Compound (1) to the subject in response to a recurrence of depression symptoms,
wherein the 0, 1, or 2 subsequent treatment courses are performed over a period of 12 months from the beginning of the initial treatment course.

64. A method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound: 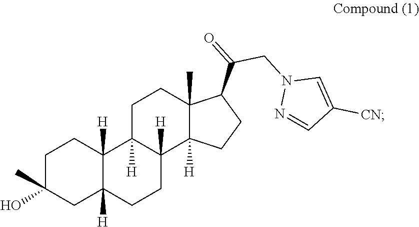
and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound to the subject in response to a recurrence of depression symptoms,
wherein the 0, 1, or 2 subsequent treatment courses are performed over a period of 12 months from the beginning of the initial treatment course.

65. A method of treating major depressive disorder (MDD) in a subject in need thereof, comprising:
(i) performing an initial treatment course on the subject comprising administering about 45 mg to about 55 mg of Compound (1) once a day for about 14 days: 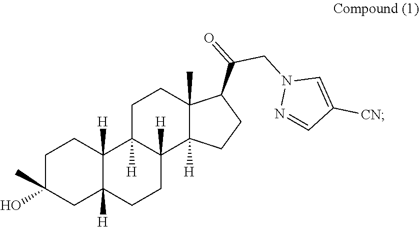
and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms,
wherein the 0, 1, or 2 subsequent treatment courses are performed over a period of 12 months from the beginning of the initial treatment course.

66. A method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days: 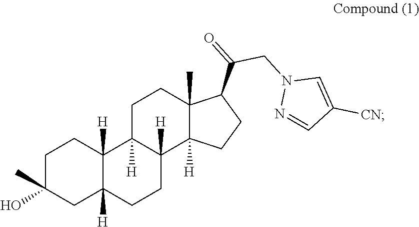
and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms,
wherein the 0, 1, or 2 subsequent treatment courses are performed over a period of 12 months from the beginning of the initial treatment course.

67. A method of treating major depressive disorder (MDD) in a subject in need thereof, comprising:
(i) performing an initial treatment course on the subject comprising administering about 45 mg to about 55 mg of Compound (1) once a day for about 14 days: 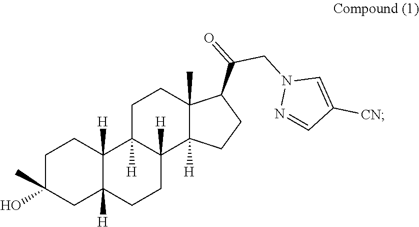
and
(ii) performing 0, 1, or 2 subsequent treatment courses on the subject, wherein each subsequent treatment course comprises administering about 45 mg to about 55 mg of Compound (1) once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

68. A method of treating major depressive disorder (MDD) in a subject in need thereof, comprising:
(i) performing an initial treatment course on the subject comprising administering a pharmaceutically acceptable salt of Compound (1) at a dose equivalent to about 45 mg to about 55 mg of the free base compound once a day for about 14 days: 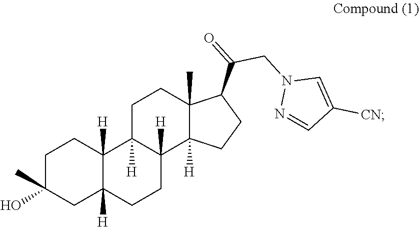
and
(ii) performing 0, 1, or 2 subsequent treatment courses on the subject, wherein each subsequent treatment course comprises administering a pharmaceutically acceptable salt of Compound (1) at a dose equivalent to about 45 mg to about 55 mg of the free base compound once a day for about 14 days to the subject in response to a recurrence of depression symptoms, provided there is at least about a 6 week interval between the end of the initial treatment course and the beginning of the subsequent treatment course,
wherein the 0, 1, or 2 subsequent treatment courses are performed over a period of 12 months from the beginning of the initial treatment course.

69. The method of any one of claims 55-68, wherein 0 or 1 subsequent treatment courses are performed.

70. The method of any one of claims 55-68, wherein the recurrence of depression symptoms is indicated by an evaluation of the subject using the Hamilton Rating Scale for Depression (HAM-D), Montgomery-Asberg Depression Rating Scale (MADRS), the Patient Health Questionnaire (PHQ-9), or a combination thereof.

71. The method of any one of claims 55-68, wherein the recurrence of depression symptoms in the subject is indicated by a PHQ-9 score greater than or equal to 10 or a HAM-D score greater than or equal to 20.

72. The method of any one of claims 55-68, wherein Compound (1) is administered at a dose of about 50 mg or the pharmaceutically acceptable salt of Compound (1) is administered at a dose equivalent to about 50 mg of the free base compound.

73. The method of any one of claims 55-68, wherein Compound (1) is administered at a dose of about 40 mg or the pharmaceutically acceptable salt of Compound (1) is administered at a dose equivalent to about 40 mg of the free base compound.

74. The method of any one of claims 55-68, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally.

75. The method of claim 74, wherein Compound (1), or the pharmaceutically acceptable salt of Compound (1), is administered orally.

76. The method of any one of claims 55-68, wherein Compound (1), or the pharmaceutically acceptable salt of Compound (1), is administered with food.

77. The method of any one of claims 55-68, wherein Compound (1), or the pharmaceutically acceptable salt of Compound (1), is administered once a day at night.

78. The method of any one of claims 55-60 and 63-68, wherein the subject is treatment naïve.
